SEQUENCE LISTING (208 sequences)

SEQ ID NO: 1 - DNA, 38 bp, Artificial Sequence
  Synthetic
cgtattggacatttccgtagaccgactggacatcttcg

SEQ ID NO: 2 - DNA, 24 bp, Artificial Sequence
  Synthetic
tggtccttcacaccaatacggcat

SEQ ID NO: 3 - DNA, 42 bp, Artificial Sequence
  Synthetic
tctacggaaatgtggcagaatcaatcataagacaccagtcgg

SEQ ID NO: 4 - DNA, 26 bp, Artificial Sequence
  Synthetic
cagacgaagatgtggtagtggaatgc

SEQ ID NO: 5 - DNA, 38 bp, Artificial Sequence
  Synthetic
tccactacctgtcttatgattgattctgcctgtgaagg

SEQ ID NO: 6 - DNA, 38 bp, Artificial Sequence
  Synthetic
ctcagtggacagccgttctggagcgttggacgaaactc

SEQ ID NO: 7 - DNA, 24 bp, Artificial Sequence
  Synthetic
tctggtagagcaccactgagaggt

SEQ ID NO: 8 - DNA, 42 bp, Artificial Sequence
  Synthetic
ccagaacggctgtggctaaacagtaaccgaagcaccaacgct

SEQ ID NO: 9 - DNA, 26 bp, Artificial Sequence
  Synthetic
accagagtttcgtggtcatcgtacct

SEQ ID NO: 10 - DNA, 38 bp, Artificial Sequence
  Synthetic
acgatgacctgcttcggttactgtttagcctgctctac

SEQ ID NO: 11 - DNA, 54 bp, Artificial Sequence
  Synthetic
acaaacaactgcctatcacgacgcttttgcgtcgtgttttcggaacctgagact

SEQ ID NO: 12 - DNA, 54 bp, Artificial Sequence
  Synthetic
tcggcattccgccgccgtcgctgcttttgcagcgacttagcattgatgatattc

SEQ ID NO: 13 - DNA, 54 bp, Artificial Sequence
  Synthetic
attgagggaatcagtacggagcacttttgtgctccgttgcgacagacgttttca

SEQ ID NO: 14 - DNA, 54 bp, Artificial Sequence
  Synthetic
gaaggaaaaatagaaagcctagcgttttcgctaggcttattcatatttcaaccg

SEQ ID NO: 15 - DNA, 54 bp, Artificial Sequence
  Synthetic
ctttacagtatcttaccgctcgtgttttcacgagcgttcgaagcccagttacca

SEQ ID NO: 16 - DNA, 54 bp, Artificial Sequence
  Synthetic
cctcaagatcccaatccgtggagcttttgctccacgttcaaataagatagcagc

SEQ ID NO: 17 - DNA, 54 bp, Artificial Sequence
  Synthetic
aatgccccataaatccgctcggacttttgtccgagctttcattaaaagaaccac

SEQ ID NO: 18 - DNA, 54 bp, Artificial Sequence
  Synthetic
caccagagttcggtcagccgagcgttttcgctcggctttagccccctcgatagc

SEQ ID NO: 19 - DNA, 54 bp, Artificial Sequence
  Synthetic
agcaccgtagggaaggtcggaggcttttgcctccgatttaaatattttattttg

SEQ ID NO: 20 - DNA, 54 bp, Artificial Sequence
  Synthetic
tcacaatcccgaggaactggtggcttttgccaccagttacgcaataatgaaata

SEQ ID NO: 21 - DNA, 54 bp, Artificial Sequence
  Synthetic
gcaatagcagagaataccgcaggcttttgcctgcggttacataaaaacagccat

SEQ ID NO: 22 - DNA, 54 bp, Artificial Sequence
  Synthetic
attatttagaaggattgccatcgcttttgcgatggcttaggattagaaacagtt

SEQ ID NO: 23 - DNA, 54 bp, Artificial Sequence
  Synthetic
ggaaagcggtaacagtgtggcagcttttgctgccacttgcccgtatcggggttt

SEQ ID NO: 24 - DNA, 54 bp, Artificial Sequence
  Synthetic
gtttgccacctcagagaccaggcgttttcgcctggtttccgccaccgccagaat

SEQ ID NO: 25 - DNA, 54 bp, Artificial Sequence
  Synthetic
ttattcatgtcaccaagctcgctgttttcagcgagctttgaaaccattattagc

SEQ ID NO: 26 - DNA, 54 bp, Artificial Sequence
  Synthetic
atacccaaacaccacgcctaccgcttttgcggtaggttgaataagtgacggaaa

SEQ ID NO: 27 - DNA, 54 bp, Artificial Sequence
  Synthetic
gcgcattaataagagcctggacgcttttgcgtccagttaagaaacaataacgga

SEQ ID NO: 28 - DNA, 54 bp, Artificial Sequence
  Synthetic
tgctcagtgccagttaggtggtcgttttcgaccaccttcaaaataaacagggaa

SEQ ID NO: 29 - DNA, 54 bp, Artificial Sequence
  Synthetic
tgccttgacagtctctgtcggtgcttttgcaccgacttgaatttacccctcaga

SEQ ID NO: 30 - DNA, 54 bp, Artificial Sequence
  Synthetic
gccaccactcttttcacggtcggcttttgccgaccgtttaatcaaatagcaagg

SEQ ID NO: 31 - DNA, 54 bp, Artificial Sequence
  Synthetic
ccggaaactaaaggtggacctggcttttgccaggtcttaattatcataaaagaa

SEQ ID NO: 32 - DNA, 54 bp, Artificial Sequence
  Synthetic
acgcaaagaagaactgtcggctcgttttcgagccgattgcatgatttgagttaa

SEQ ID NO: 33 - DNA, 54 bp, Artificial Sequence
  Synthetic
gcccaatagacgggagcacaggcgttttcgcctgtgttaattaactttccagag

SEQ ID NO: 34 - DNA, 54 bp, Artificial Sequence
  Synthetic
cctaatttaccaggcgtcggagcgttttcgctccgattgataagtgggggtcag

SEQ ID NO: 35 - DNA, 45 bp, Artificial Sequence
  Synthetic
cagccaagacgcaggtagcgagacagagctgaaagtattaagagg

SEQ ID NO: 36 - DNA, 42 bp, Artificial Sequence
  Synthetic
tcgctacctgcgttcgtcggatggtgaggtccacgctctgtc

SEQ ID NO: 37 - DNA, 45 bp, Artificial Sequence
  Synthetic
ctattattctgaaacagtggacctcaccatccgacgacacgagca

SEQ ID NO: 38 - DNA, 45 bp, Artificial Sequence
  Synthetic
cacggagtcgaagcgtaggacggtagccagtcagacgattggcct

SEQ ID NO: 39 - DNA, 42 bp, Artificial Sequence
  Synthetic
gtcctacgcttcggaccttggtgatgctggactgtggctacc

SEQ ID NO: 40 - DNA, 45 bp, Artificial Sequence
  Synthetic
caggaggttgaggcagcagtccagcatcaccaaggtcgctcggca

SEQ ID NO: 41 - DNA, 45 bp, Artificial Sequence
  Synthetic
cagccaagcggcatggtcttgcgttggaggcgtcagactgtagcg

SEQ ID NO: 42 - DNA, 42 bp, Artificial Sequence
  Synthetic
caagaccatgccgacctcatcctcgctttcggtgctccaacg

SEQ ID NO: 43 - DNA, 45 bp, Artificial Sequence
  Synthetic
atcaagtttgcctttacaccgaaagcgaggatgaggtcacgagca

SEQ ID NO: 44 - DNA, 45 bp, Artificial Sequence
  Synthetic
cacggagtctacggcagtgaccgatctccagacaaaagggcgaca

SEQ ID NO: 45 - DNA, 42 bp, Artificial Sequence
  Synthetic
gtcactgccgtagctcacgaggcacaaccacagcggagatcg

SEQ ID NO: 46 - DNA, 45 bp, Artificial Sequence
  Synthetic
ggtttaccagcgccaagctgtggttgtgcctcgtgaggctcggca

SEQ ID NO: 47 - DNA, 45 bp, Artificial Sequence
  Synthetic
cagccaaggttccacaggactcgcacttcgcagatagccgaacaa

SEQ ID NO: 48 - DNA, 42 bp, Artificial Sequence
  Synthetic
agtcctgtggaacaccacgagacgccatcgagcggaagtgcg

SEQ ID NO: 49 - DNA, 45 bp, Artificial Sequence
  Synthetic
tttttaagaaaagtaacgctcgatggcgtctcgtggtcacgagca

SEQ ID NO: 50 - DNA, 45 bp, Artificial Sequence
  Synthetic
cacggagtcaaggctacgagtcagacaggaacgtcaaaaatgaaa

SEQ ID NO: 51 - DNA, 42 bp, Artificial Sequence
  Synthetic
actcgtagccttggaccgcactcaccactgctcgcctgtctg

SEQ ID NO: 52 - DNA, 45 bp, Artificial Sequence
  Synthetic
aaacgattttttgtttcgagcagtggtgagtgcggtcgctcggca

SEQ ID NO: 53 - DNA, 31 bp, Artificial Sequence
  Synthetic
tgtatcttggttgctcgtgcttggctggcat

SEQ ID NO: 54 - DNA, 31 bp, Artificial Sequence
  Synthetic
actcgtgtctgtgccgagcactccgtgaggt

SEQ ID NO: 55 - DNA, 31 bp, Artificial Sequence
  Synthetic
atgccagccaagcacgagcaaccaagataca

SEQ ID NO: 56 - DNA, 31 bp, Artificial Sequence
  Synthetic
acctcacggagtgctcggcacagacacgagt

SEQ ID NO: 57 - DNA, 45 bp, Artificial Sequence
  Synthetic
cagagccaacgcaggtagcgagacagagctgaaagtattaagagg

SEQ ID NO: 58 - DNA, 42 bp, Artificial Sequence; identical to SEQ ID NO: 36
  Synthetic

SEQ ID NO: 59 - DNA, 45 bp, Artificial Sequence
  Synthetic
ctattattctgaaacagtggacctcaccatccgacgagcggacga

SEQ ID NO: 60 - DNA, 45 bp, Artificial Sequence
  Synthetic
tgcgaaggcgaagcgtaggacggtagccagtcagacgattggcct

SEQ ID NO: 61 - DNA, 42 bp, Artificial Sequence; identical to SEQ ID NO: 39
  Synthetic

SEQ ID NO: 62 - DNA, 45 bp, Artificial Sequence
  Synthetic
caggaggttgaggcagcagtccagcatcaccaaggtctcgcagga

SEQ ID NO: 63 - DNA, 45 bp, Artificial Sequence
  Synthetic
cagagccacggcatggtcttgcgttggaggcgtcagactgtagcg

SEQ ID NO: 64 - DNA, 42 bp, Artificial Sequence; identical to SEQ ID NO: 42
  Synthetic

SEQ ID NO: 65 - DNA, 45 bp, Artificial Sequence
  Synthetic
atcaagtttgcctttacaccgaaagcgaggatgaggtgcggacga

SEQ ID NO: 66 - DNA, 45 bp, Artificial Sequence
  Synthetic
tgcgaaggctacggcagtgaccgatctccagacaaaagggcgaca

SEQ ID NO: 67 - DNA, 42 bp, Artificial Sequence; identical to SEQ ID NO: 45
  Synthetic

SEQ ID NO: 68 - DNA, 45 bp, Artificial Sequence
  Synthetic
ggtttaccagcgccaagctgtggttgtgcctcgtgagtcgcagga

SEQ ID NO: 69 - DNA, 45 bp, Artificial Sequence
  Synthetic
cagagccagttccacaggactcgcacttcgcagatagccgaacaa

SEQ ID NO: 70 - DNA, 42 bp, Artificial Sequence; identical to SEQ ID NO: 48
  Synthetic

SEQ ID NO: 71 - DNA, 45 bp, Artificial Sequence
  Synthetic
tttttaagaaaagtaacgctcgatggcgtctcgtggtgcggacga

SEQ ID NO: 72 - DNA, 45 bp, Artificial Sequence
  Synthetic
tgcgaaggcaaggctacgagtcagacaggaacgtcaaaaatgaaa

SEQ ID NO: 73 - DNA, 42 bp, Artificial Sequence; identical to SEQ ID NO: 51
  Synthetic

SEQ ID NO: 74 - DNA, 45 bp, Artificial Sequence
  Synthetic
aaacgattttttgtttcgagcagtggtgagtgcggtctcgcagga

SEQ ID NO: 75 - DNA, 31 bp, Artificial Sequence
  Synthetic
cgttatctggttcgtccgctggctctggcat

SEQ ID NO: 76 - DNA, 31 bp, Artificial Sequence
  Synthetic
cgttatctctgtcctgcgaccttcgcaaggt

SEQ ID NO: 77 - DNA, 31 bp, Artificial Sequence
  Synthetic
atgccagagccagcggacgaaccagataacg

SEQ ID NO: 78 - DNA, 31 bp, Artificial Sequence
  Synthetic
accttgcgaaggtcgcaggacagagataacg

SEQ ID NO: 79 - DNA, 45 bp, Artificial Sequence
  Synthetic
aagataagacgcaggtagcgagacagagctgaaagtattaagagg

SEQ ID NO: 80 - DNA, 42 bp, Artificial Sequence; identical to SEQ ID NO: 36
  Synthetic

SEQ ID NO: 81 - DNA, 45 bp, Artificial Sequence
  Synthetic
ctattattctgaaacagtggacctcaccatccgacgaatacacgg

SEQ ID NO: 82 - DNA, 45 bp, Artificial Sequence
  Synthetic
acctaaagcgaagcgtaggacggtagccagtcagacgattggcct

SEQ ID NO: 83 - DNA, 42 bp, Artificial Sequence; identical to SEQ ID NO: 39
  Synthetic

SEQ ID NO: 84 - DNA, 45 bp, Artificial Sequence
  Synthetic
caggaggttgaggcagcagtccagcatcaccaaggtccaaatgaa

SEQ ID NO: 85 - DNA, 45 bp, Artificial Sequence
  Synthetic
aagataagcggcatggtcttgcgttggaggcgtcagactgtagcg

SEQ ID NO: 86 - DNA, 42 bp, Artificial Sequence; identical to SEQ ID NO: 42
  Synthetic

SEQ ID NO: 87 - DNA, 45 bp, Artificial Sequence
  Synthetic
atcaagtttgcctttacaccgaaagcgaggatgaggtatacacgg

SEQ ID NO: 88 - DNA, 45 bp, Artificial Sequence
  Synthetic
acctaaagctacggcagtgaccgatctccagacaaaagggcgaca

SEQ ID NO: 89 - DNA, 42 bp, Artificial Sequence; identical to SEQ ID NO: 45
  Synthetic

SEQ ID NO: 90 - DNA, 45 bp, Artificial Sequence
  Synthetic
ggtttaccagcgccaagctgtggttgtgcctcgtgagcaaatgaa

SEQ ID NO: 91 - DNA, 45 bp, Artificial Sequence
  Synthetic
aagataaggttccacaggactcgcacttcgcagatagccgaacaa

SEQ ID NO: 92 - DNA, 42 bp, Artificial Sequence; identical to SEQ ID NO: 48
  Synthetic

SEQ ID NO: 93 - DNA, 45 bp, Artificial Sequence
  Synthetic
tttttaagaaaagtaacgctcgatggcgtctcgtggtatacacgg

SEQ ID NO: 94 - DNA, 45 bp, Artificial Sequence
  Synthetic
acctaaagcaaggctacgagtcagacaggaacgtcaaaaatgaaa

SEQ ID NO: 95 - DNA, 42 bp, Artificial Sequence; identical to SEQ ID NO: 51
  Synthetic

SEQ ID NO: 96 - DNA, 45 bp, Artificial Sequence
  Synthetic
aaacgattttttgtttcgagcagtggtgagtgcggtccaaatgaa

SEQ ID NO: 97 - DNA, 31 bp, Artificial Sequence
  Synthetic
tgtatcttggtccgtgtatcttatcttgcat

SEQ ID NO: 98 - DNA, 31 bp, Artificial Sequence
  Synthetic
actcgtgtctgttcatttgctttaggtaggt

SEQ ID NO: 99 - DNA, 45 bp, Artificial Sequence; identical to SEQ ID NO: 79
  Synthetic

SEQ ID NO: 100 - DNA, 42 bp, Artificial Sequence; identical to SEQ ID NO: 36
  Synthetic

SEQ ID NO: 101 - DNA, 45 bp, Artificial Sequence; identical to SEQ ID NO: 81
  Synthetic

SEQ ID NO: 102 - DNA, 45 bp, Artificial Sequence; identical to SEQ ID NO: 82
  Synthetic

SEQ ID NO: 103 - DNA, 42 bp, Artificial Sequence; identical to SEQ ID NO: 39
  Synthetic

SEQ ID NO: 104 - DNA, 45 bp, Artificial Sequence; identical to SEQ ID NO: 84
  Synthetic

SEQ ID NO: 105 - DNA, 45 bp, Artificial Sequence; identical to SEQ ID NO: 85
  Synthetic

SEQ ID NO: 106 - DNA, 42 bp, Artificial Sequence; identical to SEQ ID NO: 42
  Synthetic

SEQ ID NO: 107 - DNA, 45 bp, Artificial Sequence; identical to SEQ ID NO: 87
  Synthetic

SEQ ID NO: 108 - DNA, 45 bp, Artificial Sequence; identical to SEQ ID NO: 88
  Synthetic

SEQ ID NO: 109 - DNA, 42 bp, Artificial Sequence; identical to SEQ ID NO: 45
  Synthetic

SEQ ID NO: 110 - DNA, 45 bp, Artificial Sequence; identical to SEQ ID NO: 90
  Synthetic

SEQ ID NO: 111 - DNA, 45 bp, Artificial Sequence; identical to SEQ ID NO: 91
  Synthetic

SEQ ID NO: 112 - DNA, 42 bp, Artificial Sequence; identical to SEQ ID NO: 48
  Synthetic

SEQ ID NO: 113 - DNA, 45 bp, Artificial Sequence; identical to SEQ ID NO: 93
  Synthetic

SEQ ID NO: 114 - DNA, 45 bp, Artificial Sequence; identical to SEQ ID NO: 94
  Synthetic

SEQ ID NO: 115 - DNA, 42 bp, Artificial Sequence; identical to SEQ ID NO: 51
  Synthetic

SEQ ID NO: 116 - DNA, 45 bp, Artificial Sequence; identical to SEQ ID NO: 96
  Synthetic

SEQ ID NO: 117 - DNA, 24 bp, Artificial Sequence
  Synthetic
tggttgctcgtgcttggctggcat

SEQ ID NO: 118 - DNA, 24 bp, Artificial Sequence
  Synthetic
tctgtgccgagcactccgtgaggt

SEQ ID NO: 119 - DNA, 24 bp, Artificial Sequence
  Synthetic
tggttcgtccgctggctctggcat

SEQ ID NO: 120 - DNA, 24 bp, Artificial Sequence
  Synthetic
tctgtcctgcgaacgcttccaggt

SEQ ID NO: 121 - DNA, 24 bp, Artificial Sequence
  Synthetic
tggttcaggcttgacggttggcat

SEQ ID NO: 122 - DNA, 24 bp, Artificial Sequence
  Synthetic
tctggcacggatcgacgctgaggt

SEQ ID NO: 123 - DNA, 54 bp, Artificial Sequence
  Synthetic
tgagtttcaaaggaacgtccaccgttttcggtggacttaactaaagatctccaa

SEQ ID NO: 124 - DNA, 54 bp, Artificial Sequence
  Synthetic
aaaaaaggcttttgcggtggtccgttttcggaccacttggatcgtcgggtagca

SEQ ID NO: 125 - DNA, 54 bp, Artificial Sequence
  Synthetic
acggctacaagtacaactcggcacttttgtgccgagttcggagattcgcgacct

SEQ ID NO: 126 - DNA, 54 bp, Artificial Sequence
  Synthetic
gctccatgacgtaacacggatcgcttttgcgatccgttaagctgctacaccaga

SEQ ID NO: 127 - DNA, 54 bp, Artificial Sequence
  Synthetic
acgagtagatcagttgcaccgctgttttcagcggtgttagatttagcgccaaaa

SEQ ID NO: 128 - DNA, 54 bp, Artificial Sequence
  Synthetic
ggaattaccaccacccgtgaggcgttttcgcctcactttcattttccgtaacac

SEQ ID NO: 129 - DNA, 54 bp, Artificial Sequence
  Synthetic
gagaataggtcaccagcggaaccgttttcggttccgtttacaaactccgccacc

SEQ ID NO: 130 - DNA, 54 bp, Artificial Sequence
  Synthetic
aaaggccgctccaaaaccgtggcgttttcgccacggttggagccttagcggagt

SEQ ID NO: 131 - DNA, 54 bp, Artificial Sequence
  Synthetic
gcgaaacaagaggcttgtgctgcgttttcgcagcactttgaggactagggagtt

SEQ ID NO: 132 - DNA, 54 bp, Artificial Sequence
  Synthetic
ccaaatcattacttagacgctggcttttgccagcgtttccggaacgtaccaagc

SEQ ID NO: 133 - DNA, 54 bp, Artificial Sequence
  Synthetic
aaagattctaaattggcgacggacttttgtccgtcgttgcttgagattcattac

SEQ ID NO: 134 - DNA, 54 bp, Artificial Sequence
  Synthetic
ctcagagcgaggcataggctccgcttttgcggagccttgtaagagcacaggtag

SEQ ID NO: 135 - DNA, 54 bp, Artificial Sequence
  Synthetic
tgtagcataactttcaggcatccgttttcggatgccttacagtttctaattgta

SEQ ID NO: 136 - DNA, 54 bp, Artificial Sequence
  Synthetic
tcggtttaggtcgctggctgacgcttttgcgtcagcttaggcttgcaaagactt

SEQ ID NO: 137 - DNA, 54 bp, Artificial Sequence
  Synthetic
tttcatgatgacccccaccagccgttttcggctggtttagcgattaaggcgcag

SEQ ID NO: 138 - DNA, 54 bp, Artificial Sequence
  Synthetic
acggtcaatgacaagacggaggcgttttcgcctccgttaccggatatggtttaa

SEQ ID NO: 139 - DNA, 54 bp, Artificial Sequence
  Synthetic
tttcaactacggaacactcgctgcttttgcagcgagttacattattaacactat

SEQ ID NO: 140 - DNA, 54 bp, Artificial Sequence
  Synthetic
cataacccaccgccacctggctcgttttcgagccagttcctcagaaacaacgcc

SEQ ID NO: 141 - DNA, 54 bp, Artificial Sequence
  Synthetic
tgctaaactccacagagccagtgcttttgcactggcttcagccctctaccgcca

SEQ ID NO: 142 - DNA, 54 bp, Artificial Sequence
  Synthetic
atatattctcagcttgccgtccgcttttgcggacggttctttcgagtgggattt

SEQ ID NO: 143 - DNA, 54 bp, Artificial Sequence
  Synthetic
ctcatcttggaagtttcggatggcttttgccatccgttccattaaacataaccg

SEQ ID NO: 144 - DNA, 54 bp, Artificial Sequence
  Synthetic
agtaatcttcataaggtctggtcgttttcgaccagattgaaccgaactaaaaca

SEQ ID NO: 145 - DNA, 54 bp, Artificial Sequence
  Synthetic
acgaactattaatcatggcacctgttttcaggtgcctttgtgaatttcatcaag

SEQ ID NO: 146 - DNA, 54 bp, Artificial Sequence
  Synthetic
ccctcagatcgtttaccgcttgcgttttcgcaagcgttcagacgacttaataaa

SEQ ID NO: 147 - DNA, 46 bp, Artificial Sequence
  Synthetic
agggatagcaagcccacaacgtgaggacacttggaggagcctacgg

SEQ ID NO: 148 - DNA, 42 bp, Artificial Sequence
  Synthetic
tgtcctcacgttgctggatgccgatcctacgacacctccaag

SEQ ID NO: 149 - DNA, 46 bp, Artificial Sequence
  Synthetic
tgctctgcctgtcgtaggatcggcatccagataggaacccatgtac

SEQ ID NO: 150 - DNA, 46 bp, Artificial Sequence
  Synthetic
gaattgcgaataataagtgaccttgctgtaccgtcgagatggagtc

SEQ ID NO: 151 - DNA, 42 bp, Artificial Sequence
  Synthetic
acagcaaggtcaccgcagttggcactaggcgacatcgacggt

SEQ ID NO: 152 - DNA, 46 bp, Artificial Sequence
  Synthetic
accacaacctgtcgcctagtgccaactgcgttttttcacgttgaaa

SEQ ID NO: 153 - DNA, 46 bp, Artificial Sequence
  Synthetic
accctcagcagcgaaacgagtacggcaacacggtgagagcctacgg

SEQ ID NO: 154 - DNA, 42 bp, Artificial Sequence
  Synthetic
gttgccgtactcgactggtcacgaacgtctccaactcaccgt

SEQ ID NO: 155 - DNA, 46 bp, Artificial Sequence
  Synthetic
tgctctgccttggagacgttcgtgaccagtgacagcatcggaacga

SEQ ID NO: 156 - DNA, 46 bp, Artificial Sequence
  Synthetic
tgtatcatcgcctgatcaacggtacgagatgcgaagcgatggagtc

SEQ ID NO: 157 - DNA, 42 bp, Artificial Sequence
  Synthetic
tctcgtaccgttgccagtagacctagccgacgtggcttcgca

SEQ ID NO: 158 - DNA, 46 bp, Artificial Sequence
  Synthetic
accacaacccacgtcggctaggtctactggaaattgtgtcgaaatc

SEQ ID NO: 159 - DNA, 46 bp, Artificial Sequence
  Synthetic
cattcagtgaataaggacgctatgcctatcgctctagagcctacgg

SEQ ID NO: 160 - DNA, 42 bp, Artificial Sequence
  Synthetic
ataggcatagcgttgctccagtctgctgctcaggctagagcg

SEQ ID NO: 161 - DNA, 46 bp, Artificial Sequence
  Synthetic
tgctctgcccctgagcagcagactggagcacttgccctgacgagaa

SEQ ID NO: 162 - DNA, 46 bp, Artificial Sequence
  Synthetic
gaataccacattcaacaccgatgaggatcacggcactgatggagtc

SEQ ID NO: 163 - DNA, 42 bp, Artificial Sequence
  Synthetic
gatcctcatcggtcaagcgaaggtgcgagcctgtagtgccgt

SEQ ID NO: 164 - DNA, 46 bp, Artificial Sequence
  Synthetic
accacaaccacaggctcgcaccttcgcttgtaatgcagatacataa

SEQ ID NO: 165 - DNA, 33 bp, Artificial Sequence
  Synthetic
caacaatcagaccgtaggctggcagagcaatgc

SEQ ID NO: 166 - DNA, 33 bp, Artificial Sequence
  Synthetic
caacaataccagactccatcggttgtggtacct

SEQ ID NO: 167 - DNA, 33 bp, Artificial Sequence
  Synthetic
gcattgctctgccagcctacggtctgattgttg

SEQ ID NO: 168 - DNA, 33 bp, Artificial Sequence
  Synthetic
aggtaccacaaccgatggagtctggtattgttg

SEQ ID NO: 169 - DNA, 46 bp, Artificial Sequence
  Synthetic
agggatagcaagcccacaacgtgaggacacttggagggacctctgg

SEQ ID NO: 170 - DNA, 42 bp, Artificial Sequence; identical to SEQ ID NO: 148
  Synthetic

SEQ ID NO: 171 - DNA, 46 bp, Artificial Sequence
  Synthetic
tccacgacttgtcgtaggatcggcatccagataggaacccatgtac

SEQ ID NO: 172 - DNA, 46 bp, Artificial Sequence
  Synthetic
gaattgcgaataataagtgaccttgctgtaccgtcgaacagagtgc

SEQ ID NO: 173 - DNA, 42 bp, Artificial Sequence; identical to SEQ ID NO: 151
  Synthetic

SEQ ID NO: 174 - DNA, 46 bp, Artificial Sequence
  Synthetic
agtcacgcttgtcgcctagtgccaactgcgttttttcacgttgaaa

SEQ ID NO: 175 - DNA, 46 bp, Artificial Sequence
  Synthetic
accctcagcagcgaaacgagtacggcaacacggtgaggacctctgg

SEQ ID NO: 176 - DNA, 42 bp, Artificial Sequence; identical to SEQ ID NO: 154
  Synthetic

SEQ ID NO: 177 - DNA, 46 bp, Artificial Sequence
  Synthetic
tccacgactttggagacgttcgtgaccagtgacagcatcggaacga

SEQ ID NO: 178 - DNA, 46 bp, Artificial Sequence
  Synthetic
tgtatcatcgcctgatcaacggtacgagatgcgaagcacagagtgc

SEQ ID NO: 179 - DNA, 42 bp, Artificial Sequence; identical to SEQ ID NO: 157
  Synthetic

SEQ ID NO: 180 - DNA, 46 bp, Artificial Sequence
  Synthetic
agtcacgctcacgtcggctaggtctactggaaattgtgtcgaaatc

SEQ ID NO: 181 - DNA, 46 bp, Artificial Sequence
  Synthetic
cattcagtgaataaggacgctatgcctatcgctctaggacctctgg

SEQ ID NO: 182 - DNA, 42 bp, Artificial Sequence; identical to SEQ ID NO: 160
  Synthetic

SEQ ID NO: 183 - DNA, 46 bp, Artificial Sequence
  Synthetic
tccacgactcctgagcagcagactggagcacttgccctgacgagaa

SEQ ID NO: 184 - DNA, 46 bp, Artificial Sequence
  Synthetic
gaataccacattcaacaccgatgaggatcacggcactacagagtgc

SEQ ID NO: 185 - DNA, 42 bp, Artificial Sequence; identical to SEQ ID NO: 163
  Synthetic

SEQ ID NO: 186 - DNA, 46 bp, Artificial Sequence
  Synthetic
agtcacgctacaggctcgcaccttcgcttgtaatgcagatacataa

SEQ ID NO: 187 - DNA, 33 bp, Artificial Sequence
  Synthetic
ttatcatcagaccagaggtcagtcgtggaatgc

SEQ ID NO: 188 - DNA, 33 bp, Artificial Sequence
  Synthetic
ttatcataccagcactctgtagcgtgactacct

SEQ ID NO: 189 - DNA, 33 bp, Artificial Sequence
  Synthetic
gcattccacgactgacctctggtctgatgataa

SEQ ID NO: 190 - DNA, 33 bp, Artificial Sequence
  Synthetic
aggtagtcacgctacagagtgctggtatgataa

SEQ ID NO: 191 - DNA, 24 bp, Artificial Sequence
  Synthetic
tggtcgatccacgcttggctgcat

SEQ ID NO: 192 - DNA, 24 bp, Artificial Sequence
  Synthetic
tggttgagcgttagtaccgagcat

SEQ ID NO: 193 - DNA, 24 bp, Artificial Sequence
  Synthetic
tggtcaccgctgcgtctccagcat

SEQ ID NO: 194 - DNA, 24 bp, Artificial Sequence
  Synthetic
tctggacactgcaccttcagaggt

SEQ ID NO: 195 - DNA, 24 bp, Artificial Sequence
  Synthetic
tctggcacgagtacaagtcgaggt

SEQ ID NO: 196 - DNA, 24 bp, Artificial Sequence
  Synthetic
tctggtctgccgagtgtccgaggt

SEQ ID NO: 197 - DNA, 24 bp, Artificial Sequence
  Synthetic
tggtcgctcacgtcctcgctgcat

SEQ ID NO: 198 - DNA, 24 bp, Artificial Sequence
  Synthetic
tggtgtcaggaacctgagtggcat

SEQ ID NO: 199 - DNA, 24 bp, Artificial Sequence
  Synthetic
tggtctccgcattgtggactgcat

SEQ ID NO: 200 - DNA, 24 bp, Artificial Sequence
  Synthetic
tctgtgttccgtgccgagtcaggt

SEQ ID NO: 201 - DNA, 24 bp, Artificial Sequence
  Synthetic
tctggcgagcaatcggatacaggt

SEQ ID NO: 202 - DNA, 24 bp, Artificial Sequence
  Synthetic
tctgcgagacacgattggcgaggt

SEQ ID NO: 203 - DNA, 24 bp, Artificial Sequence
  Synthetic
tggttcactccatgccattagcat

SEQ ID NO: 204 - DNA, 24 bp, Artificial Sequence
  Synthetic
tggtcatggcaactggttcagcat

SEQ ID NO: 205 - DNA, 24 bp, Artificial Sequence
  Synthetic
tggtcgataggagcctcatggcat

SEQ ID NO: 206 - DNA, 24 bp, Artificial Sequence
  Synthetic
tctgtggttagcacttccagaggt

SEQ ID NO: 207 - DNA, 24 bp, Artificial Sequence
  Synthetic
tctgtcaagcgatagcctcaaggt

SEQ ID NO: 208 - DNA, 24 bp, Artificial Sequence
  Synthetic
tctgagtgcgacagcgagccaggt

(19) United States
(12) Patent Application Publication
(10) Pub. No.: US 2024/0011077 A1
(43) Pub. Date: Jan. 11, 2024

(54) ACTIVATION AND INACTIVATION OF NUCLEIC ACID NANOSTRUCTURES

(71) Applicant: THE JOHNS HOPKINS UNIVERSITY, Baltimore, MD (US)

(72) Inventors: Rebecca B. Schulman, Baltimore, MD (US); Daniel Siegal, Baltimore, MD (US); Yi Li, Baltimore, MD (US); Samuel Schaffter, Baltimore, MD (US); Joanna Schneider, Baltimore, MD (US)

(21) Appl. No.: 18/247,794

(22) PCT Filed: Oct. 4, 2021
(86) PCT No.: PCT/US2021/053365

§ 371 Date: Apr. 4, 2023

Related U.S. Application Data
(60) Provisional application No. 63/180,868, filed on Apr. 28, 2021.
(60) Provisional application No. 63/087,624, filed on Oct. 5, 2020.

(51) Int. Cl. C12Q 1/682

(52) U.S. Cl. CPC C12Q 1/682

(57) ABSTRACT

Described are systems comprising nucleic acid nanostructures, e.g., nanotubes, for use in detecting biological agents. The systems comprise a nucleic acid nanotube, nucleic acid nanotube seed, a plurality nucleic acid nanotube monomers, or a combination there of; a nucleic acid nanotube terminus; and an activation strand comprising a single-stranded nucleic acid comprising a first sequence complementary to a portion of the nucleic acid nanotube, nucleic acid nanotube seed, nucleic acid nanotube monomers and a second sequence complementary to a portion of the nucleic acid nanotube terminus. Devices, kits, and methods comprising the systems are also described.

### CROSS-REFERENCE TO RELATED APPLICATIONS

This application claims the benefit of U.S. Provisional Application Nos. 63/087,624, filed Oct. 5, 2020, and 63/180,868, filed Apr. 28, 2021, the contents of which are herein incorporated by reference in their entirety.

### STATEMENT REGARDING FEDERALLY-SPONSORED RESEARCH

This invention was made with Government support under grant DE-SC0010426 awarded by the Department of Energy and under grant D16AP00147 awarded by the Defense Advanced Research Projects Agency. The Government has certain rights in the invention.

### TECHNICAL FIELD

The present disclosure relates to systems comprising nucleic acid nanostructures, e.g., nanotubes, for use in detecting biological agents.

### BACKGROUND OF THE INVENTION

Molecular assemblies inside cells often undergo structural reconfiguration in response to stimuli to alter their function. Adaptive reconfiguration of cytoskeletal networks, for example, enables cellular shape change, movement, and cargo transport and plays a key role in driving complex processes such as division and differentiation. DNA nanotubes have emerged as promising building blocks for constructing programmable synthetic analogs of cytoskeletal networks. Nucleating seeds can control when and where nanotubes grow and capping structures can bind nanotube ends to stop growth. Such seeding and capping structure, collectively called termini, can organize nanotubes into larger architectures.

### SUMMARY OF THE INVENTION

The present invention is directed to system comprising a nucleic acid nanotube, nucleic acid nanotube seed, a plurality nucleic acid nanotube monomers, or a combination there of; a nucleic acid nanotube terminus; and an activation strand comprising a single-stranded nucleic acid comprising a first sequence complementary to a portion of the nucleic acid nanotube, nucleic acid nanotube seed, nucleic acid nanotube monomers and a second sequence complementary to a portion of the nucleic acid nanotube terminus. The nucleic acid nanotube terminus may lack nucleic acid strands configured to interact with the nucleic acid nanotube, the nucleic acid nanotube seed, or the plurality of nucleic acid nanotube monomers. In some embodiments, the activation strand further comprises a toehold sequence not complementary to a portion of the nucleic acid nanotube, the nucleic acid nanotube seed, the plurality of nucleic acid nanotube monomers or the nucleic acid nanotube terminus. In some embodiments, the system further comprises an inactivation strand comprising a single-stranded nucleic acid having a sequence complementary to a portion of the activation strand. The inactivation strand may be complementary to the toehold sequence. In some embodiments, the activation and/or inactivation strands are provided as a double-stranded nucleic acid complex comprising the activation or inactivation strand hybridized to one or more partially complementary single-stranded oligonucleotides.

In some embodiments, the nanotube or nanotube seed comprises cholesterol moieties on an outer surface. In some embodiments, the nanotube or nanotube seed is embedded in and spans a lipid membrane.

The disclosure further provides, methods for detecting a biomarker of interest. In some embodiments, the methods comprise: providing a composition comprising a nucleic acid nanotube, nucleic acid nanotube seed, nucleic acid nanotube monomers, or a combination there of, and a nucleic acid nanotube terminus, wherein the nucleic acid nanotube terminus lacks nucleic acid strands configured to interact with a nanotubes, a nanotube seed, or nanotube monomers; incubating the composition with a biological sample comprising a biomarker of interest; and measuring nanotube growth. In some embodiments, the methods comprise: providing a composition comprising: a nanotube or nanotube seed comprising a capped nanotube terminus and an activation strand comprising a toehold sequence; and a vesicle comprising an encapsulated analyte, wherein the nanotube or nanotube seed is embedded in the vesicle lipid membrane; incubating the composition with a biological sample comprising the biomarker of interest; and measuring release of the analyte from the vesicle.

Other aspects of the invention will become apparent by consideration of the detailed description and accompanying drawings.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D are schematics of DNA nanotubes and the design of a nanotube terminus whose binding activity for DNA nanotubes can be specifically activated and inactivated. FIG. 1A shows DNA nanotube monomers and DNA nanotube structure. Top: DNA monomers are DAE-E double crossover molecules that self-assemble from 5 strands of synthetic DNA. Monomers consist of two DNA helices that are rigidly bound by two crossover motifs with four single-stranded sticky end regions on the two helix ends (a, b, c, and d with complementary sequences denoted by *). Each of the two types of monomers that co-assemble to form the nanotube has both an A interface and a B interface each defined by the sticky end sequences on each side of the monomer. Each monomer contains a Cy3 fluorophore (yellow) at the 5′ end of its central strand. Numbers labeling monomer domains indicate domain lengths in bases. Middle: Complementary sticky ends program lattice architecture. Bottom: A cyclized lattice forms a nanotube. Each nanotube has an A interface and a B interface each defined by the free sticky ends at each nanotube end. DNA nanotube growth can be nucleated at a DNA origami terminus. FIG. 1B shows that a DNA origami terminus is a hollow cylinder consisting of an M13 DNA scaffold folded by staple strands. The terminus nucleates DNA nanotube growth at monomer adapter structures that form a facet onto which DNA monomers can attach. There are 6 monomer attachment sites around the circumference of the terminus. An active A terminus binds to the A interface of the monomers and/or nanotubes (top panel and bottom panel). An inactive terminus is missing the adapter strands that bind to the monomer sticky ends so no nanotube nucleation can occur on them (middle panel). Termini that can be inactivated have adapter strands that each have a 5′ single-stranded region on the sticky end strands that serves as a toehold in a toehold-mediated strand displacement reaction that remove the sticky end strands. FIG. 1C is a schematic of sequential terminus activation and inactivation. FIG. 1D is a schematic of nanotube architecture reconfiguration by successive activation and inactivation of orthogonal termini that interact with a specific interface of the monomers/nanotubes.

FIG. 2 is a schematic of DNA nanotube architecture assembly and reconfiguration toolbox. Left: DNA origami seeds can nucleate nanotube growth. Middle: Capping structures that bind to nanotube ends can terminate nanotube growth. Right: Branched DNA origami seeds can be used to grow nanotubes at specific angles relative to one another.

FIGS. 3A-3D show that DNA nanotube termini can be activated and inactivated by specific DNA sequence inputs. FIGS. 3A and 3B show schematics of terminus activation (FIG. 3A) and terminus inactivation (FIG. 3B) on the left with representative fluorescence micrographs on the right. To characterize activation, inactive termini were first incubated with monomers (pre-act) and activation strands (A1a) were then added. To characterize inactivation, active termini were incubated with monomers to facilitate nanotube growth (pre-inact) and then inactivation strands (A1i) were added. The fractions of nanotubes with termini and termini with nanotubes are tabulated below. On the left side of FIG. 3C is a schematic of an experiment during which termini are sequentially activated, inactivated, then reactivated via successive additions, respectively, of activation, inactivation, and activation strands. Representative fluorescence micrographs of results are shown on the right for each stage of reconfiguration. 1×, 2×, and 4× refer to the ratio of the concentrations of the activation or inactivation strands added for a given reconfiguration step, relative to the concentration of A1a added to direct the first terminus activation. FIG. 3D are graphs of fractions of nanotubes with termini and termini with nanotubes for the experiment in (FIG. 3C). The small fraction of nanotubes attached to termini before activation in is likely the result of homogeneous nucleation followed by non-specific binding of these nanotubes to termini. Error bars represent 95% confidence intervals of proportions. Scale bars: 10 μm.

FIGS. 4A-4B are schematics of the designs of additional termini activated and inactivated by orthogonal sets of activation and inactivation strands. In FIG. 4A, the B1 terminus binds to the B interface of monomers and nanotubes. In FIG. 4B, the A2 and B2 termini that bind the A or B interfaces of monomers and nanotubes, respectively. Each terminus has unique activation strand binding sites (ASBS) and unique toehold domains on its activation strands. Numbers indicate domain lengths in bases. Letters indicate domain sequence identity with * denoting complementarity. Exemplary sequences are included in FIGS. 10-17.

FIGS. 5A-5B show that doubly terminated nanotube architectures can be assembled and disassembled by selective activation and removal of individual termini. On the right of FIG. 5A are schematics of an experiment testing the efficacy of sequential activation and inactivation of A1 and B1 termini along with representative fluorescence micrographs (right) of results after each activation or inactivation step. For clarity, all B1 termini attached to nanotubes are labeled. FIG. 5B are graphs of the fractions of nanotubes with termini and termini with nanotubes during each of the steps of the experiment in (FIG. 5A). Error bars represent 95% confidence intervals of proportions. Scale bars: 10 μm.

FIGS. 6A-B show selective reconfiguration of doubly terminated nanotube architectures. On the left side of FIG. 6A are schematics of an experiment demonstrating the stepwise transformation of an A1-B2 terminated structure to an B2-A2 terminated structure via selective terminus activation and inactivation, and fluorescence micrographs (right side) showing example results. Termini on the doubly terminated structures in the micrographs are labeled for clarity. FIG. 6B is graphs of fractions of nanotubes with termini and termini with nanotubes for the experiment in (FIG. 6A). Each stage of the experiment is initiated with the addition of the activation or inactivation strands in the respective x-axis label. Error bars represent 95% confidence intervals of proportions. Scale bars: 10 μm.

FIGS. 7A and 7B show sensitive detection of activation sequences and activation using a single activator strand sequence. FIG. 7A is a graph of fractions of termini with nanotubes after activation of the A1 terminus (A1a) or A3 terminus using unpurified (A3a) or PAGE purified activation strands (A3a purified) from IDT. The activation strands were added to the final concentrations (nM) listed above the bars as soon as the purified termini were added to the monomer mixes. The “ANL” (annealed) samples served as positive controls: these termini were annealed with their activation strands. The samples were analyzed 24 hours after the activation strands were added. Termini were at 3 μM in all samples. All termini were fluorescently labeled with atto488. Error bars represent 95% confidence intervals of proportions. FIG. 7B is a graph of the fraction of termini with nanotubes from terminus variants with different sets of presented activation strands after 25 hours of growth. The A0 terminus used six distinct to make it possible to prepare terminus variants with arbitrary combinations of sticky ends for nanotube binding. The A0 terminus did not possess the 7-base 5′ single-stranded toeholds that are used during inactivation but not during activation. The A0 terminus variants were each present at 25 μM. Monomer concentration was 45 nM. Essentially, no nucleation occurs from variant V5, where all the SE sticky ends are missing, suggesting that this variant could be used as an inactive terminus that could be activated by the addition of a single SE activation sequence. All A0 terminus variants were present at roughly 25 μM. All A0 variants were fluorescently labeled with atto647.

FIGS. 8A and 8B are RE and SE nanotube monomer designs (FIG. 8A) and respective sequences with SEQ ID NOs:1-10 (FIG. 8B). Strands 1, 2, 4, and 5 were ordered PAGE purified and strand 3 was ordered HPLC purified. Modifications marked with / / are labels as defined.

FIG. 9 is one embodiments of a design of the DNA origami termini was adopted from previous studies (Fletcher, D. A.; Mullins, R. D. Nature 2010, 463 (7280), 485-492 and Petry, S.; Vale, R. D. Nat. Cell Biol. 2015, 17, 1089-1093, incorporated herein by reference in their entireties). Each terminus is composed of a scaffold strand (M13mp18 DNA (7.240 bases)), 24 staple strands, 18 adapter strands, and 2 activation strands (strands on the adapters that present the monomer sticky end sequences). The staple strand sequences are the same as those used previously (Fletcher, D. A.; Mullins. R. D. Nature 2010, 463 (7280), 485-492, incorporated herein by reference in its entirety) and include hairpin domains that face outward after cyclization and were added to control the direction of cyclization so that the direction of curvature would match that of the nanotubes. The A and B termini are folded from different domains of the M13 DNA scaffold. To prepare A termini, the A staples and A adapter strands were mixed with Ml3 scaffold strand and annealed, while to prepare the B termini, B staples and B adapter stands were mixed with the M13 scaffold strand and annealed. The origami termini were fluorescently labeled as described previously (Blanchoin, L.; Boujemaa-Paterski. R.; Sykes. C.; Plastino, J. Physiol. Rev. 2014, 94 (1), 235-263, incorporated herein by reference in its entirety). Each labeling strand is complementary to a unique 25-base region of the unfolded M13 at its 5′ end and has a common 15-base sequence at its 3′ end. The 15-base sequence at the 3′ end of each labeling strand served as a binding site for a DNA strand that was modified with a fluorescent tag at its 5′ end (either atto488 or atto647 in this study). The labeling strands and fluorescent strand sequences are the same for both A and B termini.

FIGS. 10A and 10B are an exemplary designs of the A termini (FIG. 10A) and sequences (SEQ ID NOs: 11-34) of A termini staples (FIG. 10B). Numbers beside strand names correspond to the numbers identifying individual staples in FIG. 10A. Hairpins (underlined) were incorporated into the staples to induce a directional preference for cyclization.

FIGS. 11A and 11B are an exemplary designs of the A1 terminus adaptors (FIG. 11A) and sequences (SEQ ID NOs: 35-56) of A1 DNA origami terminus adapter strands (FIG. 11B).

FIGS. 12A and 12B are an exemplary designs of the A2 terminus adaptors (FIG. 12A) and sequences (SEQ ID NOs: 57-78) of A2 DNA origami terminus adapter strands (FIG. 12B). All of the _3 strands have the same sequences as the corresponding strands of the A1 terminus.

FIGS. 13A and 13B are an exemplary designs of the A3 terminus adaptors (FIG. 13A) and sequences (SEQ ID NOs: 79-98) of A3 DNA origami terminus adapter strands (FIG. 13B). All of the _3 strands have the same sequences as the corresponding strands of the A1 and A2 termini. The activation strands for this terminus were designed to have no predicted secondary structure.

FIGS. 14A and 14B are an exemplary designs of the A0 origami terminus adapter strands (FIG. 13A) and sequences (SEQ ID NOs: 99-122) of A0 DNA origami terminus adapter strands (FIG. 13B).

FIGS. 15A and 15B are an exemplary designs of the B termini (FIG. 15A) and sequences (SEQ ID NOs: 123-146) of B termini staples (FIG. 15B). Numbers beside strand names correspond to the numbers identifying individual staples in FIG. 15A. Hairpins (underlined) were incorporated into the staples to induce a directional preference for cyclization.

FIGS. 16A and 16B are an exemplary designs of the B1 terminus adaptors (FIG. 16A) and sequences (SEQ ID NOs: 147-168) of B1 DNA origami terminus adapter strands (FIG. 16B).

FIGS. 17A and 17B are an exemplary designs of the B2 terminus adaptors (FIG. 17A) and sequences (SEQ ID NOs: 169-190) of B2 DNA origami terminus adapter strands (FIG. 17B). All of the _3 strands have the same sequences as the corresponding strands of the B1 terminus.

FIGS. 18A and 18B show that greater than 1 nM of activation strands are required for significant activation (fraction >0.10) of the A1 terminus. FIG. 18A is representative fluorescence micrographs after activation of the A1 terminus with different activation strand concentrations. FIG. 18B are graphs of the fractions of nanotubes with termini and termini with nanotubes after activation of the A1 terminus quantified from the experiment in FIG. 18A. Error bars represent 95% confidence intervals of proportions. The activation strands (A1a) were added to the final concentrations listed on the x-axis of the plots as soon as the purified termini were added to the monomer mixes. The “Annealed” sample served as a positive control where the A1 terminus was annealed with the activation strands present. The samples were imaged after incubating 24 hours with the activation strands. A1 termini were at 3 pM for all samples.

FIGS. 19A and 19B show A1 terminus activation and inactivation using activation strands without 7-base 5′ toehold domains. Schematics of terminus activation (FIG. 19A) or terminus inactivation (FIG. 19B) are shown on the left side of each figure with representative fluorescence micrographs of results on the right side of each figure. The fractions of nanotubes with termini and termini with nanotubes are tabulated below. Pre-act refers to the incubation period before the activation strands (Ala) were added. Pre-inact refers to the incubation period before the inactivation strands (Ali) were added. Errors represent 95% confidence intervals of proportions. Scale bars: 10 μm.

FIGS. 20A and 20B show A1 terminus inactivation and reactivation. FIG. 20A has a schematic of an experiment in which termini were annealed in their active state. After nanotubes were grown from these active termini, the termini were inactivated then reactivated. Representative fluorescence micrographs at each stage of the process are shown to the right. FIG. 20B is graphs of the fractions of nanotubes with termini and termini with nanotubes for the experiment in (FIG. 20A). Error bars represent 95% confidence intervals of proportions. Scale bars: 10 μm.

FIGS. 21A-21C show nanotube joining after terminus inactivation decreases the fraction of termini with nanotubes after reactivation. FIG. 21A are graphs of fractions of nanotubes with termini and termini with nanotubes during sequential terminus inactivation and reactivation of termini with adapter sticky end strands with (A1) and without (A1o) the single-stranded inactivation toehold domains. Error bars represent 95% confidence intervals. FIG. 21B are graphs of the mean number of nanotubes and termini per field of view from the images collected for this experiment. Error bars represent standard deviation across the images. During the incubation period after terminus inactivation, the mean number of nanotubes per field of view in the sample (A1) where the termini are removed from the nanotubes during the inactivation step decreases by about a factor of two. In contrast, the mean number of nanotubes does not change in the A1o sample (termini missing the 7-base 5′ toehold domain on the activation strands) where the termini are not removed from the nanotubes by inactivation strands. This suggests that the number of nanotubes can decrease in the sample when both ends are exposed, presumably because end-to-end joining can happen in this case. The decrease in the number of nanotubes in the A1 sample post-inactivation means that fewer nanotubes are available to attach to the termini after re-activation. Given that the number of termini is the same or larger than the number of nanotubes, the decrease in the number of nanotubes available lowers the fraction of termini attached to nanotubes post-reactivation than pre-inactivation. FIG. 21C are fluorescence micrographs of samples of the reaction solution after the different stages of the experiment. Far fewer DNA nanotubes were observed in the images of the A1 sample post-inactivation than in pre-inactivation images. The nanotubes in the A1 sample post-inactivation (where the termini were removed from the nanotubes) were also visibly longer than the nanotubes in the A1o sample (where termini were not removed from the nanotubes), consistent with the end-to-end joining of nanotubes in the A1 sample during the period when the termini were inactive and both nanotube ends were free. A1o termini were present at 10.23 pM and A1 termini were as described for the experiment in FIG. 3. The A1 terminus data (top three images) is also presented in FIG. 3. Scale bars: 10 μm.

FIGS. 22A-22D show B1 terminus activation and inactivation. FIG. 22A show B1 terminus inactivation and reactivation performed in sequence on the left with representative fluorescence micrographs on the right. Initially active termini were used to grow nanotubes and subsequently inactivated by the addition of inactivation strands (B1i), then reactivated by activation strands (B1a).

FIG. 22B is graphs of the fractions of nanotubes with termini and termini with nanotubes from (FIG. 22A). Pre-inact: the incubation period before addition of inactivation strands. FIG. 22C shows successive B1 terminus activation, inactivation, and reactivation on the left with representative fluorescence micrographs from each step on the right. FIG. 22D is graphs of the fractions of nanotubes with termini and termini with nanotubes from (FIG. 22C). Pre-act: the incubation period before addition of activation strands. B1 termini attached to nanotubes are labeled for clarity. Error bars represent 95% confidence intervals of proportions. Scale bars: 10 μm.

FIGS. 23A and 23B show successive assembly, disassembly, and reassembly of an A1-B1 terminated nanotube architecture. FIG. 23A shows schematic of the experiment (left) with representative fluorescence micrographs after each step (right). FIG. 23B is graphs of fractions of nanotubes with termini and termini with nanotubes for the experiment in (FIG. 23A). Error bars represent 95% confidence intervals. Scale bars: 10 μm.

FIGS. 24A-24C are the predicted secondary structure of activation strands for A1 and A2 termini (FIG. 24A). B1 and B2 termini (FIG. 24B), and for an A3 terminus that was specifically designed so that its activation strands would not have secondary structure (FIG. 24C). Secondary structures were predicted using NUPACK5 with the default settings at 20° C. Color coding of the bases represents the probability that a given base will be in the depicted conformation at equilibrium. The gray dashed lines labeled ASBS* are the domains of activation strands that hybridize to the activator strand binding sites (ASBS) on the termini. For the A1. A2, B1, and B2 termini, the ASBS* domains all have some predicted structure which could lower their binding affinity for their target termini.

FIGS. 25A and 25B show that the activation strands designed to have no secondary structure can activate termini at much lower concentrations than activation strands with significant amounts of predicted secondary structure. FIG. 25A is graphs of the fractions of nanotubes with termini and termini with nanotubes after activation of the A1 terminus (Ala) or A3 terminus using unpurified (A3a) or PAGE purified activation strands (A3a purified) from IDT. Error bars represent 95% confidence intervals of proportions. The activation strands were added to the final concentrations listed on the x-axes of the plots as soon as the purified termini were added to the monomer mixes. The “ANL” samples served as positive controls: these termini were annealed with their activation strands present. The samples were imaged after incubating them for 24 hours with the activation strands. The fraction of termini with nanotubes is also presented in FIG. 7. FIG. 25B is graphs of the fractions of nanotubes with termini and termini of the samples activated 0.1 nM of activation strands after 24 and 115 hours. The fraction of A3 termini that are activated increases significantly with longer incubation time. Error bars represent 95% confidence intervals of proportions. Termini were at 3 pM across the samples. All termini fluorescently labeled with atto488.

FIG. 26 is a schematic of a nanoscale channel for molecular transport based on DNA nanotubes. The channel has an internal diameter of 4-6 nm and a length that can extend for multiple microns. The channels were composed of a DNA origami pore that penetrates and spans a lipid membrane and a DNA nanotube self-assembled from DAE-E double crossover tiles, consisting of five DNA oligomers that grow from the DNA origami pore.

FIG. 27 is a schematic of DNA nanotube channel to transport an agent across a lipid bilayer.

FIG. 28 shows DNA origami pores, with or without attaching to DNA nanotubes, formed channels across lipid membrane after modified with 12 cholesterol moieties. TEM images (middle) and show DNA seed pores and nanotubes inserted into the lipid membrane of small unilamellar vesicles (SUVs) and fluorescent images (right) show DNA seed pores and nanotubes inserted into the lipid membrane of giant unilamellar vesicles (GUVs).

FIG. 29 shows characterization of molecular transport through the DNA nanotube channels as a result of DNA channels spontaneously inserting into GUVs. The fluorescent dyes, initially present only outside of GUVs, were transported into GUVs through the channels penetrating the lipid membranes resulting in increased fluorescent intensity inside GUVs (left). The influx process, which takes hours to complete, was observed with a fluorescence confocal microscope (right).

FIGS. 30A-30B are graphs of transport rates of rhodamine molecules through DNA pores quantified by time-lapse measurement of fluorescent intensity inside GUVs.

FIGS. 31A-31B show a DNA origami cap (FIG. 31A) capable of specifically binding to seed pores stopped transport through the pores, as indicated by reduced influx rates (FIG. 31B).

FIGS. 32A-32C are graphs of transport rates through the DNA nanotube channels. Transport rates were longer compared to those through DNA pores, due to longer diffusion distances, and were widely distributed as nanotube lengths vary (FIG. 32C).

FIGS. 33A-33B shows an exemplary sensor technology. A nanopore embedded in a giant unilamellar vesicle membrane can be opened as a result of a specific chemical molecule (e.g., DNA sequence) facilitating removal of DNA cap on the nanotube thus releasing a large amount of an output molecule (e.g., glucose) or molecules (e.g., ATP and luciferin as in FIG. 33B) into the surrounding solution allowing detection (FIG. 33A).

FIG. 34A shows the step-wise assembly of hierarchical nanostructures via terminus inactivation. In vial 1, A1-B1 doubly terminated nanotubes were grown from A1 termini and capped with B1 termini. In vial 2, “Y” DNA nanotube structures were grown from Y-shaped DNA origami termini an A interface on all three arms (Ay). The doubly terminated nanotubes in Vial 1 were mixed in a 12:1 ratio with the Y nanotube structures in Vial 2 and the A1 termini were inactivated to allow end-to-end joining between B1 terminated nanotubes and Y nanotube structures (Right-side). The yellow box encloses an example target structure where a Y nanotube architecture has joined to a B1 terminated nanotube on all three arms. A2 and B1 termini are labeled in micrographs for clarity. Scale bars: 10 μm.

FIG. 34B shows the sequences of the AY activation strands.

FIG. 35A is a fluorescence microscopy image showing opening of nanopores embedded in a vesicle with 1 μM of a DNA uncapping strand resulting in release of the fluorescence dye TAMRA from within the vesicle lumen. FIG. 35B is a graph of the fraction of nanopores uncapped by treatment with varying amounts of the DNA uncapping sequence.

FIG. 36 is a graph of the calibrated glucose concentration released from vesicles comprising a capped nanopore as measured with an over-the-counter glucometer upon treatment with varying concentrations of an uncapping strand or a random sequence controls strand.

### DETAILED DESCRIPTION OF THE INVENTION

Disclosed herein is a system and methods directed to the selective regulation of the binding affinity of nanotube termini for nanotube monomers or nanotubes in the reconfiguration of nanotube architectures. Using nucleic acid hybridization and strand displacement reactions that specifically activate or inactivate four orthogonal nanotube termini, nanotube architectures can be reconfigured by selective addition or removal of unique termini. Terminus activation can act as a sensitive detector and amplifier of a biomarker signal.

The systems described herein allow nanotube activation to be triggered in response to sub-nanomolar concentrations of a single activation sequence, resulting in the growth of micron-scale structures from nanoscale structures, allowing isothermal and enzyme free detection of nucleic acids. For example, short, 16-base DNA sequences were detected using the systems and methods described herein. The detection was monitored with fluorescence, however, other methods known in the art including labeling nanotube monomers with plasmonic nanoparticles such that assembly would trigger an optical chance for naked eye detection can also be used. Preferably the detection method would be amenable to a single pot system and method, as described herein.

If nanotubes were used as channels or tracks for transportation of molecular cargo, dynamic rearrangement of nanotube connections between termini anchored at different points on a surface or on different objects could be used to regulate when and where signals or materials are transported.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s).” “having,” “has.” “can,” “contain(s).” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Complementarity” or “complementary to,” as used herein, refer to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary).

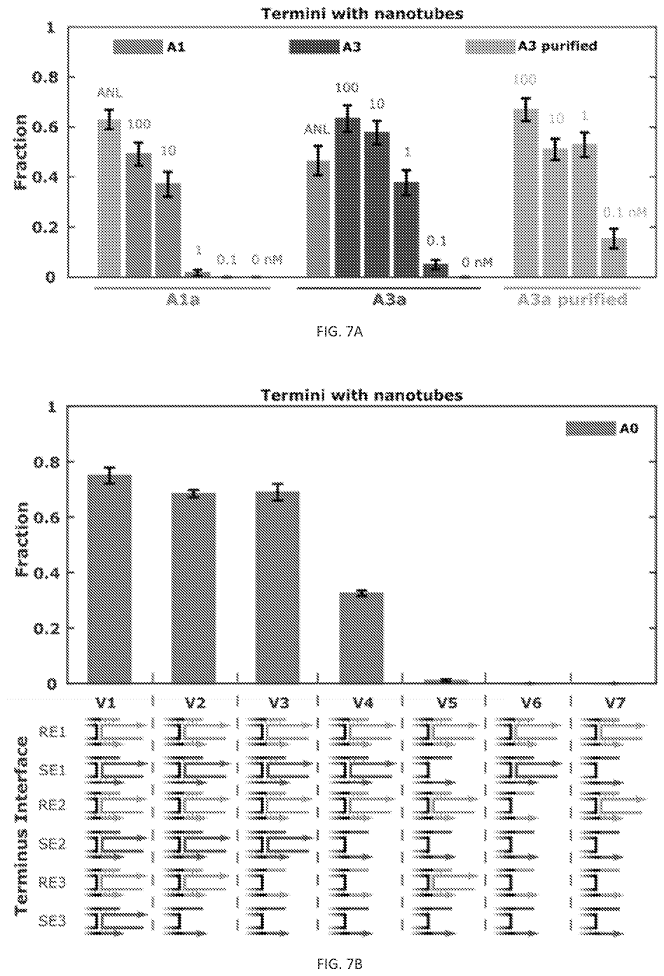

As used herein, “DNA nanotube” refers to a structure composed of individual units that form from five short DNA strands that self-assemble to form a rigid, brick-like structure, called a monomer (FIG. 6A) because of a preference for Watson-Crick complementarity. The sequence of these strands is random, non-genetic, and usually, made synthetically. Each of these basic units, or monomers has four locations where they join to similar sites on other monomers (FIG. 6B) in a pattern that allows them to assemble to form a repeating structure, or polymer (FIGS. 6C & D). This growth process can happen at the same conditions that cells grow at, to allow nanotubes to grow concomitantly with the cells. The specificity of DNA hybridization and the predictable sequence-independent structure of the DNA double helix has enabled assembly of DNA nanotubes while controlling structure, circumference, and length, as well as functionalization to connections to a variety of other materials. See, for example, Rothemund. P. W. K., et al., J. Am. Chem. Soc. 2004, 126, 16344-16352. Liu D. et al., Proc. Natl. Acad. Sci. U.S.A., 2004 101 (3) 717-722, Aldaye. F. A., et al., Nat. Nanotechnol. 2009, 4, 349-352, Mitchell, J. C., et al., J. Am. Chem. Soc. 2004, 126, 16342-16343, Wilner. O. I., et al., Nat. Commun. 2011, 2, 540., Liu H. et al, Angew Chem Int Ed Engl. 2006 Mar. 13; 45(12):1942-5, Yin, P., et al., Science 2008, 321, 824-826, Douglas, S. M., et al., Proc. Natl. Acad. Sci. U.S.A. 2007, 104, 6644-6648. Aldaye. F. A., et al., Science 2008, 321, 1795-1799. Bui, H., et al., Nano Lett. 2010, 10, 3367-3372, Sharma, J. et al., Science 2009, 323, 112-116. Shen, X., et al., J. Am. Chem. Soc. 2011, 134, 146-149. Mohammed, A. M. and Schulman. R. Nano Lettern 13 (9) 4006-4013, 2013, Mohammed. A. M., et al., Nature Nanotechnology 12, 312-316, 2017, and Mohammed, A. M., et al., Nanoscale, 9, 522-526, 2017, all of which are incorporated herein by reference. Any of these methods, as well as those described elsewhere in the specification, may be used to form the DNA nanotubes used herein. In some embodiments, nanotubes can be assembled using the monomers formed by RNA strands or RNA/DNA strands. These monomers will assemble RNA nanotubes or RNA/DNA hybrid nanotubes, see, for example, Stewart J. M. et al. ACS Nano 2019, 13, 5, 5214-5221. Agarwal S. et al, J. Am. Chem. Soc. 2019, 141, 19, 7831-7841. Thus. “nucleic acid nanotube” refers herein to nanotubes formed from DNA strands. RNA strands or RNA/DNA strands. In some embodiments, nanotubes can be assembled using process of nucleation from short nanotube segments, referred to as nanotube seeds. Nanotubes seeds can facilitate growth by the addition of DNA tile nanotubes or monomers. DNA nanotube monomers (also referred to as DNA tiles) hybridize to nanotube seeds and nanotubes via complementary single-stranded DNA ‘sticky ends,’ resulting in nanotube lengthening. See Mohammed, A. M., et al., Nature Nanotechnology 12, 312-316, 2017 and Mohammed, A. M., et al., Nanoscale, 9, 522-526, 2017, Jorgenson T. D., ACS Nano, 17, 1927-1936, 2017 incorporated herein by reference.

“Hybridization” refers to a reaction in which one or more polynucleotides react to form a complex that is stabilized via hydrogen bonding between the bases of the nucleotide residues. The hydrogen bonding may occur by Watson Crick base pairing. Hoogstein binding, or in any other sequence specific manner according to base complementarity. The complex may comprise two strands forming a duplex structure, three or more strands forming a multi stranded complex, a single self-hybridizing strand, or any combination of these. A second sequence that is complementary to a first sequence is referred to as the “complement” of the second sequence. The term “hybridizable” as applied to a polynucleotide refers to the ability of the polynucleotide to form a complex that is stabilized via hydrogen bonding between the bases of the nucleotide residues in a hybridization reaction.

“Polynucleotide” or “oligonucleotide” or “nucleic acid.” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine, and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

Any compositions or methods provided herein can be combined with one or more of any of the other compositions and methods provided herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### 2. Systems and Compositions

The present disclosure provides systems and compositions comprising a nucleic acid nanotube, nucleic acid nanotube seed, a plurality nucleic acid nanotube monomers, or a combination there of; a nucleic acid nanotube terminus; and an activation strand comprising a single-stranded nucleic acid comprising a first sequence complementary to a portion of the nucleic acid nanotube, nucleic acid nanotube seed, nucleic acid nanotube monomers and a second sequence complementary to a portion of the nucleic acid nanotube terminus.

A nucleic acid nanotube terminus lacks nucleic acid strands at a single end or both ends configured to interact with the nucleic acid nanotube, the nucleic acid nanotube seed, or the plurality of nucleic acid nanotube monomers. For example, growth of nanotubes from a nanotube seed or existing nanotube occurs due to hybridization of complementary single stranded nucleic acids with nanotube monomers. A nanotube terminus does not include available single stranded nucleic acids with which nanotube monomers would hybridize.

In some embodiments, the nanotube terminus comprises activation strand binding sites comprising nucleic acid sequences complementary to the second sequence of the activation strand. Thus, the activation strand provides in its first sequence a single-stranded nucleic acid sequence capable of binding to a nanotube, nanotube seed or nanotube monomers. The activation strand binding sites may be the same or different at either end of the nanotube or individually between different activation strand binding sites. For example, the nanotube terminus may comprise two different activation strand binding sites.

In some embodiments, the nanotube or nanotube seed, nanotube terminus, and the activation strand, are linked as one continuous structure as the activation strand hybridizes with each of the nanotube or nanotube seed and the nanotube terminus. In some embodiments, the structure comprises a nanotube liked to more than one nanotube terminus, for example, a terminus at each end of the tube.

In some embodiments, the nanotube terminus comprises a capped end. A capped end does not include activation strand binding sites and is unable to be extended by a nanotube, nanotube seed or nanotube monomers. Thus, a cap terminates nanotube growth. In some embodiments, a nanotube may comprise two capped nanotube termini at either end of the tube, thus creating an internal chamber within the nanotube.

The activation strand is a single-stranded nucleic acid comprising a first sequence complementary to a portion of the nucleic acid nanotube or nanotube seed and a second sequence complementary to a portion of the nucleic acid nanotube terminus. The first sequence may comprise two separate sequences separate by bases which do not interact with a nanotube or nanotube seed. In some embodiments, the is two separate sequences flanking the second sequence.

The activation strand may be any length necessary to allow interaction with both the nanotube or nanotube seed and the nanotube terminus which is variable and dictated by the design of the nanotube. The activation strand may be greater than about 14 bases. In some embodiments, the activation strand comprises 14-50 bases. In exemplary embodiments, the activation strand comprises 30-35 bases. The region complementary to the nanotube or nanotube seed may be on the 3′ or the 5′ end.

Commonly single-stranded nucleic acids adopt second structures, for example, stems, loops, and the like. In some embodiments, the activation strand does not adopt any significant or substantial secondary structures. The likely occurrence of secondary structure(s) within a nucleic acid sequence can be predicted through computational modeling known in the art, for example Nucleic Acid Package (NUPACK) (Zadeh. JN. et al., Journal of Computational Chemistry (2011), Vol. 32(1), 170-173).

In some embodiments, the system comprises less than 1 nM of the activation strand. The system may comprise less than 1 nM, less than 0.75 nM, less than 0.5 nM, less than 0.1 nM, or less than 0.01 nM of the activation strand.

The activation strand may further comprise a toehold sequence. The toehold sequence is a single-stranded sequence not complementary to either a portion of the nanotube, nanotube seed, or nanotube monomers. Thus, the toehold sequence does not participate in activating nanotube growth from the terminus. The toehold sequence may comprise any sequence of any length which allows it to maintain single-stranded and not interfere with nanotube growth following binding to the nanotube terminus. In some embodiments, the toehold sequence is at least 4-base single-stranded sequence at the end of the activation strand. In some embodiments, the toehold sequence is at least 7-base single-stranded sequence at the end of the activation strand. The toehold sequence may be between 4 and 10 bases.

The system may further comprise an inactivation strand. The inactivation strand is a single-stranded nucleic acid having a sequence complementary to a portion of the activation strand. In some embodiments, the inactivation strand is complementary to the toehold sequence. In some embodiments, the inactivation strand is complementary to the toehold sequence and at least a portion of the first and second sequences of the activation strand. The inactivation strand is configured to bind to the activation strand, and through a strand displacement reaction, disassemble the terminus-nanotube structure by removing the activation strand essentially linking the two components.

The inactivation strand may be any length necessary to facilitate the toehold strand displacement reaction, usually at least about 5 bases longer than the activation strand. In some embodiments, the inactivation strand comprises 20-50 bases. In exemplary embodiments, the inactivation strand comprises 30-35 bases. Alternatively, the inactivation strand can be greater than 50 bases, providing at least a portion of the inactivation strand is complementary to the toehold sequence.

In some embodiments, the activation and inactivation strands are included in a double-stranded nucleic acid complex comprising the activation or inactivation strand hybridized to one or more partially complementary single-stranded oligonucleotides. See for example Fern. J., et al., Nat. Commun. 2018, 9 (1), 3766 and Seelig, G., et al., Science 2006, 314 (5805), 1585-1588, each incorporated herein by reference in their entirety.

In some embodiments, the system further comprises a biological sample. The sample can be any suitable sample obtained from any suitable subject, typically a mammal (e.g., dogs, cats, rabbits, mice, rats, goats, sheep, cows, pigs, horses, non-human primates, or humans). Preferably, the subject is a human. The sample may be obtained from any suitable biological source, such as, a nasal swab or brush, or a physiological fluid including, but not limited to, whole blood, serum, plasma, interstitial fluid, saliva, ocular lens fluid, cerebral spinal fluid, sweat, urine, milk, ascites fluid, mucous, synovial fluid, peritoneal fluid, vaginal fluid, menses, amniotic fluid, semen, feces and the like.

The sample can be obtained from the subject using routine techniques known to those skilled in the art, and the sample may be used directly as obtained from the biological source or following a pretreatment to modify the character of the sample or extract nucleic acids from the sample. Such pretreatment may include, for example, preparing plasma from blood, diluting viscous fluids, filtration, precipitation, dilution, distillation, mixing, concentration, inactivation of interfering components, the addition of reagents, lysing, and the like.

The biological sample may comprise a biomarker configured to interact with the double-stranded nucleic acid complex facilitating release of the activation or inactivation strand. A biomarker, as used herein, is a molecule of which the presence in a biological sample from a subject indicates the presence of a disease or disorder, the risk of a disease or disorder, an infection, a microbiological agent in the sample or subject, or an environmental exposure of the subject. The biomarker may include polypeptide, glycoprotein, nucleic acid, small molecule, carbohydrate, lipid, hormone, etc.

In some embodiments, the biomarker is a nucleic acid (e.g., free circulating nucleic acids, messenger RNAs, non-coding RNAs (e.g., miRNAs)). In some embodiments, the biomarker is a nucleic acid from a pathogenic agent (e.g., a bacterium, a virus, a parasite, a viroid, a fungus, and the like).

In some embodiments, the biomarker comprises a nucleic acid strand comprising a sequence complementary to the one or more partially complementary single-stranded oligonucleotides.

In some embodiments, the nanotube or nanotube seed is embedded in and spans a lipid membrane. The nanotube or nanotube seed may comprise cholesterol moieties on the outer surface of the nanotube or seed to facilitate membrane insertion. The lipid membrane may be the lipid membrane of a vesicle. The vesicle may comprise any structure consisting of an encapsulated liquid enclosed by a lipid membrane. The vesicle may be an engineered vesicle, a cell, or a vesicular structure within a cell.

In some embodiments, the vesicle is a unilamellar vesicle (e.g., a small unilamellar vesicle (SUV) or a giant unilamellar vesicle (GUV)). Unilamellar vesicles are spherical vesicles, bounded by a single bilayer of an amphiphilic lipid or a mixture of such lipids, containing an aqueous solution inside the vesicle chamber. Roughly, small unilamellar vesicles (SUV) are typically below 100 nm, large unilamellar vesicles (LUV) are between 100 nm and 1 μm, and giant unilamellar vesicles (GUV) are above 1 μm. Unilamellar vesicles can be prepared by a variety of methods including, but not limited to, extrusion, detergent dialysis, in the case of LUVs or GUVs, fusion of SUV, reverse evaporation, and ethanol injection.

In some embodiments, the vesicle encapsulates an analyte. An analyte comprises any substance that can be measured or analyzed directly or indirectly. For example, directly measured analytes include, but are not limited to: dye molecules and moieties (e.g., fluorescent moieties, visible dyes. UV detectable molecules, and the like). Indirectly measured analytes include small molecules (e.g., glucose, lactate, neurotransmitters, luciferin) that interact with an additional agent to produce a measurable signal. For example, glucose interacts with glucose oxidase to produce measurable signal (electrochemical or visible), or luciferin interacts with luciferase and ATP to emit light.

### 3. Methods

The present disclosure provides a system which can be used to reconfigure and reorganize nucleic acid nanostructures. The system is useful in methods of detecting biomarkers of interest. The present disclosure provides methods for detecting a biomarker of interest.

In some embodiments, the methods comprise providing a composition comprising a nucleic acid nanotube, nucleic acid nanotube seed, nucleic acid nanotube monomers, or a combination there of, and a nucleic acid nanotube terminus, wherein the nucleic acid nanotube terminus lacks nucleic acid strands configured to interact with a nanotubes, a nanotube seed, or nanotube monomers; incubating the composition with a biological sample comprising a biomarker of interest; and measuring nanotube growth.

The composition may comprise varying amounts of the components of the composition. The composition may comprise between 1 pM and 1 nM of the nanotube terminus. In some embodiments, the composition comprises between 1 pM and 100 pM, between 1 pM and 75 pM, between 1 pM and 50 pM, between 1 pM and 25 pM, between 1 pM and 10 pM of the nanotube terminus.

The compositions may further comprise a double-stranded nucleic acid complex comprising an activation strand hybridized to one or more partially complementary single-stranded oligonucleotides, wherein the activation strand is a single-stranded nucleic acid comprising a first sequence complementary to a portion of the nucleic acid nanotube, nucleic acid nanotube seed, nucleic acid nanotube monomers, and a second sequence complementary to a portion of the nucleic acid nanotube terminus. The biomarker of interest may interact with the double stranded nucleic acid to release the activation strand.

In some embodiments, the biomarker of interest may be a nucleic acid biomarker. In some embodiments, the biomarker of interest may be a single-stranded nucleic acid biomarker. In some embodiments, the biomarker of interest is a nucleic acid from a pathogenic agent (e.g., a bacterium, a virus, a parasite, a viroid, a fungus, and the like).

The nucleic acid biomarker may hybridize with the nucleic acid nanotube terminus and the nanotube, nanotube seed, or nanotube monomers to induce nanotube growth. Alternatively, the nucleic acid biomarker, may release the activation strand by, for example, hybridizing to the partially complementary single stranded oligonucleotides in the double-stranded nucleic acid thereby releasing the activation strand, which then hybridizes with the nucleic acid nanotube terminus and the nanotube, nanotube seed, or nanotube monomers to induce nanotube growth. The biomarker nucleic acid may comprise 10-50 bases. The biomarker nucleic acid may comprise 10-15 bases, 10-20 bases, 10-25 bases, 10-30 bases, 10-35 bases, 10-40 bases, 10-45 bases, 15-20 bases, 15-25 bases, 15-30 bases, 15-35 bases, 15-40 bases, 15-45 bases, 15-50 bases, 20-25 bases, 20-30 bases, 20-35 bases, 20-40 bases, 20-45 bases, 20-50 bases, 25-30 bases, 25-35 bases, 25-40 bases, 25-45 bases, 25-50 bases, 30-35 bases, 30-40 bases, 30-45 bases, 30-50 bases, 35-40 bases, 35-45 bases, 35-50 bases, 40-45 bases, 40-50 bases, or 45-50 bases. In some embodiments, the biomarker of interest may comprise 30-35 bases.

In some embodiments, the methods comprise providing a composition comprising: a nanotube or nanotube seed comprising a capped nanotube terminus and an activation strand comprising a toehold sequence; and a vesicle comprising an encapsulated analyte, wherein the nanotube or nanotube seed is embedded in the vesicle lipid membrane; incubating the composition with a biological sample comprising the biomarker of interest; and measuring release of the analyte from the vesicle. In some embodiments, the nanotube or nanotube seed further comprises cholesterol moieties on an outer surface for facile entry into the vesicle membrane.

The activation strand may further comprise a toehold sequence. The toehold sequence is a single-stranded sequence not complementary to either a portion of the nanotube, nanotube seed, or nanotube monomers. Thus, the toehold strand does not participate in activating nanotube growth from the terminus and is exterior to the main nanotube structure. In some embodiments, the toehold sequence is at least 7-base single-stranded sequence at the 5′ end of the activation strand.

The compositions may further comprise a double-stranded nucleic acid complex comprising an inactivation strand hybridized to one or more partially complementary single-stranded oligonucleotides, wherein the inactivation strand is complementary to the toehold sequence. In some embodiments, the inactivation strand is complementary to the toehold sequence and at least a portion of the first and second sequences of the activation strand. The inactivation strand is configured to bind to the activation strand, and through a strand displacement reaction, disassemble the terminus-nanotube structure by removing the activation strand essentially linking the two components.

The inactivation strand may be any length necessary to facilitate the toehold strand displacement reaction. In some embodiments, the inactivation strand comprises 10-50 bases. In exemplary embodiments, the inactivation strand comprises 30-35 bases. Alternatively, the inactivation strand can be greater than 50 bases, providing at least a portion of the inactivation strand is complementary to the toehold sequence.

In some embodiments, the biomarker of interest is a single-stranded nucleic acid. In some embodiments, the single stranded nucleic acid biomarker is complementary to the toehold sequence. In some embodiments, the single-stranded nucleic acid is complementary to the toehold sequence and at least a portion of the first and second sequences of the activation strand. In some embodiments, the biomarker of interest is a nucleic acid from a pathogenic agent (e.g., a bacterium, a virus, a parasite, a viroid, a fungus, and the like).

The nucleic acid biomarker may interact with the double stranded nucleic acid to release the activation strand. The sample may comprise less than 100 nM of the single stranded nucleic acid biomarker. In some embodiments, the sample comprises less than 75 nM, less than 50 nM, less than 25 nM, less than 10 nM, less than 1 nM, less than 0.75 nM, less than 0.5 nM, less than 0.25 nM, less than 0.1 nM of the single-stranded nucleic acid biomarker.

The composition may comprise varying amounts of the components of the composition. The composition may comprise between 1 pM and 1 nM of the nanotube terminus. In some embodiments, the composition comprises between 1 pM and 750 pM, 1 pM and 500 pM, 1 pM and 250 pM, 1 pM and 100 pM, between 1 pM and 75 pM, between 1 pM and 50 pM, between 1 pM and 25 pM, between 1 pM and 10 pM of the nanotube terminus.

In some embodiments, the vesicle is a unilamellar vesicle (e.g., a small unilamellar vesicle (SUV) or a giant unilamellar vesicle (GUV)). Unilamellar vesicles are spherical vesicles, bounded by a single bilayer of an amphiphilic lipid or a mixture of such lipids, containing an aqueous solution inside the vesicle chamber. Roughly, small unilamellar vesicles (SUV) are typically below 100 nm, large unilamellar vesicles (LUV) are between 100 nm and 1 μm, and giant unilamellar vesicles (GUV) are above 1 μm. Unilamellar vesicles can be prepared by a variety of methods including, but not limited to, extrusion, detergent dialysis, in the case of LUVs or GUVs, fusion of SUV, reverse evaporation, and ethanol injection.

In some embodiments, the vesicle encapsulates an analyte. An analyte comprises any substance that can be measured or analyzed directly or indirectly. For example, directly measured analytes include, but are not limited to: dye molecules and moieties (e.g., fluorescent moieties, visible dyes, UV detectable molecules, and the like). Indirectly measured analytes include small molecules (e.g., glucose, lactate, neurotransmitters, luciferin) that interact with an additional agent to produce a measurable signal. For example, glucose interacts with glucose oxidase to produce measurable signal (electrochemical or visible), or luciferin interacts with luciferase and ATP to emit light. In some embodiments, the analyte comprises a dye, a small molecule detectable agent, a detectable moiety, or a combination thereof. The dye may comprise a fluorescent dye, a UV dye, or a visible dye. In some embodiments, the composition further comprises a moiety in a solution surrounding the vesicles configured to interact with the analyte and produce a measurable signal.

Descriptions of the nanotube, nanotube seed, nanotube terminus, capped terminus, activation and inactivation strands, toehold sequence, biomarkers, vesicle and other components of the methods set forth above for the disclosed system are also applicable to the methods described herein.

### 4. Devices

The present disclosure also provides devices comprising the systems or compositions described herein. It is understood that the disclosed devices can be employed in connection with the disclosed methods. The devices may comprise sensors for measuring a biomarker in a point-of-care or laboratory setting.

### 5. Kits

Also within the scope of the present disclosure are kits that include the components of the present systems.

The kits may comprise a nucleic acid nanotube, nanotube seed, nanotube monomers, and nanotube terminus. The kit may further comprise at least one or all of: activation/inactivation strands, analytes, vesicles, and detection moieties. Any of the components in the kit may further comprise a fluorescent tag or label, or alternatively, the kit may further comprise a fluorescent tag or label configured to label any of the components of the kits. The descriptions of the components of the kit provided elsewhere in the disclosure are also relevant here.

Individual member components of the kits may be physically packaged together or separately. The components of the kit may be provided in bulk packages (e.g., multi-use packages) or single-use packages. The kits can also comprise instructions for using the components of the kit. The instructions are relevant materials or methodologies pertaining to the kit. The materials may include any combination of the following: background information, list of components and their availability information (purchase information, etc.), brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods, compositions or systems disclosed herein. The kits optionally may provide additional components such as buffers and disposable single-use equipment (e.g., pipettes, cell culture plates or flasks).

The kits provided herein are in suitable packaging. Suitable packaging includes, but is not limited to, vials, bottles, jars, flexible packaging, and the like.

### 6. EXAMPLES

### Materials and Methods

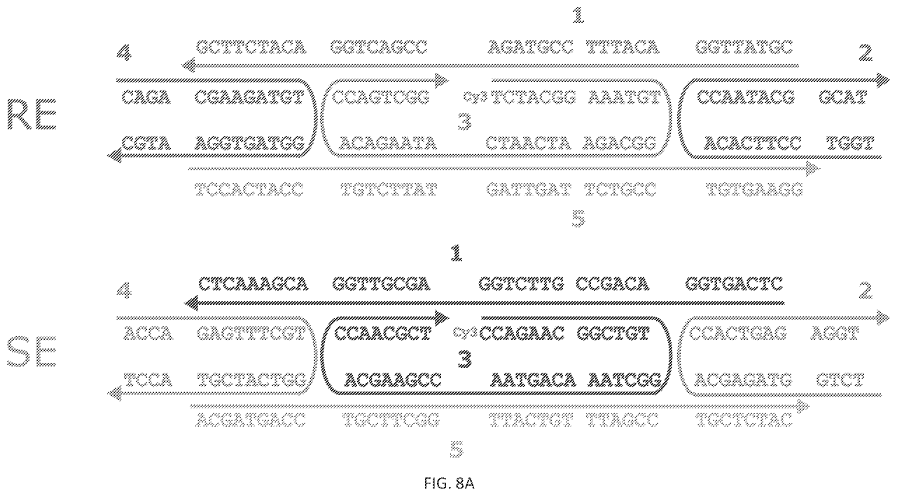

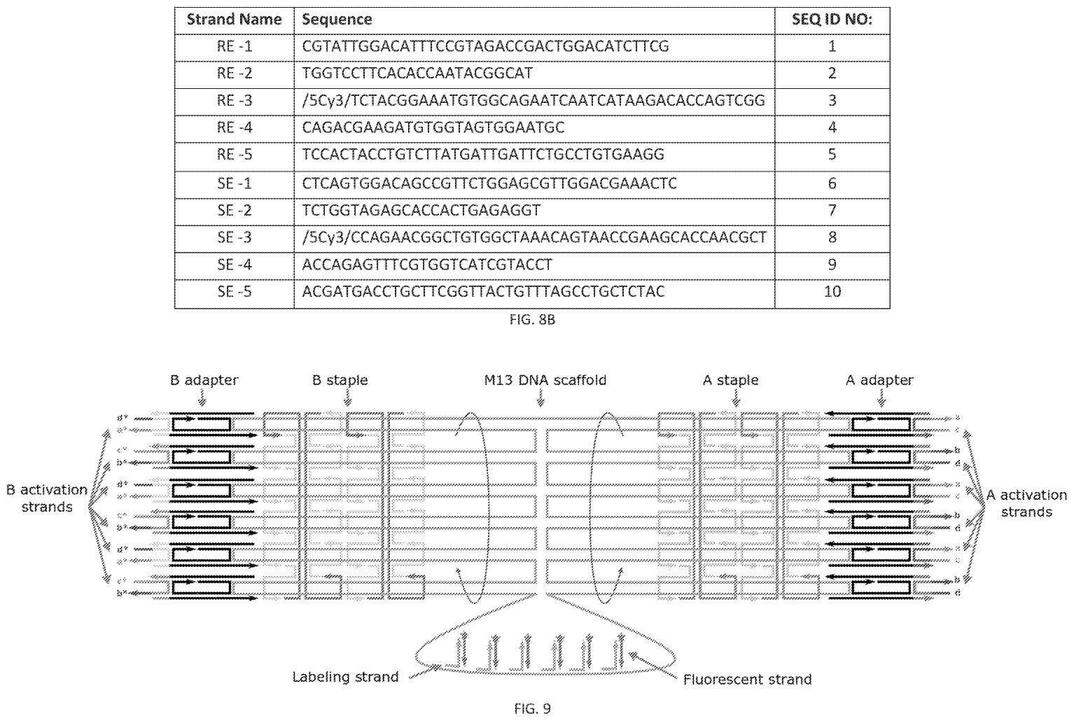

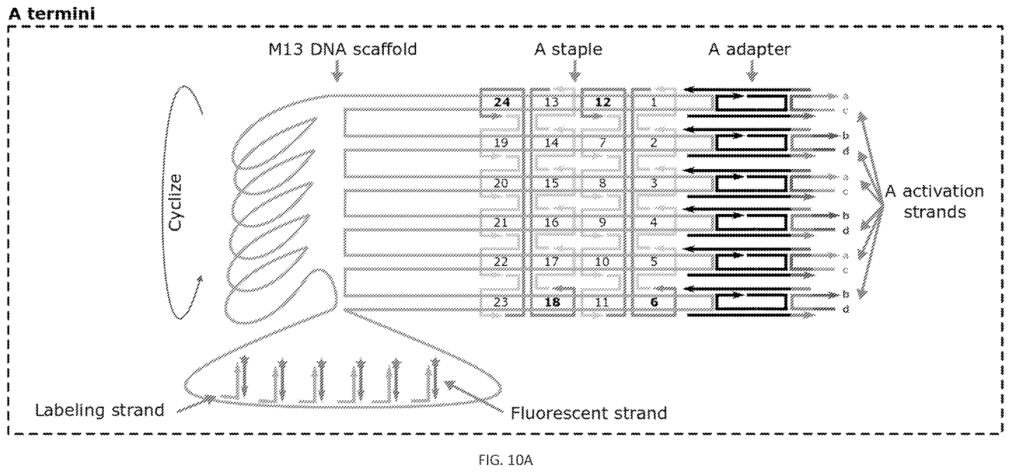

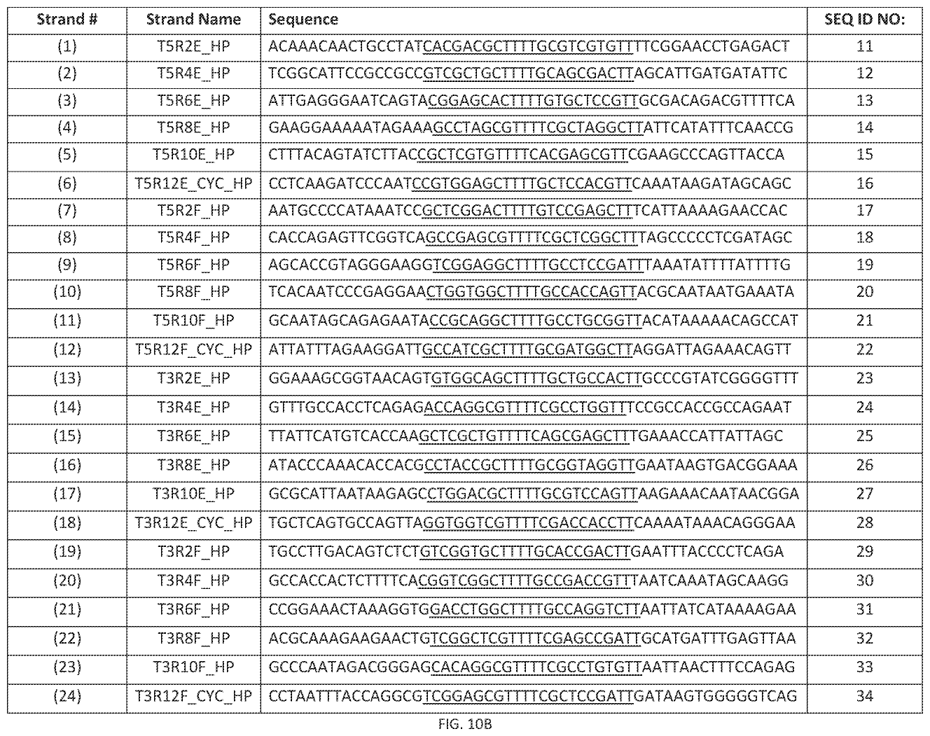

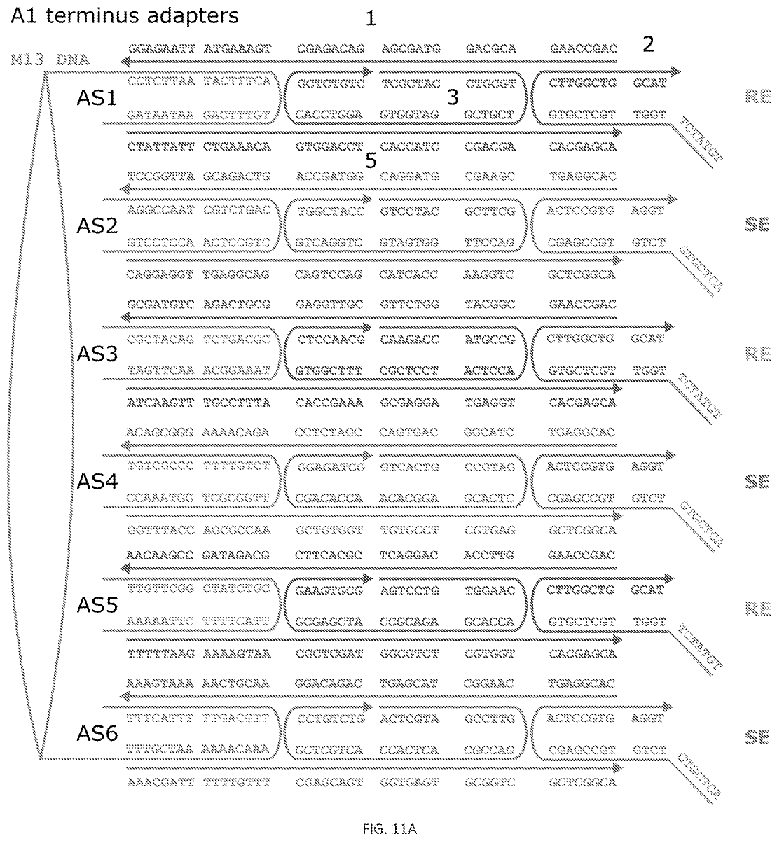

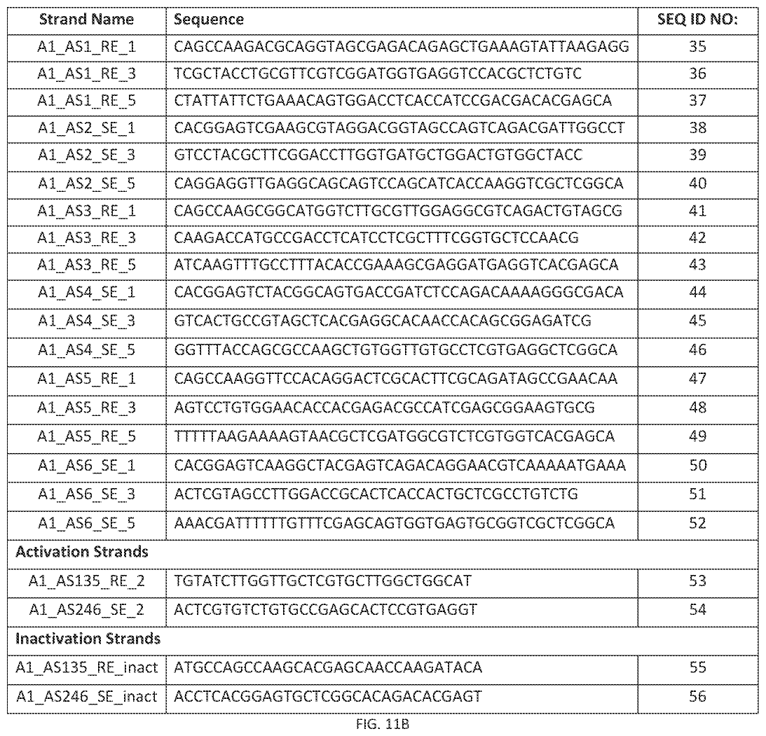

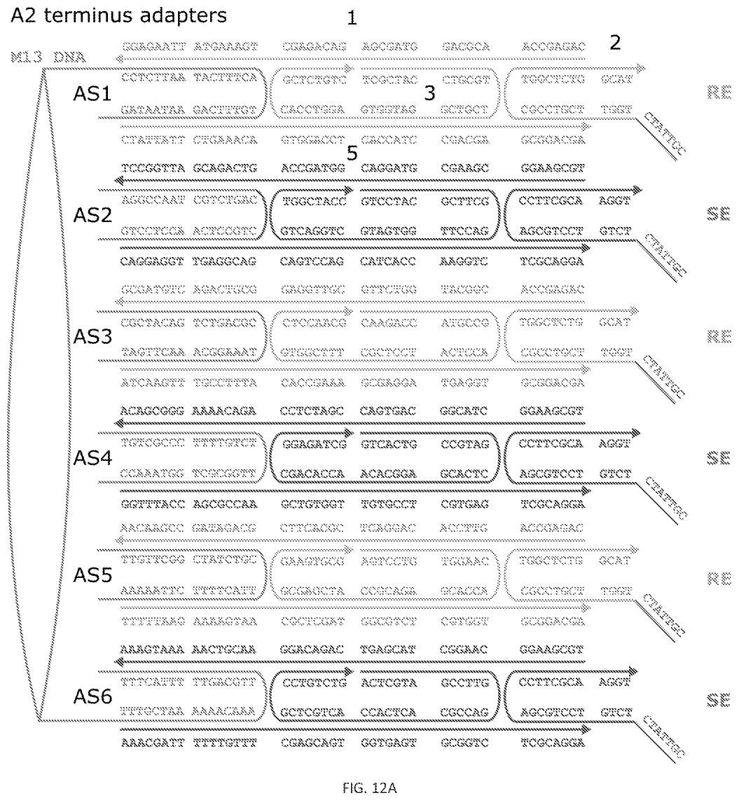

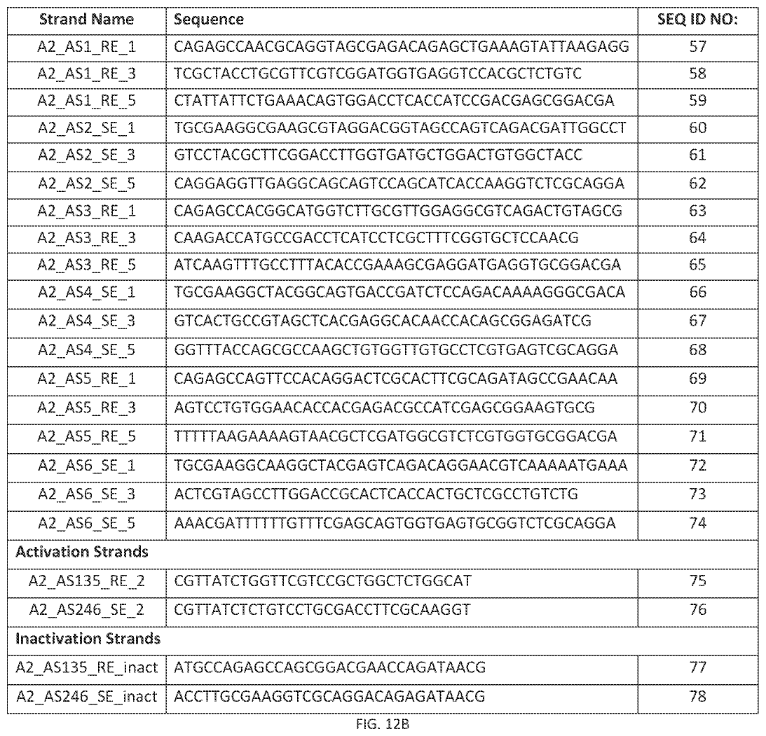

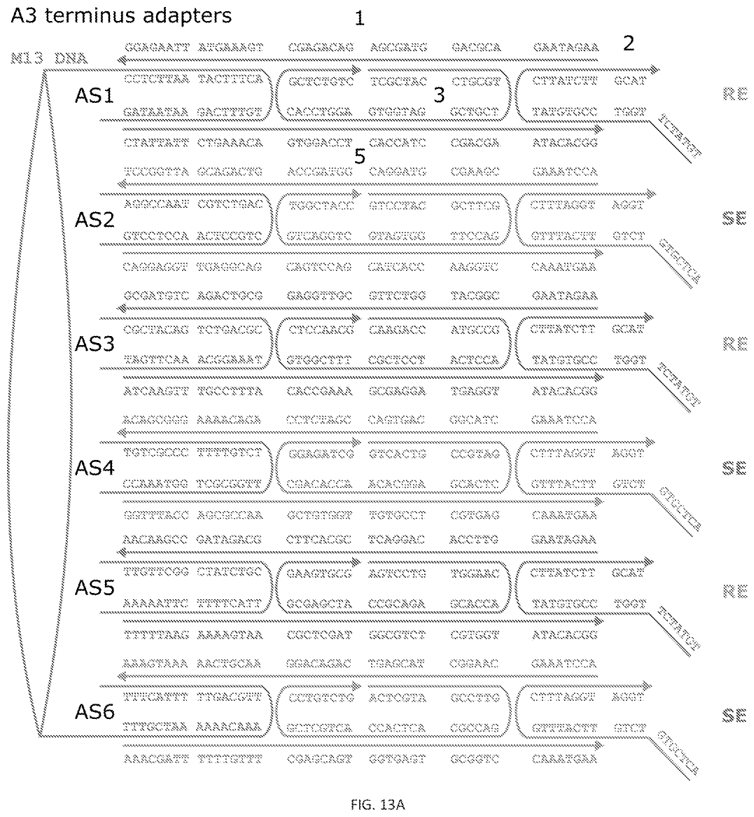

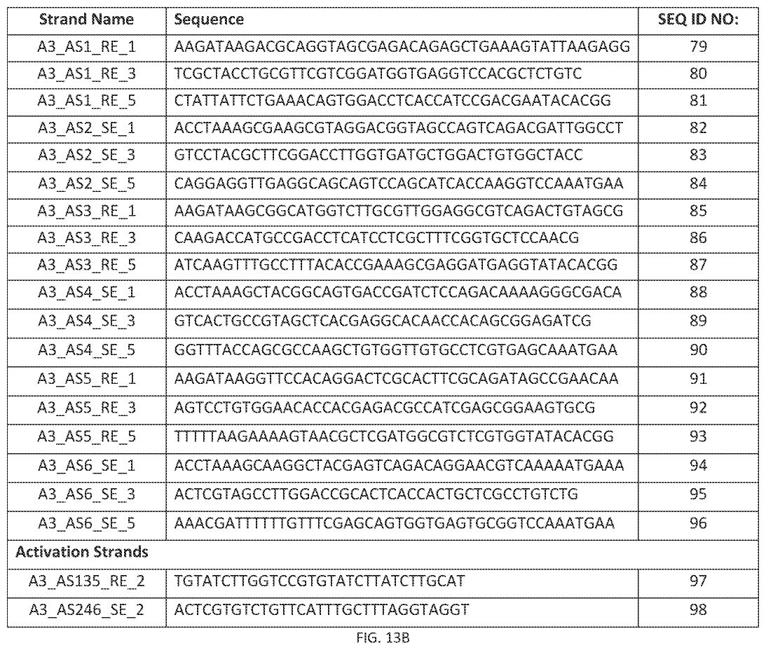

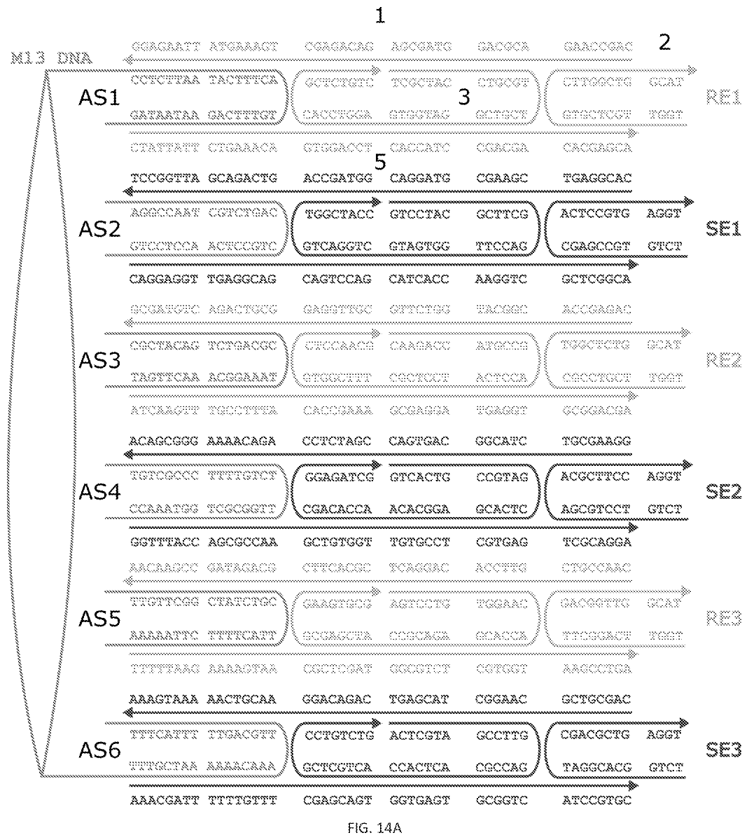

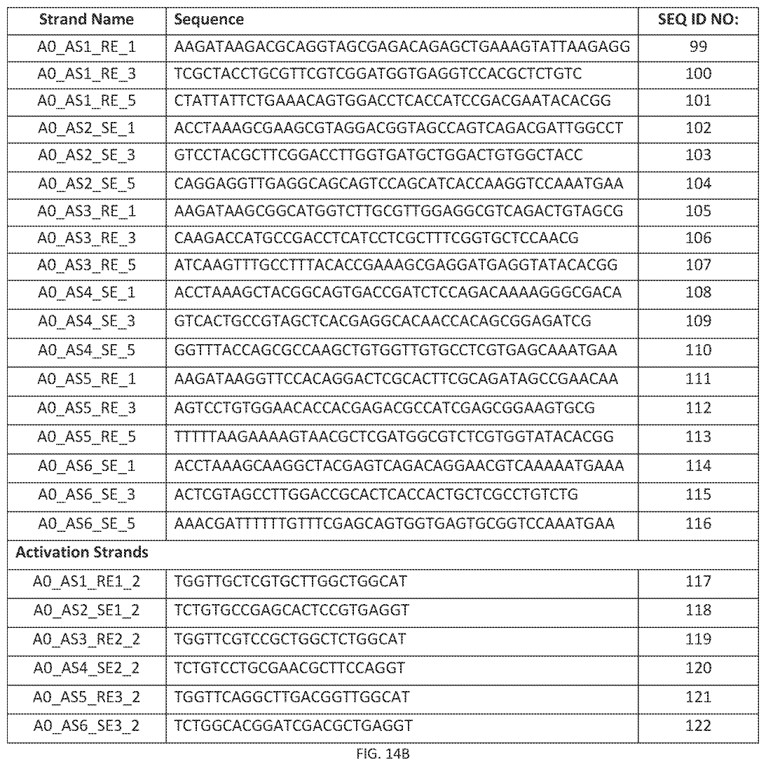

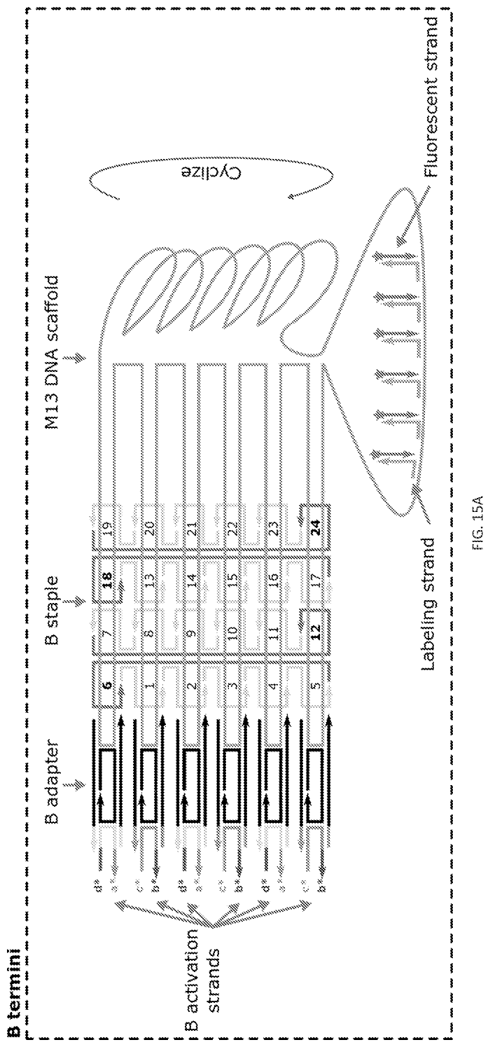

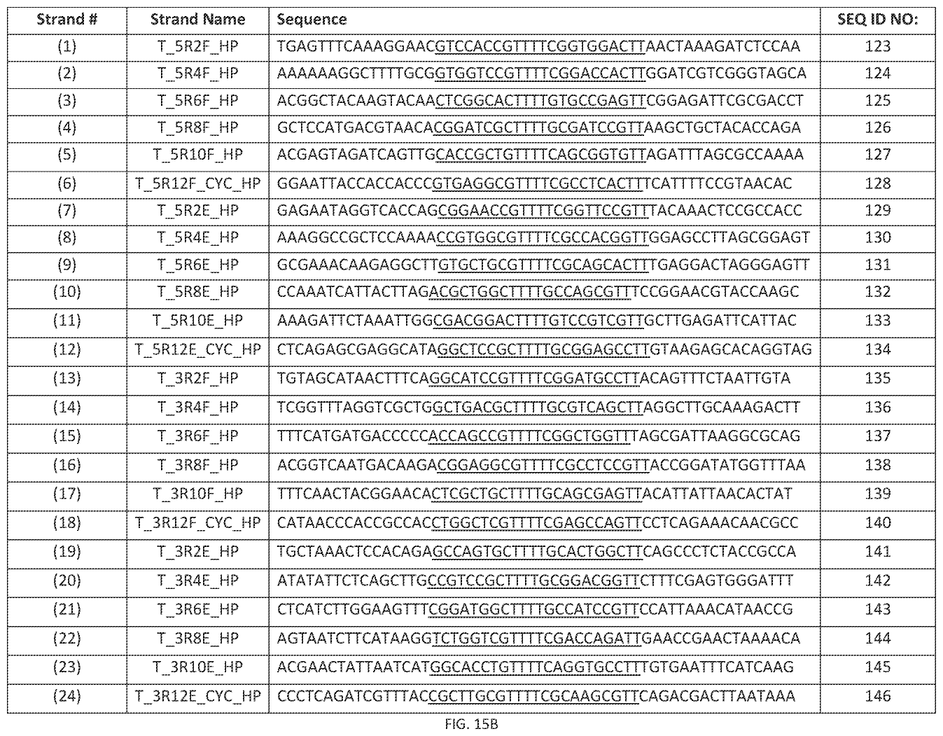

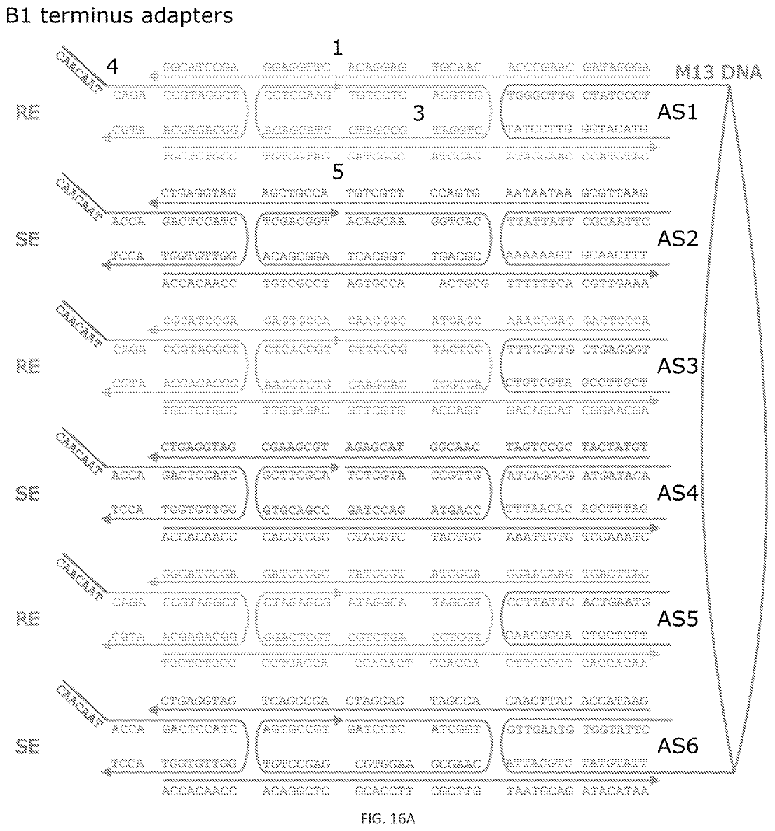

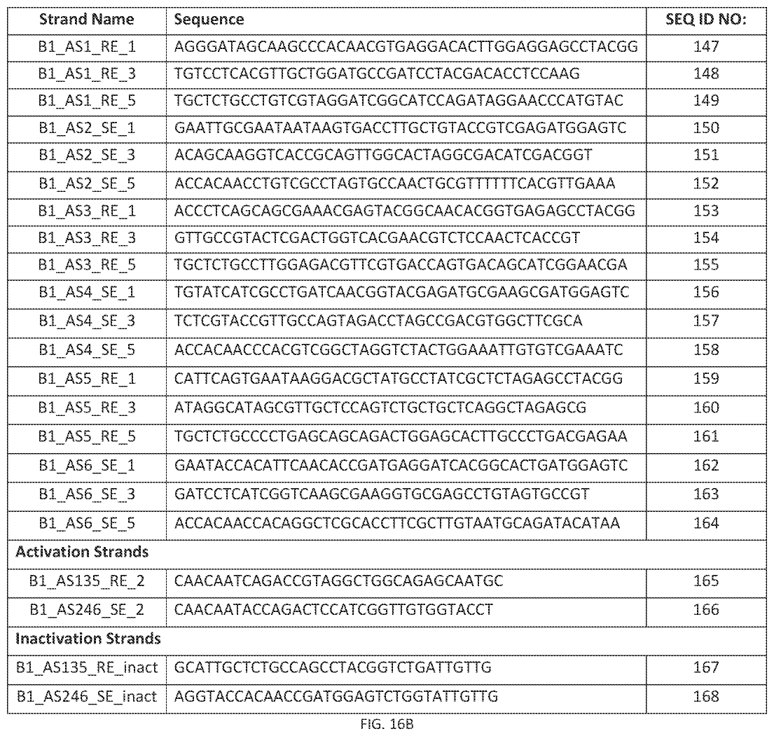

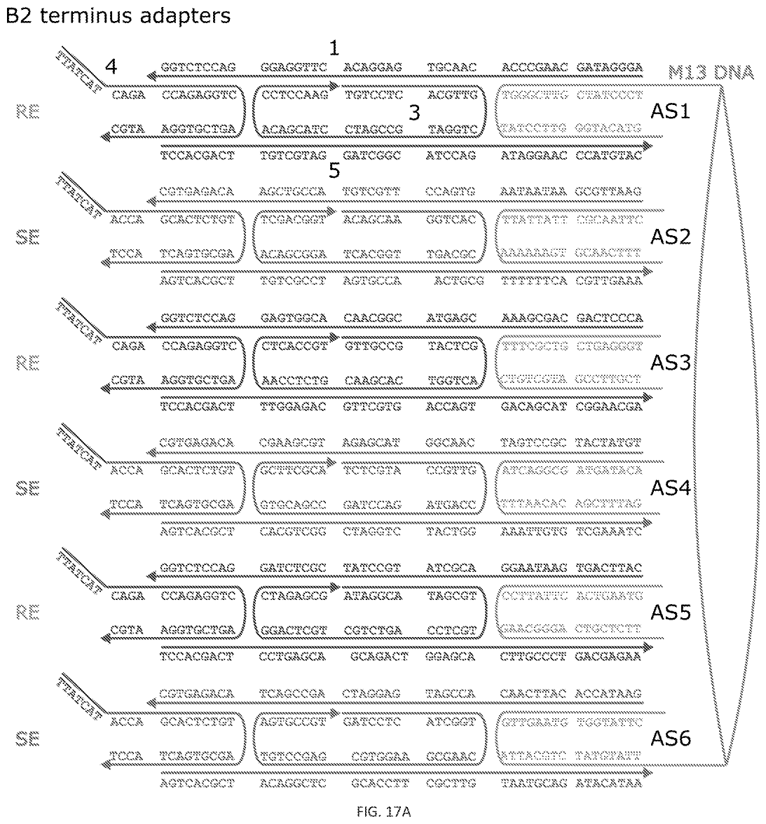

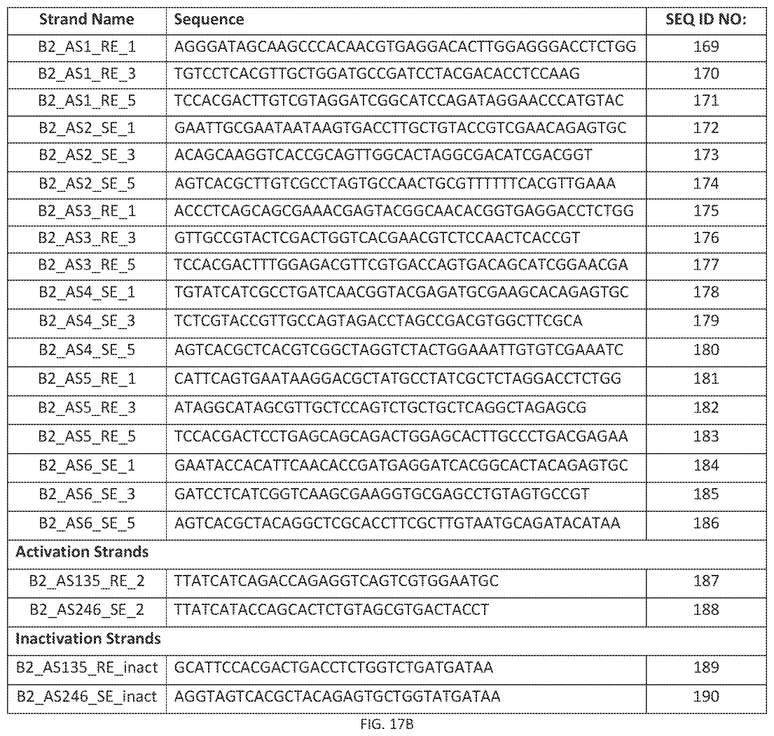

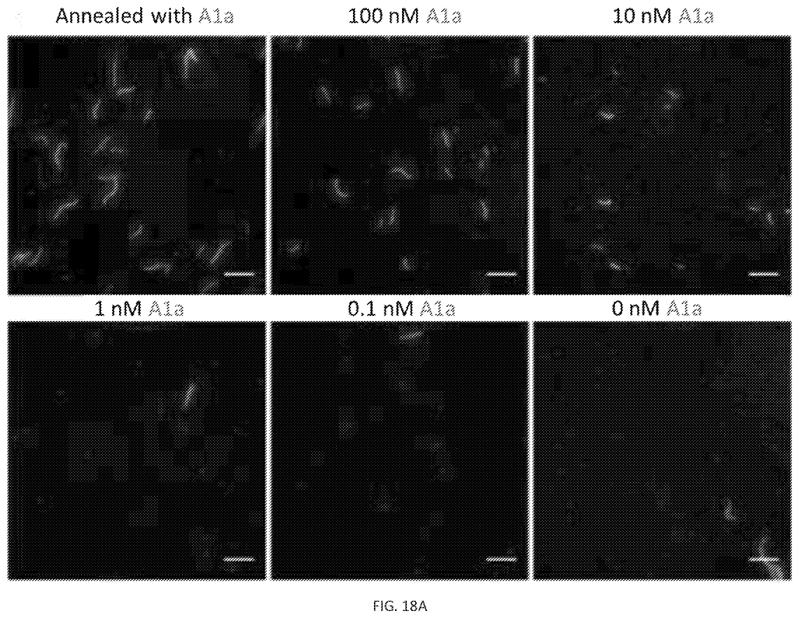

DNA components All oligonucleotides used in this study were synthesized by Integrated DNA Technologies (IDT). The sequences of the DNA monomers are in FIG. 8. M13mp18 DNA (7.240 bases) was purchased from Bayou Biolabs. The sequences of the staple strands, adapter strands, and activation/inactivation strands of the DNA origami termini are in FIGS. 10-17. The labeling strands for the DNA origami termini have the same structure and sequence as those used previously (Agrawal, D. K., et al., ACS Nano 2017, 11 (10), 9770-9779, incorporated herein by reference in its entirety).

Preparation of DNA monomer mixtures and DNA origami termnini DNA origami termini were annealed in an Eppendorf Mastercycler in 40 mM Tris-Acetate, 1 mM EDTA buffer supplemented with 12.5 mM magnesium acetate (TAEM). A DNA origami terminus is composed of a scaffold strand (M13mp18 DNA), 24 staple strands, and 18 adapter strands. Termini were fluorescently labeled using a mixture of labeling strands that bind to unfolded M13 DNA and provide a docking site for a fluorescently labeled strand (100 labeling sites per terminus) (FIG. 9). Inactive DNA origami termini were annealed in TAEM buffer with 5 nM M13 DNA, 500 nM of each staple strand, 100 nM of each adapter strand, 10 nM of each labeling strand, and 1000 nM of the fluorescently labeled strand. Termini annealed in an active state also included each activation strand at 600 nM. Biotinylated-BSA at a final concentration of 0.05 mg/mL (Sigma-Aldrich) was also included to prevent termini from sticking to the walls of the annealing tubes. Annealing was conducted as follows: samples were incubated at 90° C. for 5 min, cooled from 90° C. to 45° C. at 1° C./min, held at 45° C. for 1 hour, and then cooled from 45° C. to 20° C. at 0.1° C./min. After annealing, termini were purified with a centrifugal filter (100 kDa Amico Ultra-0.5 mL) to remove excess staple, adapter, and labeling strands. For purification, 50 μL of the annealed terminus mixture and 350 μL of TAEM buffer were added to the filter and centrifuged at 2000 RCF for 4 min. The samples were washed three more times by adding 200 μL of TAEM buffer to the remain solution and repeating centrifugation. The final sample was eluted by inverting the filter into a fresh tube and centrifuging briefly. Purified termini were stored at room temperature until used. Typically, termini were annealed the day before they were used.

Terminus activation and inactivation experiments To test terminus activation and inactivation, DNA monomer strands were freshly annealed in an Eppendorf Mastercycler in TAEM. Monomer mixes contained 90 nM of the RE and SE monomer sticky end strands (strands 2 and 4 in FIG. 8A) and 45 nM the other RE and SE strands (strands 1, 3, and 5 in FIG. 8A). Biotinylated-BSA at a final concentration of 0.05 mg/mL (Sigma-Aldrich) was also included to prevent monomers from sticking to the walls of the annealing tubes.²⁶ For each experimental sample, enough monomer mix was prepared to split into aliquots for each experimental timepoint prior to annealing. To anneal the monomers, samples were initially held at 90° C. for 5 min. cooled to 20° C. at −1° C./min, and subsequently held at 20° C. for the duration of the experiment. As soon as the samples reached 20° C. the appropriate DNA origami termini were added to all the aliquots to begin the experiment. DNA origami termini were typically added to a final concentration of 5-10 pM. After an initial incubation period followed by fluorescence imaging, the appropriate DNA activation or inactivation strands were added to all of the aliquots and the samples were imaged again after another incubation period. Unless otherwise noted, activation strands were added to a final concentration of 50 nM and inactivation strands were added to a final concentration of 100 nM. The lengths of the incubation periods varied between experiments, as noted.

Fluorescence imaging and analvsis Fluorescence imaging was conducted on an inverted microscope (Olympus IX71) using a 60×/1.45 NA oil immersion objective with 1.6× magnification. Images were captured on a cooled CCD camera (iXon3, Andor). For fluorescence imaging, 1 μL was taken from the appropriate timepoint aliquot and diluted 6-fold in TAEM containing 100 nM pre-annealed, non-fluorescent RE monomers without sticky end strands to reduce background fluorescence from unincorporated monomers. After dilution, 6 μL prepared sample was added to an 18 mm by 18 mm glass coverslip (VWR) that was then inverted onto a glass slide (VWR). Images were then captured at 3 to 5 randomly selected locations.

All fluorescence images were processed and analyzed using custom MATLAB scripts. Three to five images were typically processed and analyzed for a specific sample at a given timepoint.

Detecting objects A fluorescence micrograph of DNA nanotubes and a corresponding fluorescence micrograph of DNA origami termini were imported simultaneously for analysis. Canny edge detection6 was used to detect the edges of objects in both the DNA nanotube image and the DNA origami termini image and produce binary images of the object edges. The detected objects were then filled in with pixels using MATLAB's bwmorph( ) function. No further processing was done to the DNA origami termini image. For the DNA nanotube image, morphological operations were applied with MATLAB's bwnorph( ) function to skeletonize all the detected objects to be 1 pixel in width. This processed image was used for the quantification of the fraction of termini with nanotubes

Quantification of fraction of termini with nanotube—singly fluorophore To quantify the fraction of termini with nanotubes, the locations of the endpoints of each object in the qFTwN image were determined. A radius (typically 2 to 4 pixels) around each of these endpoint locations was searched in the processed DNA origami termini image and if a terminus was found in the search radius, this terminus was counted as having a nanotube attached to it. The fraction of termini with nanotubes was then calculated as the total number of identified termini that had a nanotube attached to them divided by the total number of termini in all the images processed for a given set of conditions (experiment and timepoint). The total number of termini in each image was determined by counting all the individual objects in the processed DNA origami termini image. Error bars for the fraction of termini with nanotubes represent the 95% confidence intervals of proportions for large samples (Eq. 1).

CI=±1.96\sqrt{p(1−p)/n} (1)

Quantification of fraction of nanotubes with termini—single fluorophore

To quantify the fraction of nanotubes with termini, the objects in the qFTwN nanotube image that extended past the boundary of the image were removed as it is not possible to determine what types of termini (if any) are present at both the ends of a nanotube that extends past the image boundary. We will term this processed nanotube image with the nanotubes that extended past the boundaries removed qFNwT below. To determine the fraction of nanotubes with termini, the locations of the endpoints of each nanostructure in the qFNwT image were determined. A radius (typically 2 to 4 pixels) around each of these endpoint locations was searched in the processed DNA origami terminus image and if a terminus was found in the search radius, a nanotube with a terminus was counted. The fraction of nanotubes was then calculated as the total number of nanotube endpoints that had a specific terminus attached to them over the total number of nanotubes across all the images processed for a given sample at a specific timepoint. Since some nanotubes cross over in the images and result in branched objects with more than two endpoints in the processed images, the total number of nanotubes in an image was calculated as:

$$ceil ( \frac{\# of endpoints}{2} ) \qquad ( 2 )$$

where ceil(x) rounds to the lowest integer greater than or equal to x. So, an object with two endpoints would be counted as a single nanotube, an object with three or four endpoints would be counted as two nanotubes, an object with five or six endpoints would be counted as three nanotubes, etc. Error bars for the fraction of termini with nanotubes represent the 95% confidence intervals of proportions (Eq. 1).

For samples with termini that were labeled with two different fluorophores, adjustments to the analysis were made to account for termini labeled with two fluorophores. First, termini labeled with two fluorophores were identified by identifying image locations where the pixels of an object in the processed atto488 binary micrograph overlapped more than 50% of the pixels of an object in the processed atto647 binary micrograph. Second, the total number of termini labeled with only a single fluorophore in each image were then quantified by counting all the individual objects in the processed termini binary micrograph of a single fluorophore and subtracting from that quantity the total number of identified termini labeled with two fluorophores. Once the number and location of all the different termini we located the fraction of nanotubes with termini and termini with nanotubes could be calculated as described in above.

In experiments where both an A terminus and a B terminus were activated, the yield of nanotubes attached to both an A and B terminus was calculated. We termed such nanotubes with termini on both ends, doubly terminated nanotubes. The yield of forming doubly terminated nanotubes was determined by analyzing the qFNwT processed nanotube images. The yield was then calculated as the total number of nanotubes attached to both the termini of interest divided by the total number of nanotubes in all the images processed for a given sample at a specific timepoint (Eq. 3).

$$\frac{\# of nanotubes attached to {T}_{1} and {T}_{2}}{total \# of nanotubes} \qquad ( 3 )$$

where T1 is the terminus that was activated first and T2 is the terminus that was activated second in the experiments.

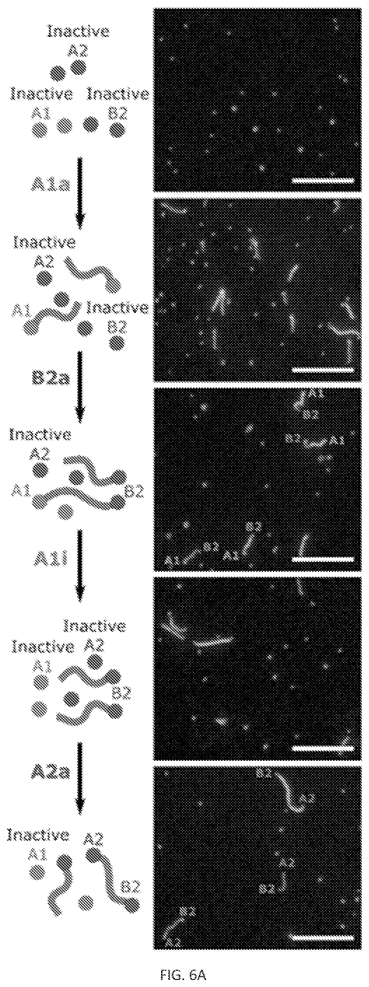

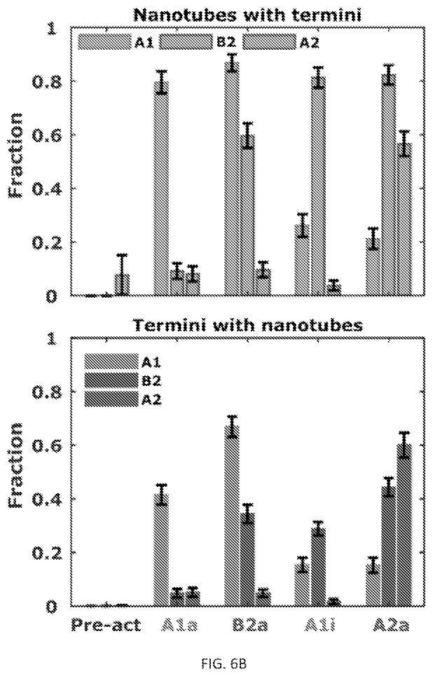

Yields were also calculated by dividing by the number of nanotubes attached to either T1 or T2 (Eqs. 4 and 5). For example, for the results in FIG. 5B the yield with respect to A 1 nanotubes is the number of nanotubes to both and A1 and B1 terminus divided by all the nanotubes attached to an A1 terminus.

$$\frac{\# of nanotubes attached to {T}_{1} and {T}_{2}}{\# of nanotubes attached to {T}_{1}} \qquad ( 4 )$$

$$\frac{\# of nanotubes attached to {T}_{1} and {T}_{2}}{\# of nanotubes attached to {T}_{2}} \qquad ( 5 )$$

### Example 1

### Activation and Inactivation of DNA Origami Termini

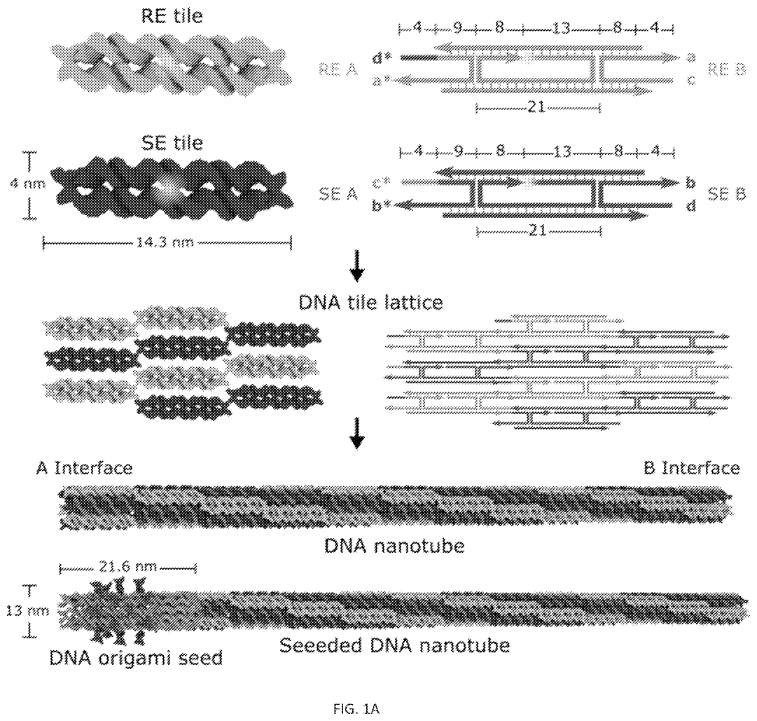

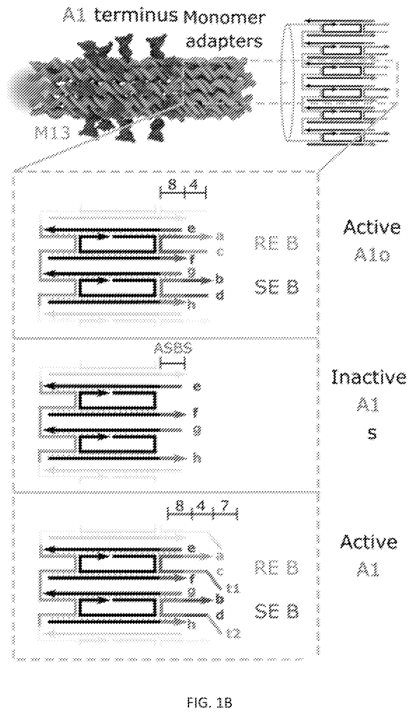

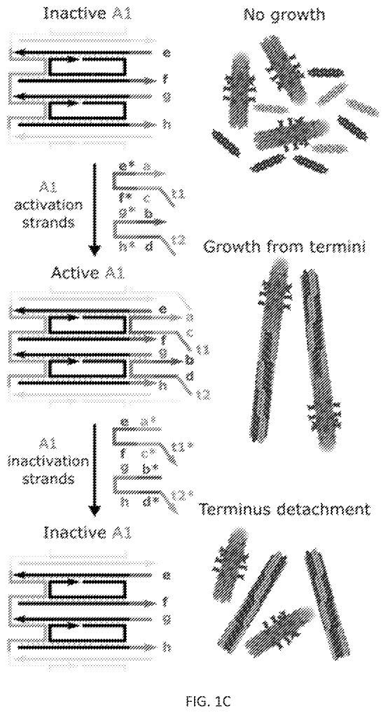

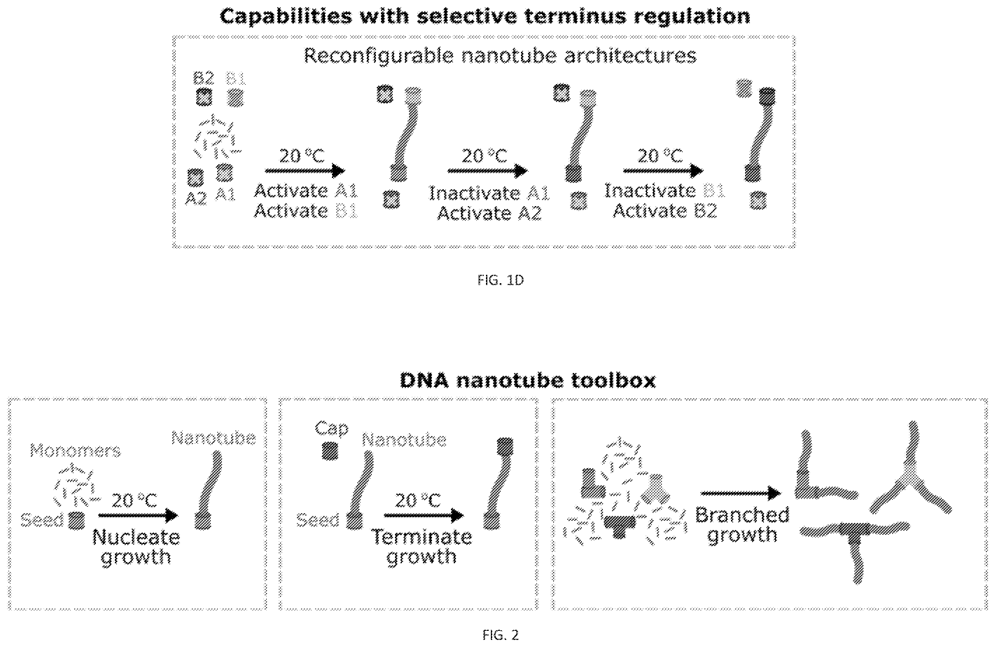

A mechanism was designed to use unique DNA sequences to specifically activate and inactivate a DNA origami structure that has been used as both a seed for nucleating nanotube growth and a cap for terminating growth. The structure was termed a DNA origami terminus, since it can serve as either a nanotube seed or cap and is attached to the end of a nanotube (FIG. 2A). A DNA origami terminus interacts with DNA monomers or nanotubes via monomer adapter structures. There are six adapter structures around the interface of a terminus that each present monomer sticky end sequences; together these structures mimic the structure of a nanotube growth facet. There are three adapter structures that present RE sticky ends and three adapter structures that present SE sticky ends (FIG. 1B; top panel). For the terminus shown in FIG. 1B, an A1 terminus, the adapter structures present the sticky end sequences that allow them to bind to the A interface of the monomers to nucleate growth or join with the A interface of DNA nanotubes to terminate growth.

A method to control terminus activity was created in which inactive termini are missing the strands that present the sticky end sequences on all six of the adapter structures, meaning that the terminus cannot interact with monomers or nanotubes (FIG. 1B; middle panel). This inactive terminus is activated by adding the adapter sticky end strands (called the activation strands) that specifically bind to the interface of the inactive terminus (FIG. 1C). The activation strand binding sites (ASBS in FIG. 1B; middle panel) on the adapter structures that bind the RE activation strands were designed to each have the same sequence and the ASBS of the SE activation strands likewise have the same sequences. In this design, only two unique sequences are required to activate a terminus.

Inactivating a terminus that is bound to the end of a DNA nanotube in the design involves removing the adapter sticky end strands from the bound terminus. To facilitate these strands' removal, the activation strands were designed to include (FIG. 1C: bottom panel). These single-stranded domains facilitate a toehold-mediated DNA strand displacement reaction where strands complementary to the activation strands bind to the activation strands on a terminus and remove them. This removal inactivates the terminus, and if the terminus is bound to a DNA nanotube, removes the terminus from the nanotube end (FIG. 1C).

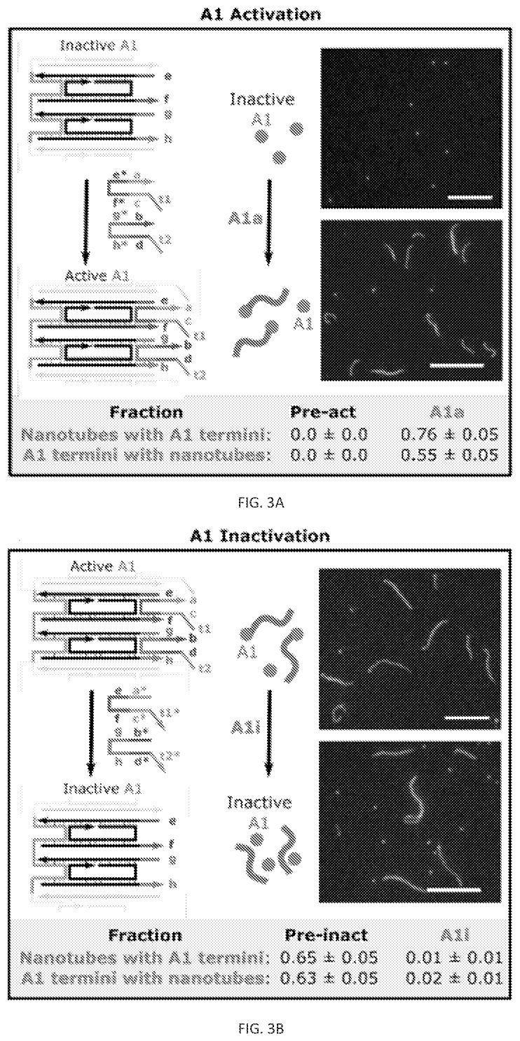

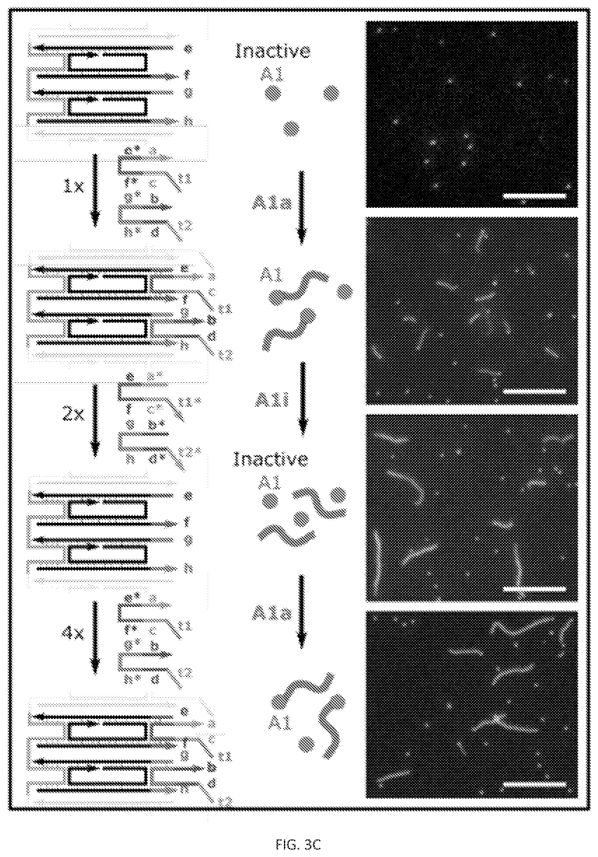

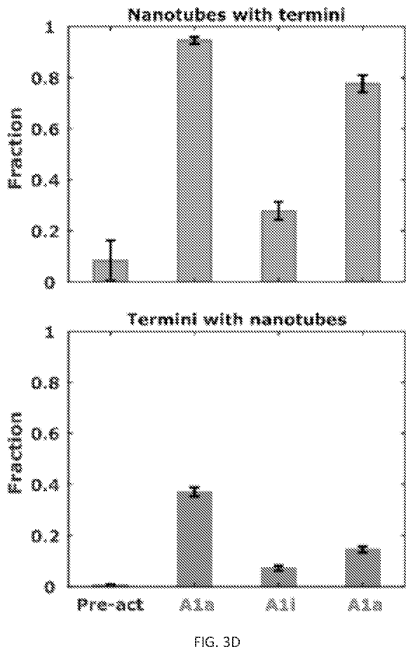

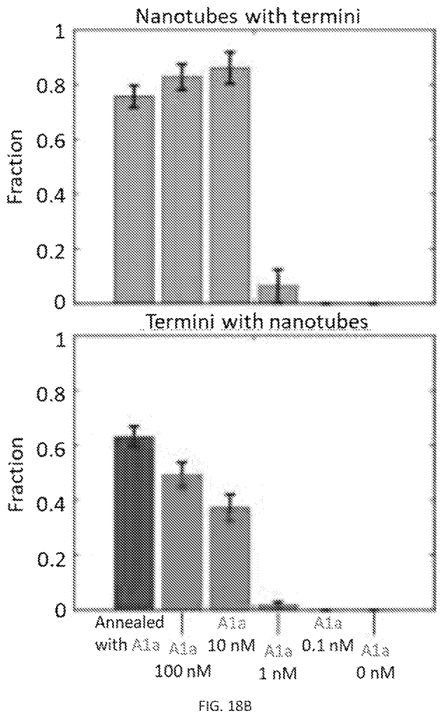

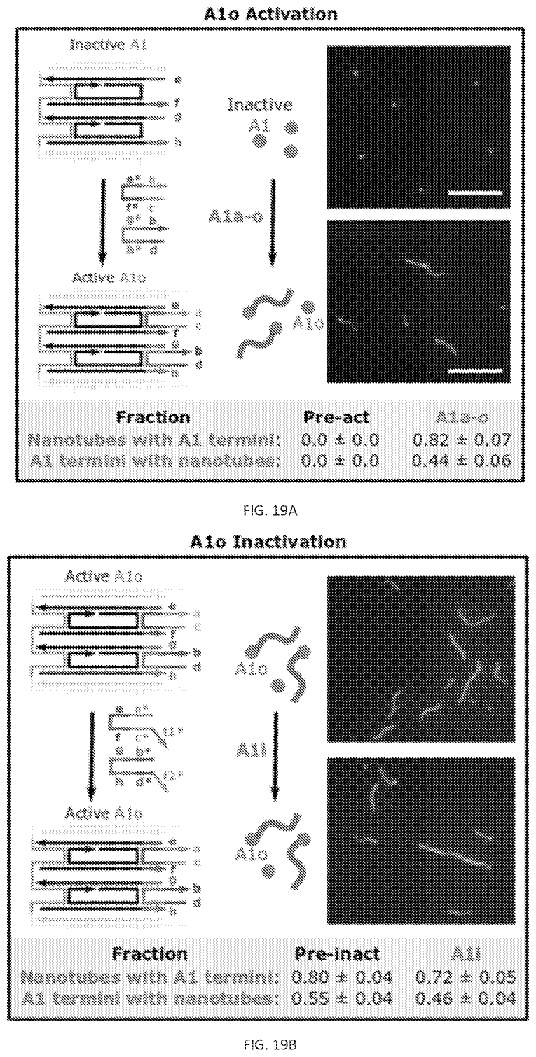

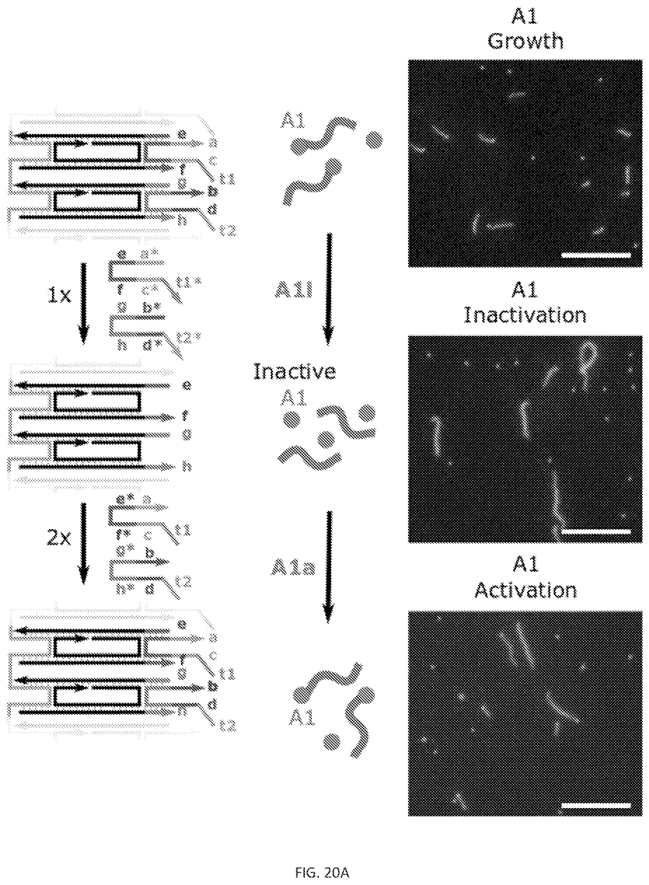

Inactive A1 termini were incubated with 45 nM DNA monomers, a monomer concentration at which nanotubes could grow from termini but only rarely nucleated homogeneously. No termini nucleated nanotube growth (N=91) after 6 or 24 hours of incubation (FIGS. 3A and 18, respectively) and 5-fold fewer nanotubes were observed than when the same concentration of active termini was incubated with monomers (FIG. 18). Terminus activation was tested by adding the A 1 adapter sticky end strands (activation strands) to inactive A1 termini after a six-hour incubation with 45 nM monomers. Twenty-one hours after the addition of the activation strands, nearly 50% of the termini had nucleated nanotube growth and nearly 80% of nanotubes that had grown were attached to termini (FIG. 3A). Similar fractions of activated termini with nanotubes and of nanotubes attached to termini were observed when using activation strands with and without the 7-base single-stranded toehold domains (FIGS. 3A and 19A), indicating that the toeholds do not significantly affect the process of nanotube nucleation from an active terminus. Given that only 50-60% of termini annealed in an active form nucleated nanotubes (FIGS. 3B and 18), the 50% yield of active termini after the addition of the activation strands indicates that activation recovers over 80% of terminus activity. The relatively low yield of nanotube growth from termini may be the result of using unpurified adapter and activation strands, as a significant fraction have defective adapter structures (which raises the barrier to nucleation from these structures).

Termini were annealed with the Ala strands, incubated with 45 nM monomers to nucleate nanotube growth, and after 22 hours of growth, inactivation strands were added complementary to the A1a strands (A1i). Nearly all of the termini with the 5′ toehold domains detached from the nanotubes (<2% of termini still attached) upon addition of the inactivation strands (FIG. 3B). No detachment of termini whose activation strands lacked the single-stranded 5′ domains was observed (FIG. 19B). Termini can therefore be reliably inactivated following the designed pathway of toehold-mediated strand displacement the activation strands from the termini.

### Example 2

### Sequential Activation and Inactivation of Termini

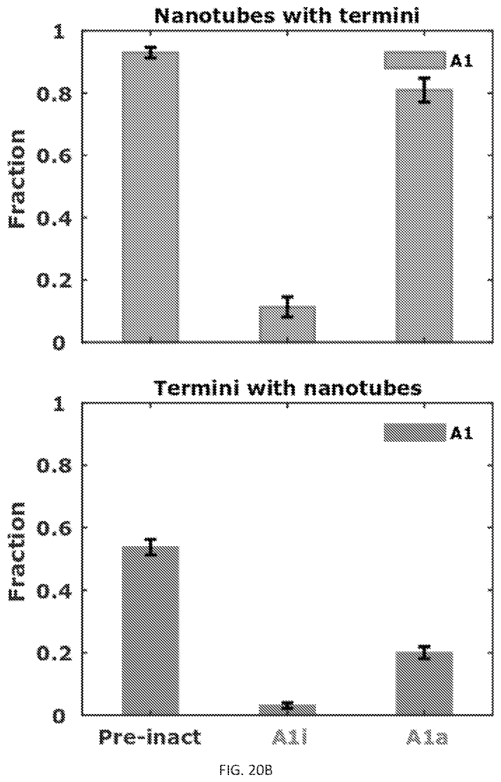

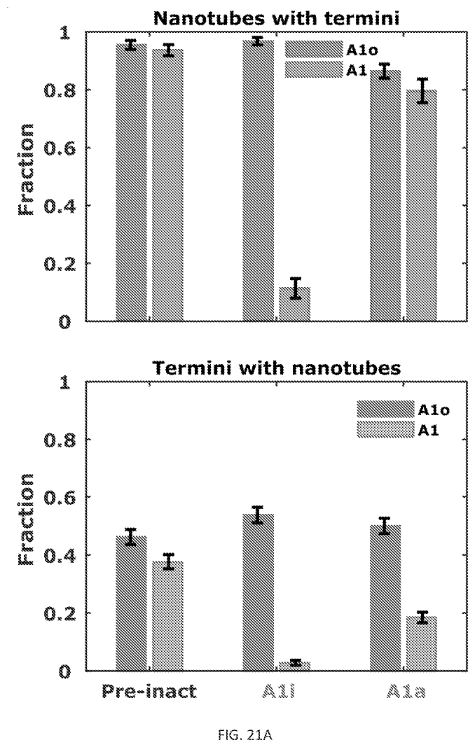

A reconfigurable nanotube architecture might be expected to take on many different morphologies over time, involving multiple steps of activation and inactivation of specific termini depending on the reconfiguration pathway. Inactivation followed by activation was tested by growing nanotubes from active A1 termini, adding inactivation strands (A1i) and after terminus inactivation, adding activation strands (A1a). 80% of the termini were removed from the nanotubes within 4 hours of the addition of the inactivation strands and over 80% of the nanotubes in solution were attached to termini after the reactivation step (FIG. 20). Inactive A1 termini could also be mixed with monomers, activated to grow nanotubes (resulting in >95% nanotubes with termini), then inactivated (leaving <30% nanotubes with termini), and finally reactivated (reattaching roughly 80% of nanotubes to the termini) (FIGS. 3C and 3D). To ensure that each activation or inactivation step removed all of the activation or inactivation strands from the previous step, the concentration of the strands added at each step was increased (FIG. 3C).

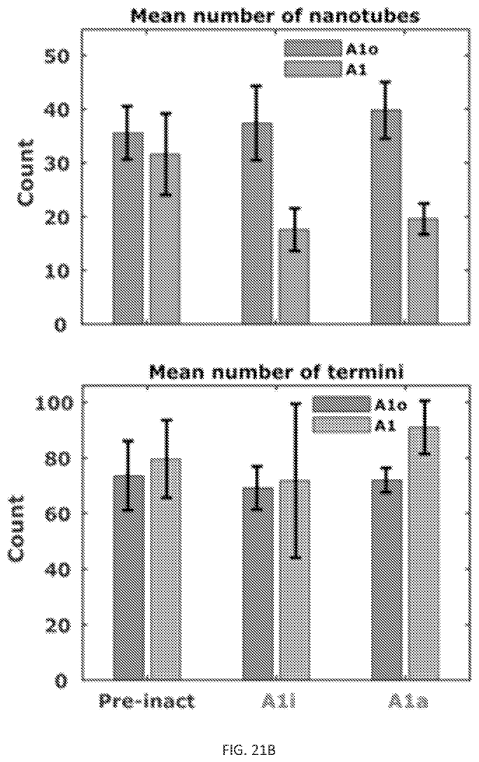

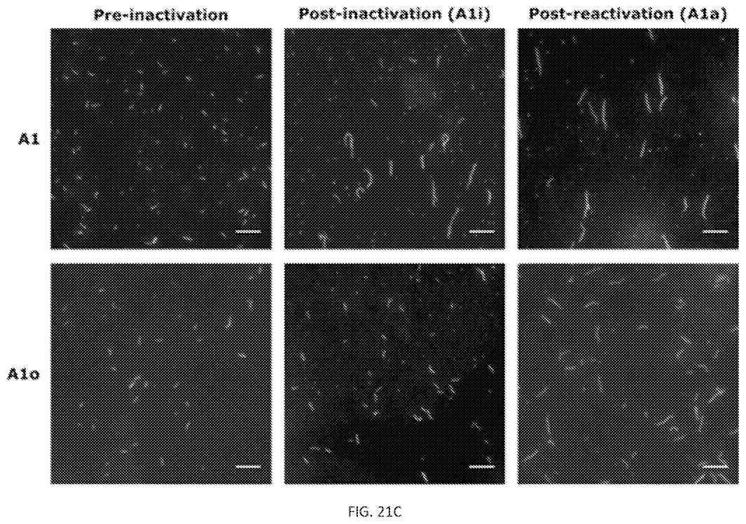

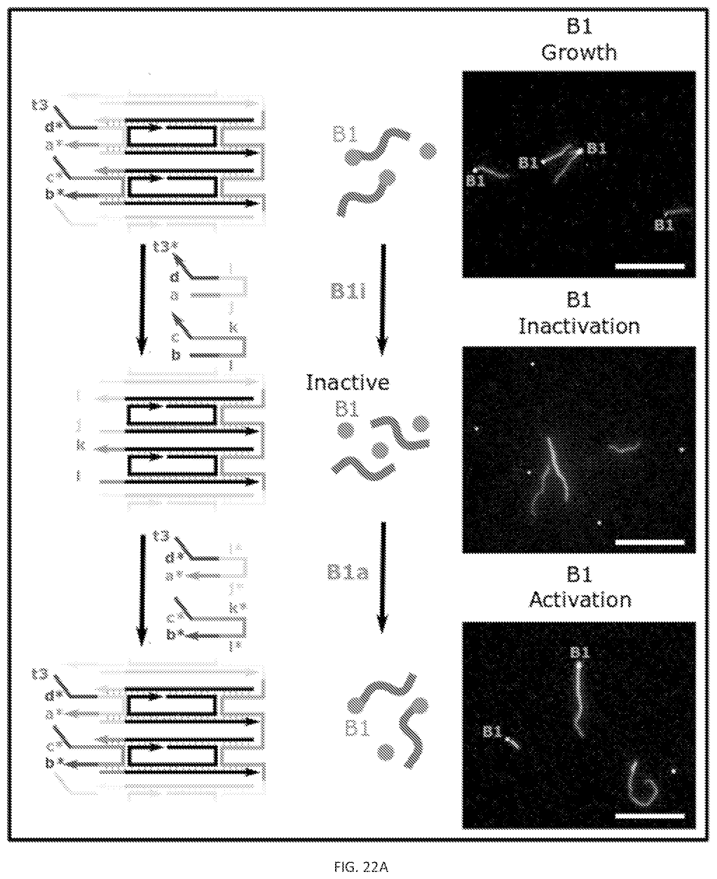

When measured as the fraction of nanotubes attached to termini, reactivation appears to proceed almost to completion. However, the fraction of termini with nanotubes on them is 30% lower after the second activation step than after the first (FIG. 3D). After terminus inactivation, when both the A and B interfaces of the nanotubes are free, end-to-end joining of nanotubes may reduce the number of nanotubes in a sample (e.g., the concentration of nanotube ends). Since the concentration of the termini does not change, end-to-end joining decreased the maximum yield of termini with attached nanotubes that could be achieved (FIG. 21).

### Example 3

### Orthogonal DNA Origami Termini that can be Selectively Activated and Inactivated

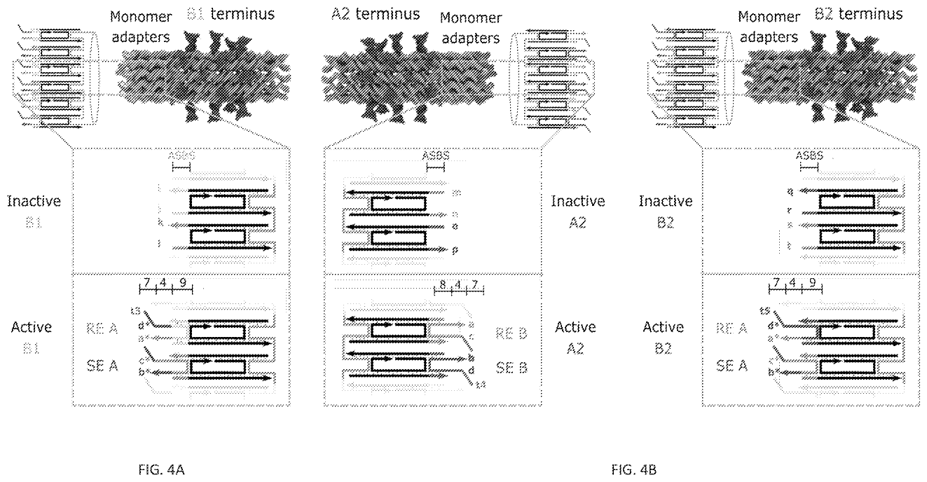

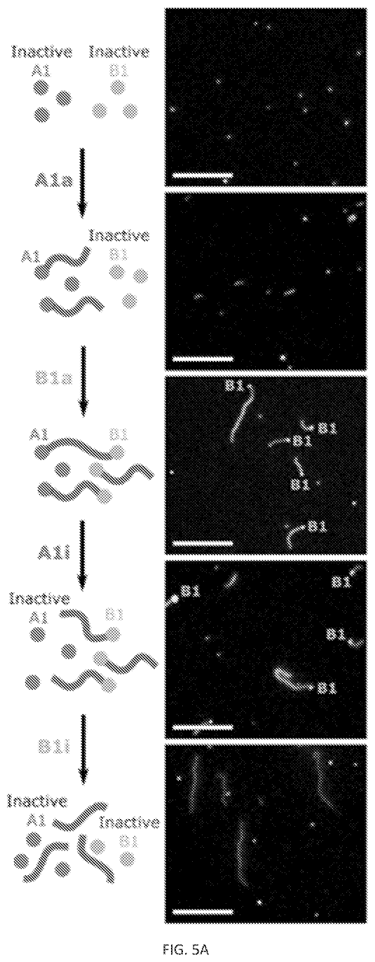

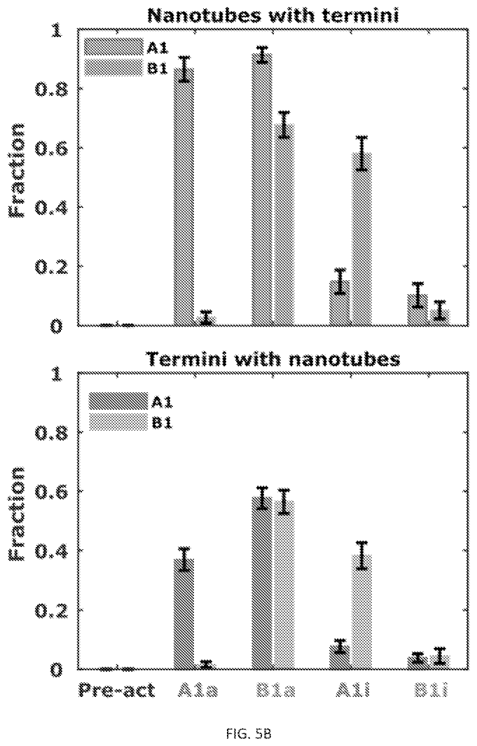

The ability to activate/inactivate specific DNA origami termini that bind to different nanotube binding interfaces could enable building and reconfiguration of more complex nanotube architectures (FIG. 1D). To demonstrate how multiple structures could be attached and detached from each of the two ends of a nanotube, a terminus that could bind to the B interface of the DNA monomers or nanotubes e.g., on the opposite side of the DNA nanotubes from where the A1 terminus binds (the B1 terminus in FIG. 4A) was designed. To prevent binding crosstalk during activation or inactivation of the A1 or B1 termini, the sequences for the activation strand binding sites (ASBS) on the adapter structures of the B1 terminus were designed to be distinct from those used for the ASBS of the A1 terminus (FIG. 4A). A second A terminus (A2) and a second B terminus (B2), each with their own unique ASBS sequences, were also designed along with their own activation and inactivation strands (FIG. 4B). After activation and inactivation of each of these individual termini in isolation (B1, A2, or B2), >75% of nanotubes were attached to termini after activation and <20% of nanotubes were attached to termini after inactivation. The two B termini had lower nucleation yields (fraction of termini with nanotubes) than the two A termini.

### Example 4

### Doubly Terminated Nanotube Architectures

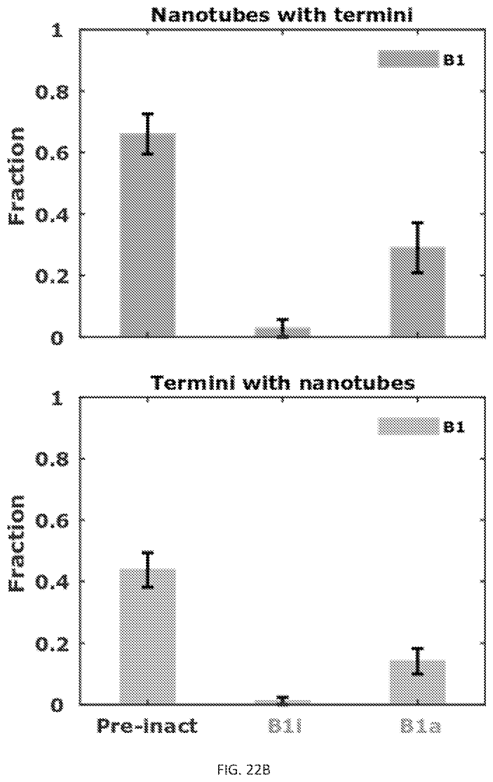

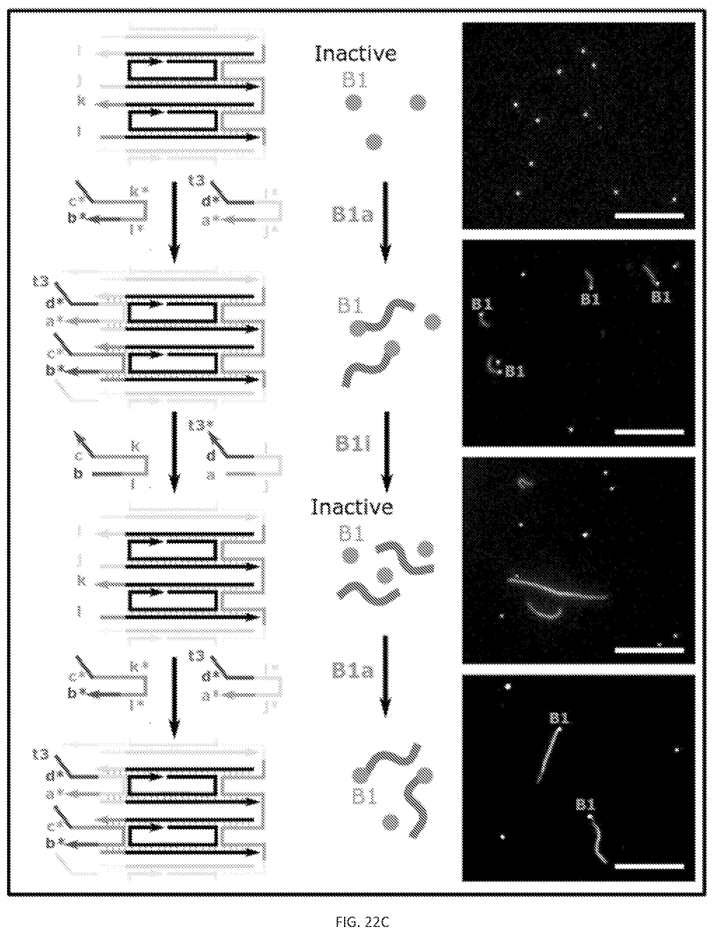

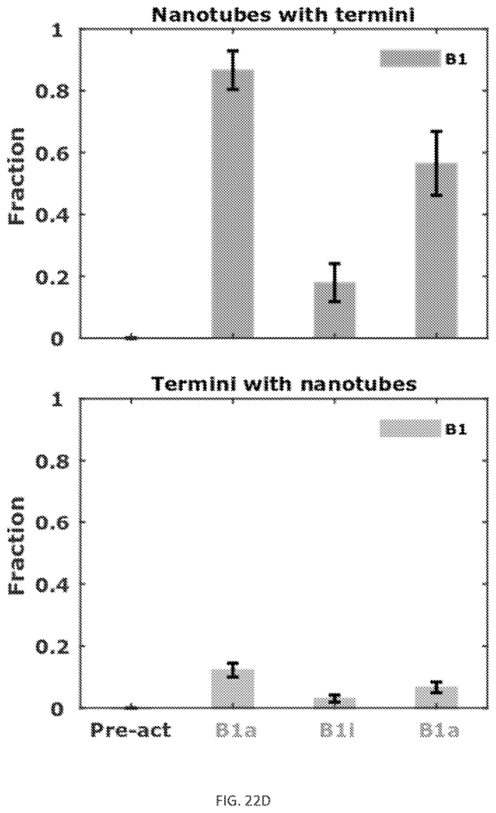

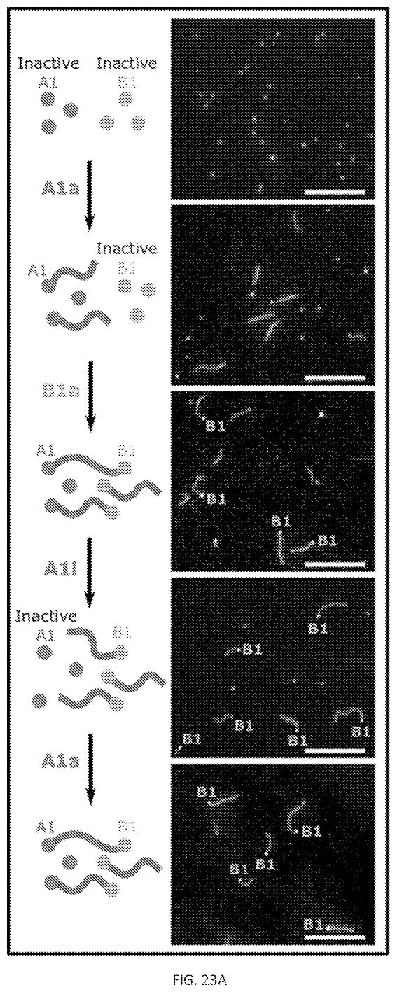

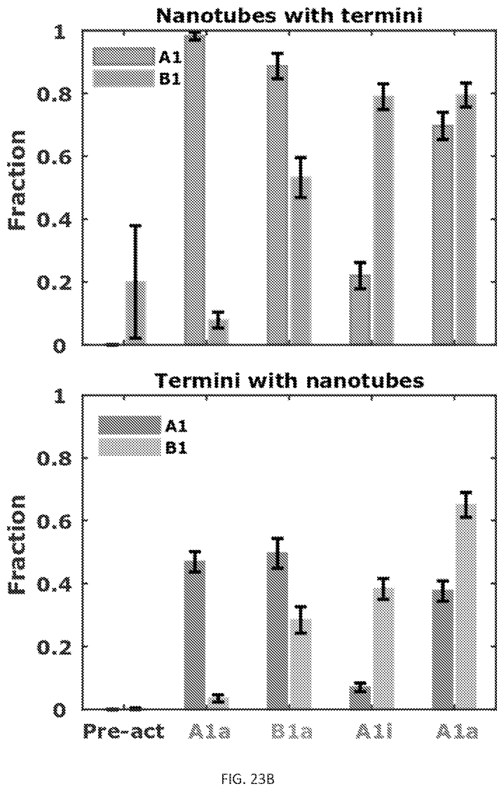

Inactive A1 and B1 termini were combined with monomers, then Ala strands were added to initiate growth from the A1 termini. After 5 hours, almost 90% of the nanotubes that grew were attached to A1 termini and fewer than 5% were attached to B1 termini, indicating selective A1 activation (FIG. 5). B1 activation strands were then added and after a 13-hour incubation, roughly 70% of the nanotubes were attached to B1 termini and 67% of nanotubes attached to B1 termini were also attached to A1 termini. Doubly terminated A1-B1 architectures can form by either an activated B1 terminus binding to a nanotube with an A1 terminus attached at its opposite end or an activated B1 terminus nucleating growth and then joining end-to-end with a nanotube attached to an A1 terminus. Given that when B1 termini are mixed with monomers, the activation of the B1 terminus resulted in less than 20% nucleation yield (FIG. 22), the majority A1-B1 terminated architectures likely formed because B1 termini bound to the ends of existing nanotubes. While 67% of the nanotubes attached to B1 termini were incorporated into A1-B1 terminated architectures, only 50% of nanotubes attached to A1 termini were also attached to B1 termini. If each termini's activation efficiency is 50-60%, it would be expected that only half of the nanotubes attached to A1 termini would also become attached to B1 termini. Sequential addition of A 1 and then B1 inactivation strands successively removed termini from both nanotube ends, leaving no nanotubes with both termini still attached (N=59). These results demonstrated that the A1 and B1 activation/inactivation strands can selectively control the activity of their DNA origami targets and that doubly terminated nanotube architectures can be selectively assembled and disassembled. An A1-B1 doubly terminated structure was also selectively assembled and then the A1 terminus was successively removed and reattached with similar yields (FIG. 23) to the results in FIG. 5, demonstrating doubly terminated structures can be dynamically formed, disassembled, and reformed.

Doubly terminated nanotube architectures can be reconfigured via selective terminus activation and inactivation As doubly terminated structures can link two termini bound to surfaces or other objects, switching which termini are attached could be used to change the surfaces or objects that a nanotube links. Inactive A1, B2, and A2 termini were combined with monomers. Then the A1 activation strands (A1a) were added to initiate growth from the A 1 termini. After 19 hours, 80% of the nanotubes that grew were attached to A1 termini and fewer than 15% were attached to either an A2 or B2 terminus (FIG. 6), demonstrating the specificity of A1 activation. B2 activation strands (B2a) were then added to form A1-B2 terminated nanotube architectures. After 6 hours, consistent with the results presented in FIG. 5, the majority (85.3%) of nanotubes attached to a B2 terminus were also attached to an A1 terminus. Additionally, the percentage of nanotubes attached to A2 termini still remained lower than 15% (FIG. 6), demonstrating specific B2 activation. To reconfigure the A1-B2 terminated structure to produce an A2-B2 terminated structure, the A1 inactivation strands (Ali) which detached the A1 termini from nanotubes, were next added reducing the percentage of nanotubes A1-B2 doubly terminated architecture to under 20% in 3.5 hours. Finally, the A2 activation strands (A2a) were added to form A2-B2 terminated nanotube architectures. After 24 hours, 75% of the nanotubes attached to A2 termini were also attached to a B2 termini, and thus were parts of A2-B2 terminated structures. These results demonstrate that nanotube architectures can be reconfigured by selectively regulating the activity of a library of DNA origami termini in a stepwise fashion.

### Example 5

### Terminus Activation for Sensitive Nucleic Acid Sequence Detection

The above results demonstrated that terminus activation and inactivation can be used to sequentially build and reorganize DNA nanotube architectures. Reorganizing an architecture by the activation and inactivation of termini is an efficient means of achieving large scale structural change or the initiation of a nanotube assembly process in response to only a small concentration of an input signal because there are typically over 1000-fold more monomers (present in our experiments at 45 nM) than termini (present in our experiments at approximately 3 pM). In principle, because only a few strands of DNA are required to activate (or inactivate) a terminus, and termini are present at picomolar concentrations, assembly of a new nanotube could occur in response to picomolar quantities of the input.

Initiation of DNA nanotube self-assembly in response to specific chemical signals (both nucleic acids and proteins) has previously been explored and proposed as a potential tool for point-of-care diagnostics. In these previous demonstrations, input signals converted inactive monomers into active monomers that then spontaneously self-assembled into nanotubes for detection. However, these methods and systems required input signal concentrations on the order of 100 nM to activate enough monomers for growth. The termini activation scheme herein could be much more sensitive as only 1-5 pM of termini may be needed to activate to initiate seeded growth. In addition, the system and method herein may detect short single-stranded sequences in situ at room temperature by using these strands to complete a mostly folded, but inactive nucleation site.

To investigate the sensitivity of terminus activation in response to sequence inputs, 0.1, 1, 10, and 100 nM of the activation strands were added to respective solutions of 3 pM inactive A1 termini and 45 nM nanotube monomers. In principle, activation should complete when just 9-10 pM of these strands are added. However, surprisingly, fewer than 2% of termini were activated when 1 nM of the activation strands were added (almost a 100-fold excess of activation strands with respect to their binding sites) and no detectable nucleation was observed with 0.1 nM of activation strands (FIG. 7A).

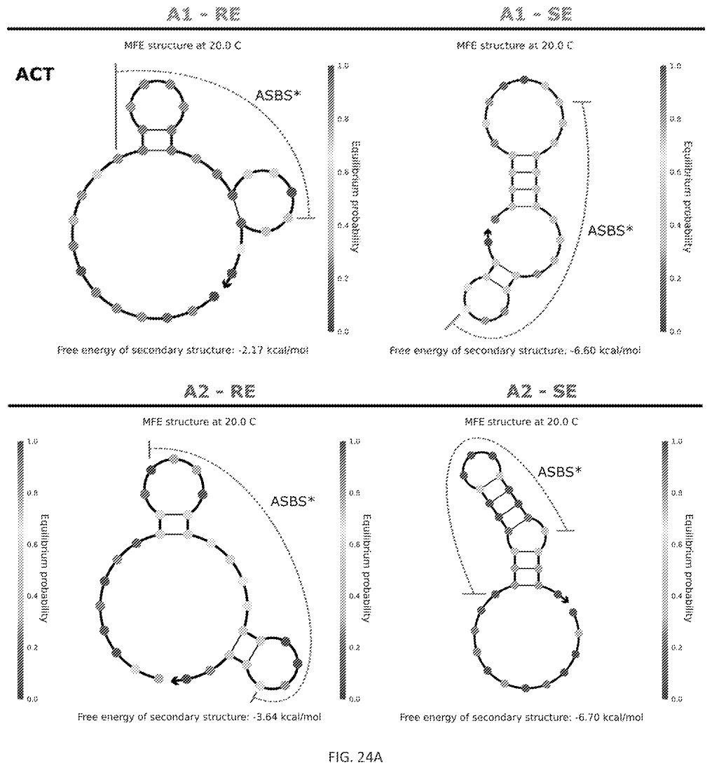

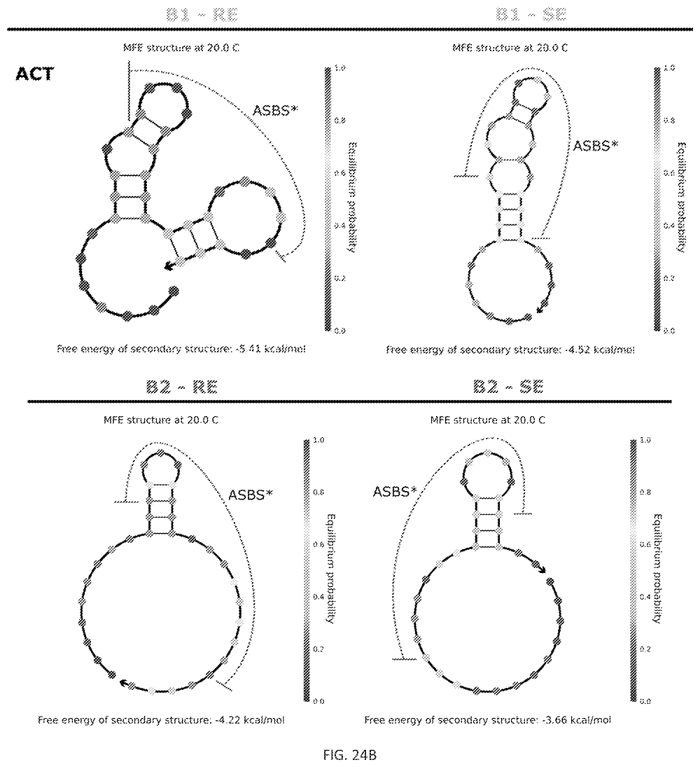

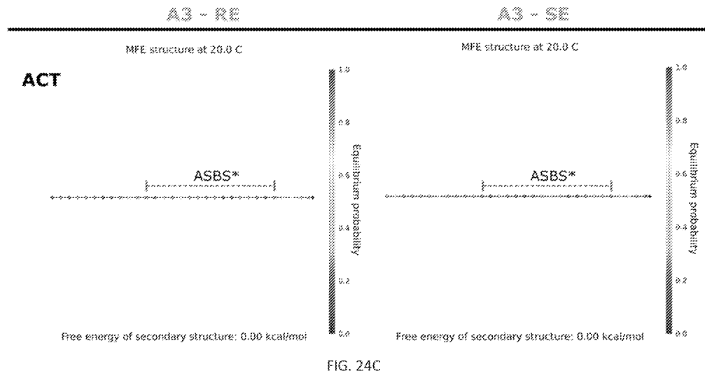

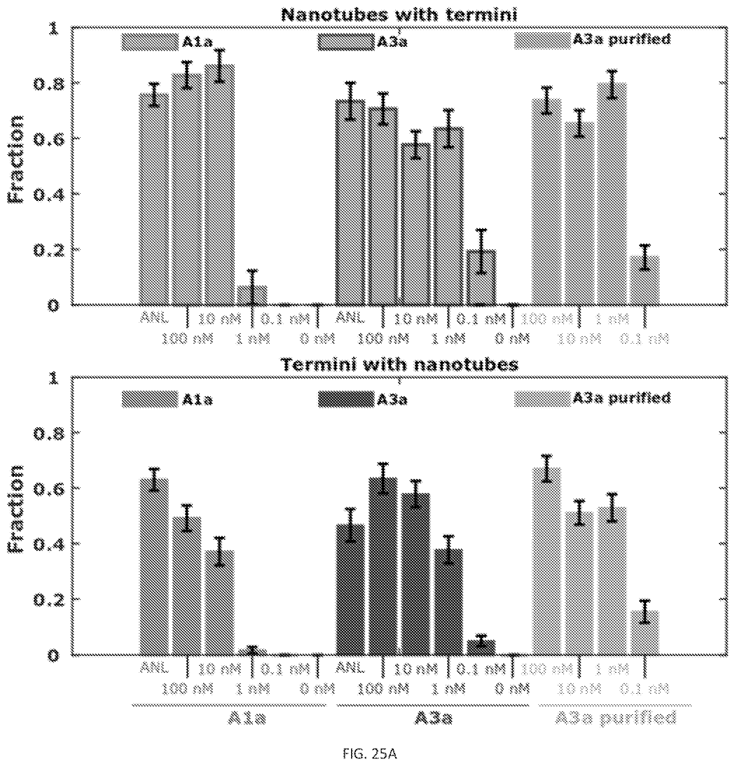

It was thus found that the sequences of the activation strands, which were previously designed to assemble into adapter structures during annealing were predicted to have significant secondary structure, and thus could fold on themselves rather than attach to the ASBS of termini in the isothermal activation experiments at 20° C. (FIG. 24). To investigate whether the presence of this secondary structure was a factor in the high concentration of activation strands needed for terminus activation, another terminus (A3) was designed with activation strands that are predicted to have no significant secondary structure at 20° C. When 1 nM of these activation strands was added to 3 pM of inactive A3 termini and 45 nM monomers, roughly 40% of the A3 termini grew nanotubes, many more than the <2% of A1 termini when 1 nM of A1 activation strands was added to these termini under the same conditions (FIGS. 7A and 25). Yet hardly any A3 activation (<5%) was observed when only 0.1 nM of the activation strands was used. It was theorized this might be because unpurified activation strands were used of which a significant fraction (according to IDT, which synthesized the strands, as many as 30-40% of strands) may have sequence errors. To test whether these sequence errors limited activation, 0.1 nM of PAGE purified A3 activation strands to 3 pM inactive A3 termini and 45 nM monomers were used. After 24 hours of incubation, nearly 20% of A3 termini grew nanotubes (FIGS. 7A and 25). Further, the fraction of A3 termini that grew nanotubes after 0.1 nM of purified strands was added increased to nearly 40% after a longer incubation period (FIG. 25).

In the experiments described above, inactive termini that were activated by adding all six adapter sticky end strands and required two unique sequences for activation (one for the RE adapters and one for the SE adapters) were designed and used. To test whether fewer strands could be used for activation, the nucleation efficiency of termini with 7 different patterns of sticky ends strands on their facets (FIG. 7B) was tested. Termini that were missing just the SE sticky end adapter strands resulted in hardly any nucleation (V5 in FIG. 7B), suggesting that activation could occur through the addition of just the RE sticky end strands which would simplify activation to just a single DNA sequence.

### Example 6

### Molecular Transport Through Self-Assembled DNA Nanofluidic Channels

Nanoscale channels are a fundamental mechanism for directed transport within living systems. Confinement of transport to one dimension makes transport rapid, and gating at channel entrances can make transport selective.

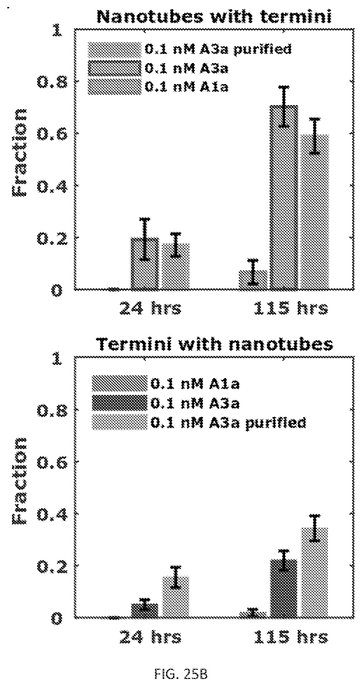

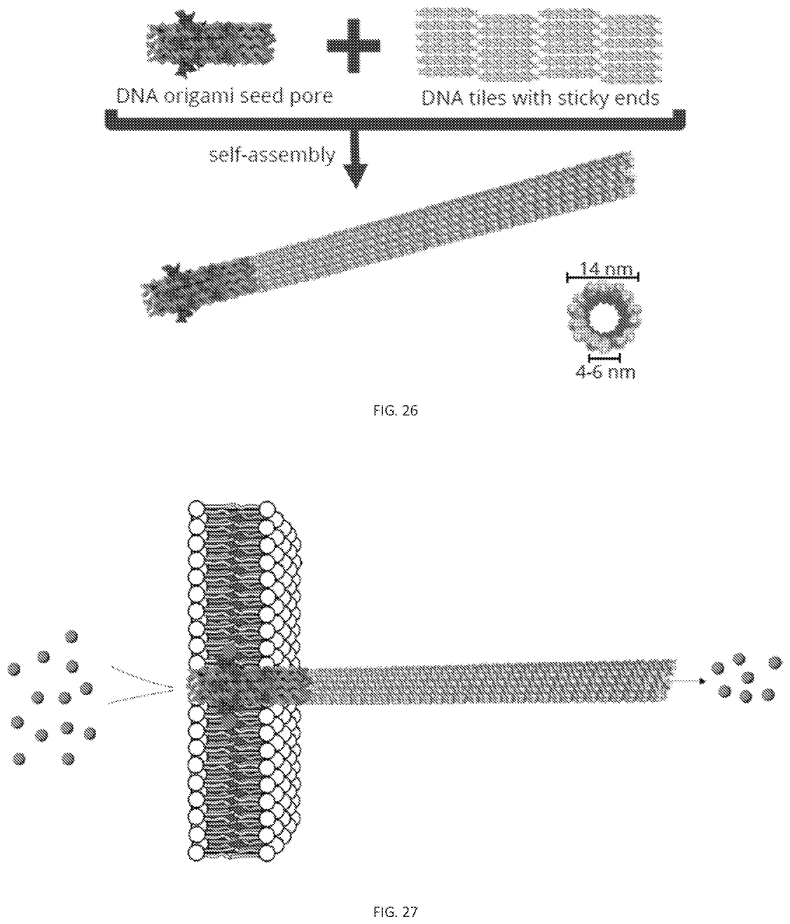

Synthetic nanoscale channels for molecular transport were constructed using DNA nanotubes. The channel has an internal diameter of 4-6 nm and a length that can extend for multiple microns. Lengths were not uniform but can be tuned by different concentrations of DNA tiles during self-assembly. The channels were composed of a DNA origami pore that penetrates and spans a lipid membrane and a DNA nanotube self-assembled from DAE-E double crossover tiles, consisting of five DNA oligomers that grow from the DNA origami pore (FIGS. 26 and 27).

Embedding a DNA assembly into lipid membrane can be achieved by covalent modification of hydrophobic moieties. (See, for example. Langecker, M. et al. Acc. Chem. Res. 2014, incorporated herein by reference in its entirety). Membrane incorporation of the DNA origami pore was facilitated by hydrophobic functionalization of DNA origami pore via 12 cholesterol moieties. The self-assembled channels can self-repair and grow, for example, the endpoints may specifically attach at molecular landmarks.

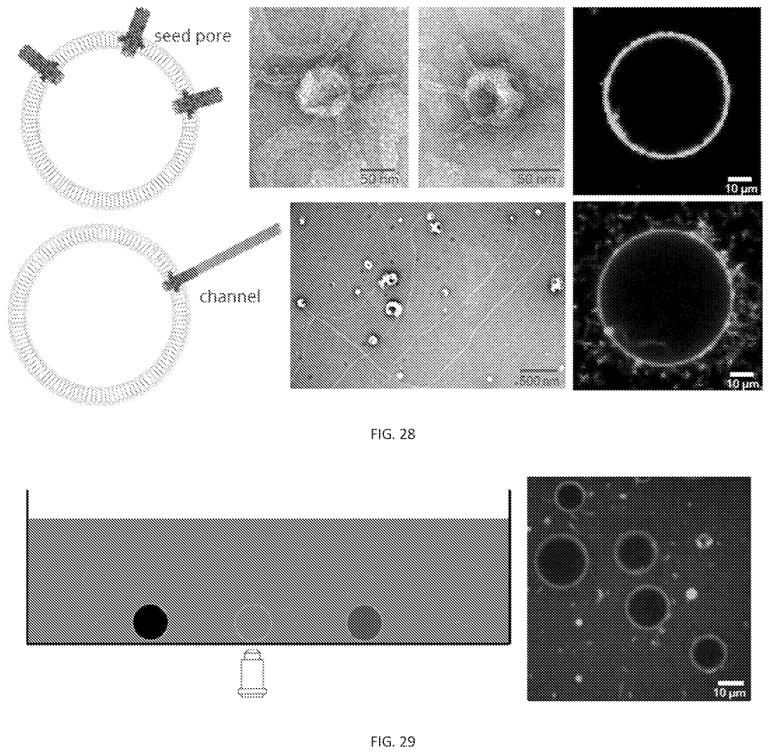

DNA origami pores, with or without attaching to DNA nanotubes, formed channels across lipid membrane after modified with 12 cholesterol moieties. TEM images (FIG. 28, middle) show DNA seed pores and nanotubes inserted into the lipid membrane of small unilamellar vesicles (SUVs).

Giant unilamellar vesicles (GUVs) have low membrane curvatures and are commonly used as an artificial cell model. GUVs are prepared by hydrating hybrid films of lipid and agarose polymer, composed of DPhPC/PE-biotin/PC-BODIPY (44:5:1). DNA origami pores and nanotubes (cyan) could also be anchored in the lipid membranes of GUVs (green) (FIG. 28, right).

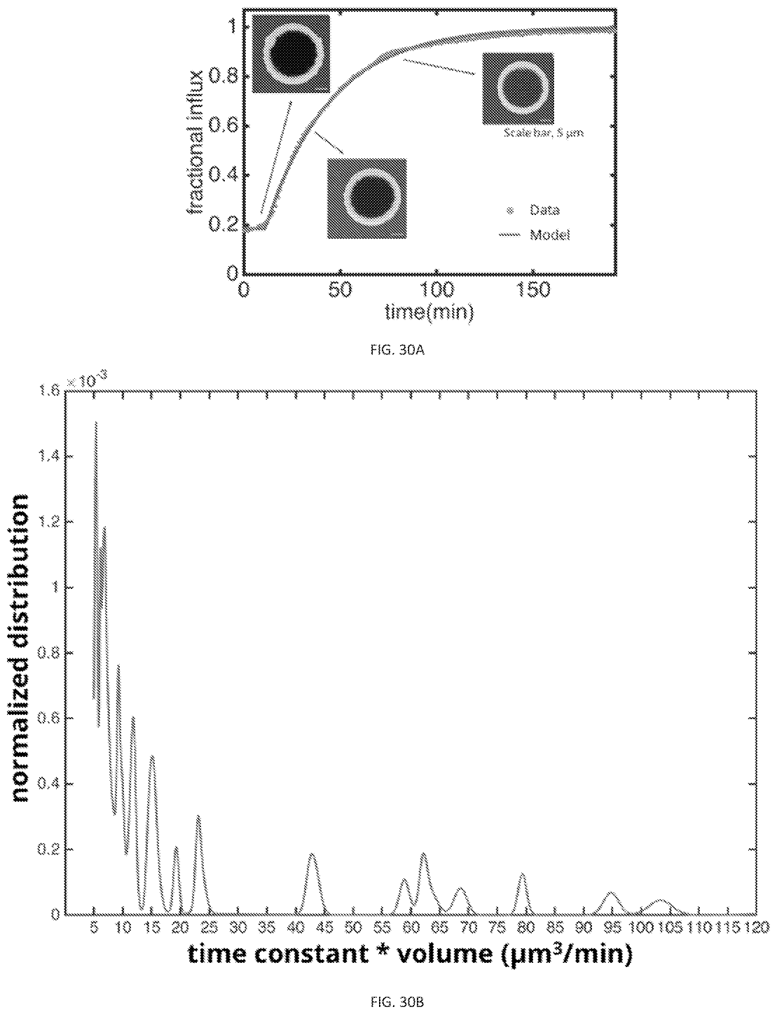

Using a dye influx assay the molecular transport through these DNA channels was characterized as a result of DNA channels spontaneously inserting into GUVs. The fluorescent dyes, initially present only outside of GUVs, were transported into GUVs through the channels penetrating the lipid membranes resulting in increased fluorescent intensity inside GUVs (FIG. 29, left). The influx process, which takes hours to complete, was observed with a fluorescence confocal microscope (FIG. 29, right). For the assay, fluorescent GUVs (green) synthesized with biotinyl lipids were immobilized on a streptavidin-coated glass coverslip, immersed in a solution containing small molecule dyes, rhodamine of 430 Da molecular weight (red), and DNA channels (cyan) in nanomolar concentration.

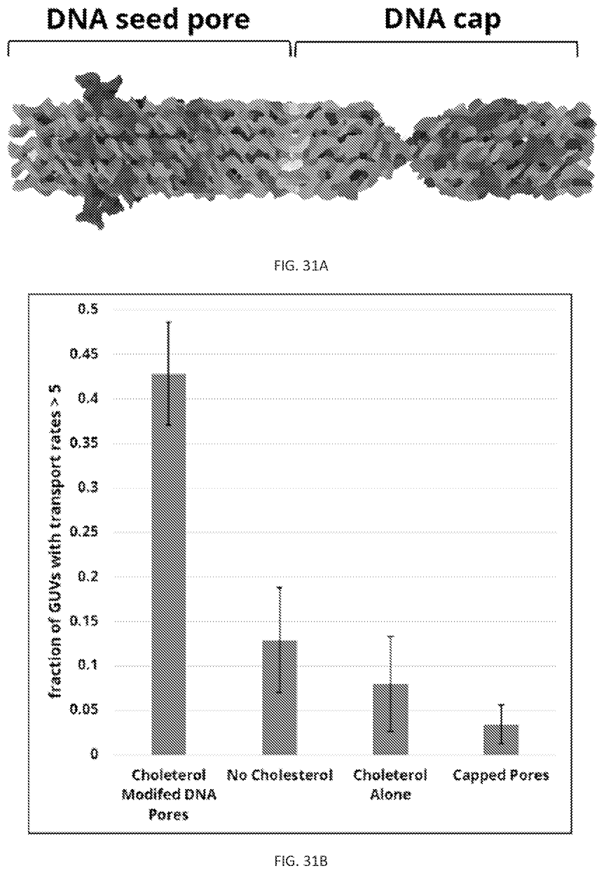

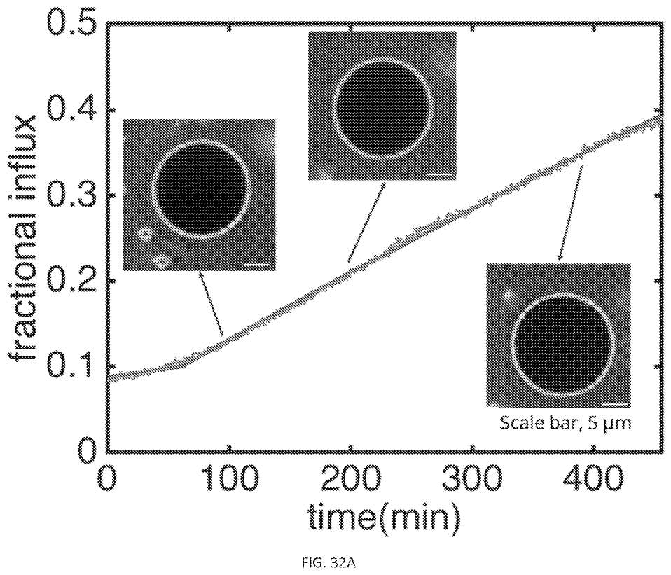

Transport rates of rhodamine molecules through DNA pores were quantified by time-lapse measurement of fluorescent intensity inside GUVs (FIG. 30). The measured rate was compared to a diffusion-dominated molecular transport model, suggesting that the transport was diffusion driven.

The theoretical transport rate per seed pore is:

$$τ ⨯ V = \frac{A ⨯ D}{Δ x} = \frac{d N}{d t} ( \frac{1}{{C}_{out} - {C}_{i n}} ) = \frac{transport rate}{concentration difference} = 14.3 ( \pm 1.5 ) \frac{{μm}^{3}}{min} \\$$

$$f = a ⨯ t + b + [ ( f ( {t}_{0} ) - 1 ) ⨯ {e}^{- τ ⨯ ( t - {t}_{0} )} - ( f {t}_{0} ) - 1 ) ] when t \geq {t}_{0}$$

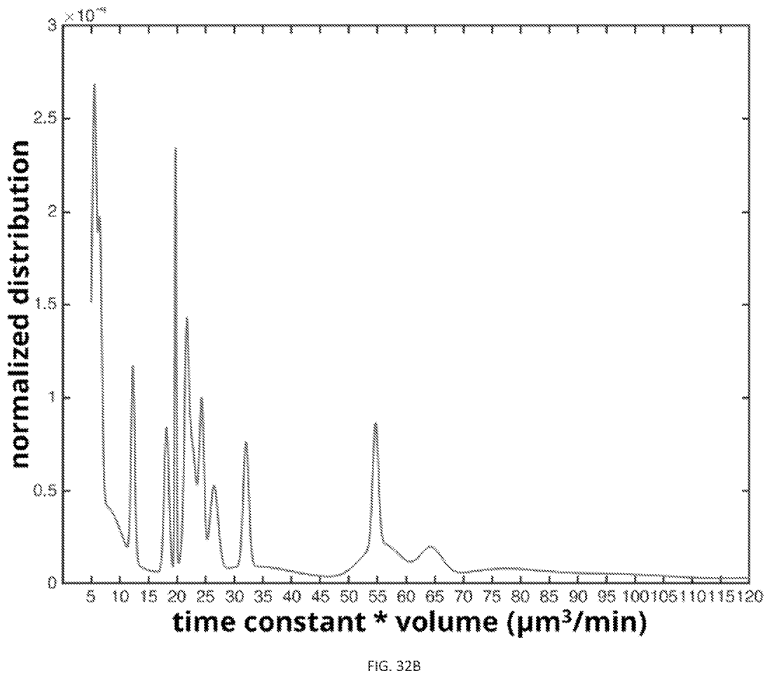

A DNA origami cap was designed (FIG. 31A) that specifically bound to seed pores and stopped transport through the pores. Significantly reduced influx rates resulted when capped seed pores are added to vesicles (FIG. 31B).

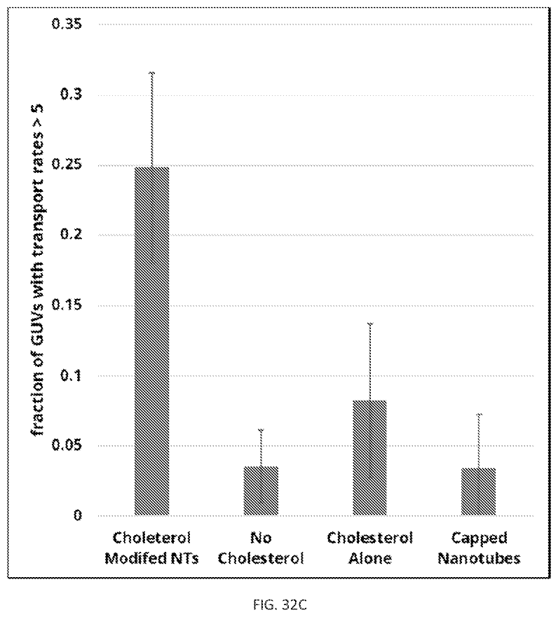

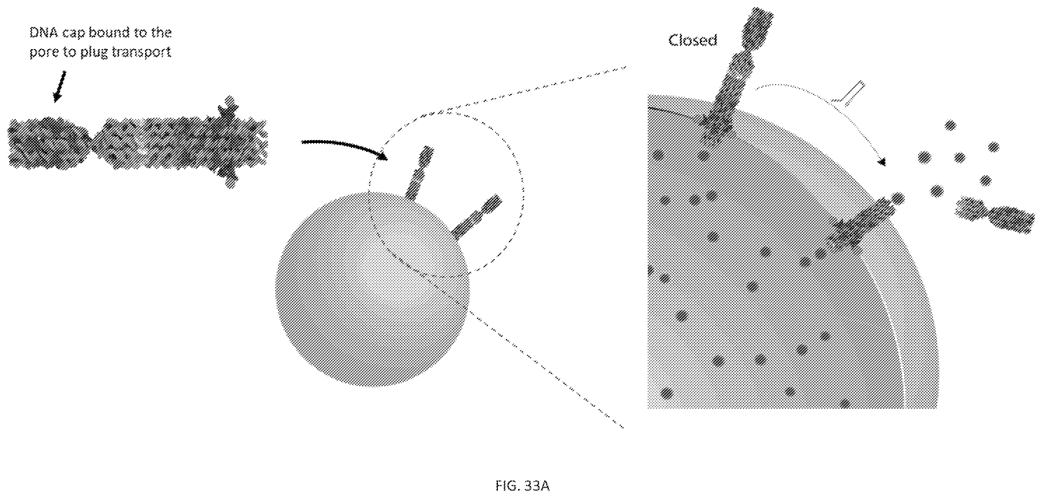

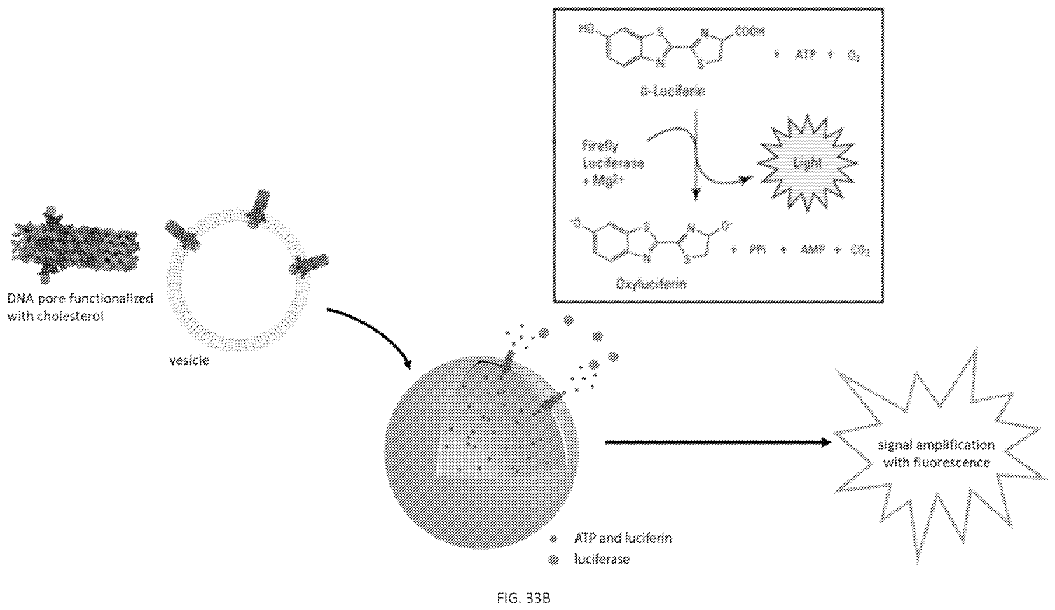

Transport rates through the DNA nanotube channels were lower compared to those through DNA pores, due to longer diffusion distances, and were widely distributed as nanotube lengths vary (FIG. 32).

### Example 7

### Amplification of Chemical Signal Using Responsive DNA Nanofluidic Channels

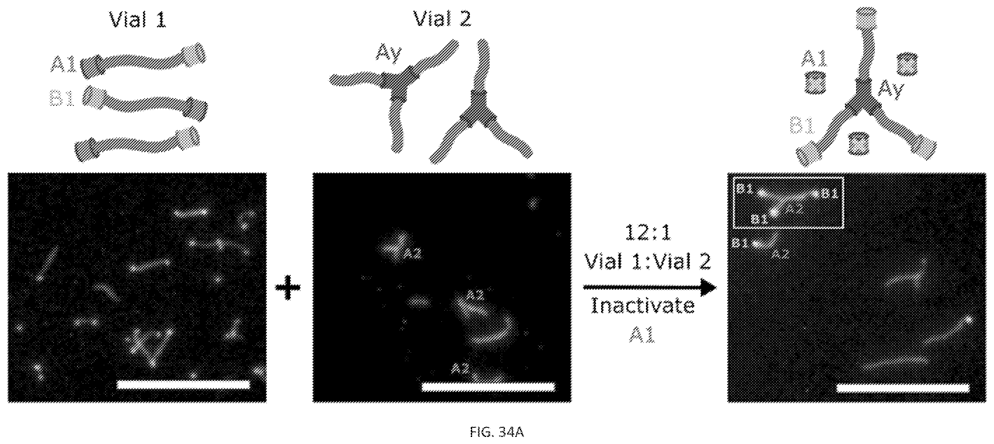

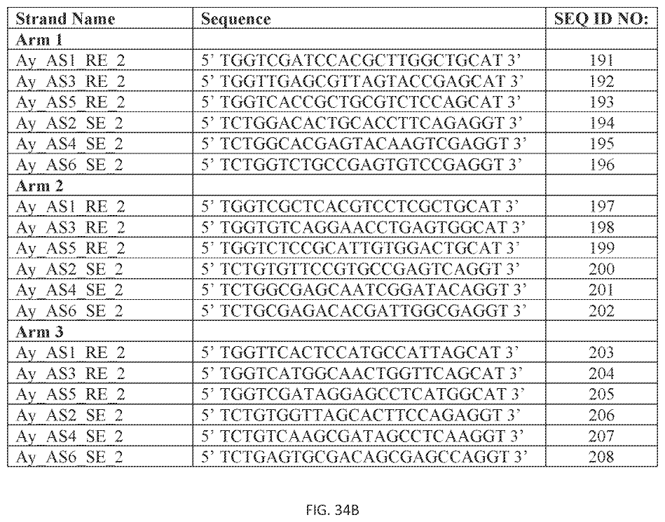

In one exemplary sensor technology, a nanopore embedded in a giant unilamellar vesicle membrane can be opened as a result of a specific chemical molecule (e.g., DNA sequence) facilitating removal of DNA cap on the nanotube thus releasing a large amount of an output molecule (e.g., glucose) or molecules (e.g., ATP and luciferin) into the surrounding solution allowing detection (FIG. 33A). As shown in FIG. 33B, a vesicle may be filled with ATP and luciferin which when released interacts with luciferase in the surrounding solution to result in a measurable fluorescent read-out.

Giant unilamellar vesicles (GUVs) containing glucose (0.1 M) and a fluorescent agent (TAMRA (5 μM) or dextran linked TAMRA (20 μM)) were made using a gel-assisted hydration method (See Hoger, et al., J. Am. Chem. Soc. (2009) Vol. 131. Issue 5, incorporated herein by reference in its entirety). In summary, a thin agarose gel film forms on a glass coverslip and is dried on a heat plate. A dissolution of a specific concentration of a lipid mixture in chloroform solvent is spread onto the agarose film. The solvent in the lipid-agarose hybrid film is evaporated under vacuum overnight. Then, an aqueous solution containing 100 mM glucose is added to the dried film, after which GUVs form over the course of 2-3 days at room temperature. To wash off the glucose outside the GUVs, a magnesium-supplemented PBS buffer (PBSM) solution is added to the GUV solution. A series of centrifugation and supernatant removal lead to GUVs that encapsulate 100 mM glucose suspended in PBSM buffer. DNA nanopores as described herein comprising a cap and an inactivation sequence were included with the GUVs at 37° C. for 3 hours.

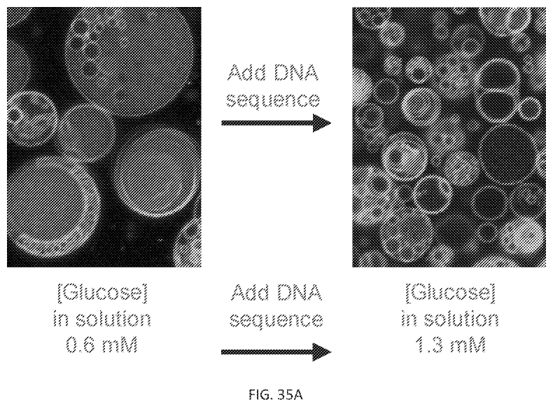

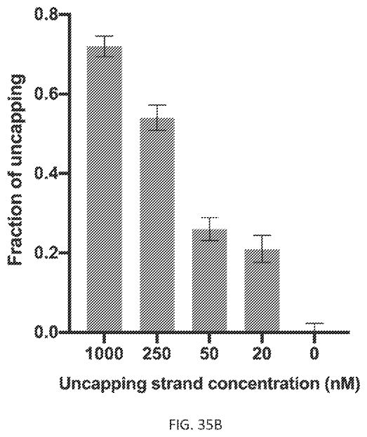

Different 100 mOsm centrifugation buffers each resulted in a small amount of glucose measured in the absence of embedded nanopores using an over-the-counter glucometer. However, in the presence of a DNA strand complementary to the inactivation sequence, the measured glucose increased about 2-4 fold (FIG. 35A). Microscopy experiments also showed that 20 nM of the DNA strand can drive opening of the pores to release glucose (FIG. 35B).

The above experiments were repeated with vesicles (GUVs) containing only glucose. 2 nM DNA pores in vesicles and 1 μM uncapping strand (DNA strand complementary to the inactivation sequence) were used to measure glucose release into the surrounding buffer (PBSM) at various timepoints with an over-the-counter glucometer. The vesicles were then sonicated after the final timepoint to measure total glucose and compute fractional glucose release for each timepoint (Tables 1 and 2).

Fractional glucose release can be influenced by a number of factors: varying total number vesicles; varying DNA pore insertion efficiency, such that some vesicles do not have an inserted pore; and larger number of total vesicles leading to low fractional release, even with high levels of signal amplification. However, baseline glucose release was found to be proportional to the vesicle amount. Thus, glucose concentration release was calibrated by calculating the glucose above baseline as shown in the following equation:

Calibrated glucose concentration=measured glucose concentration−total glucose concentration×baseline leak fraction

TABLE 1

| | | | | | | After |
|---|---|---|---|---|---|---|
| | 0 h | 1 h | 2 h | 3 h | 8 h | Sonication |
| Glucose | <0.4 | 1.7 | 2.4 | 2.7 | 4.5 | 9.8 |
| Concentration (mM) | | | | | | |
| Fractional | <0.04 | 0.18 | 0.25 | 0.28 | 0.45 | |
| Release | | | | | | |
| Calibrated | 0 | 0.7 | 1.1 | 1.3 | 2.5 | |
| Glucose (mM) | | | | | | |

TABLE 2

| | | | | | | After |
|---|---|---|---|---|---|---|
| | 0 h | 1 h | 2 h | 3 h | 8 h | Sonication |
| Glucose | <0.4 | 1.2 | 1.4 | 1.7 | 2.7 | 3.5 |
| Concentration (mM) | | | | | | |
| Fractional | <0.11 | 0.35 | 0.39 | 0.49 | 0.76 | |
| Release | | | | | | |
| Calibrated | 0 | 0.8 | 0.9 | 1.2 | 2.0 | |
| Glucose (mM) | | | | | | |

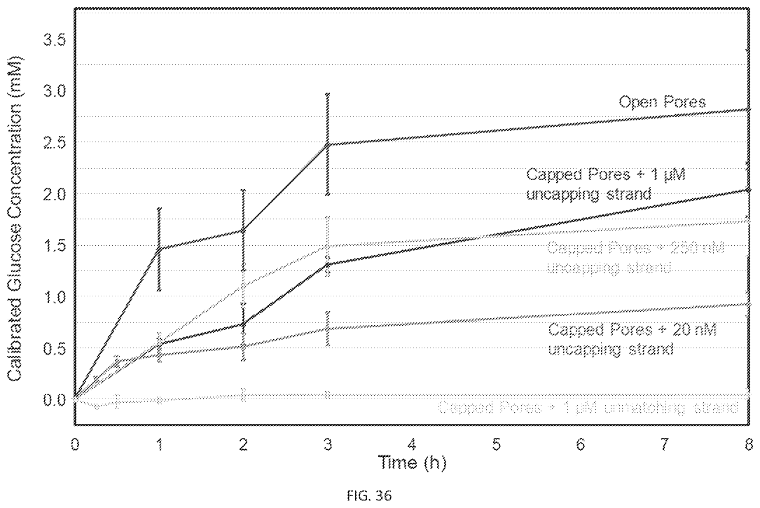

Vesicles comprising capped pores were incubated with varying concentrations of uncapping strand and a random-sequence control strand. As shown in FIG. 36, the uncapping strand triggered glucose release but not the random-sequence control strand. 20 nM uncapping strand DNA triggered a release of 307 μM glucose into the solution in 30 minutes; a 18,500× amplification of the molecular (uncapping strand) signal.

### Example 8

### Terminus Inactivation for Step-Wise Assembly of Hierarchical Nanostructures

It was previously demonstrated that multi-armed DNA nanotube structures with specific geometries may be nucleated from DNA origami seeds with multiple adapter interfaces presented at specific angles. These structures offer the possibility of constructing branching dendritic nanotube structures akin to cytoskeleton growth. However, a key feature of cytoskeletal networks is the ability to dynamically rearrange and either expand or contract depending on cellular conditions. Terminus activation/inactivation provides an ideal mechanism for mimicking this kind of behavior.

A simplified process of this activation and inactivation was used herein: the end-to-end joining of a population of three-armed “Y” nanotube structures nucleated from Y-shaped DNA origami termini presenting the A interface with a population of one-dimensional nanotube structures. The one-dimensional nanotube structures were first grown from A1 seeds over 20 hr and then terminated via the addition of B1 termini (FIG. 34, vial 1). The resulting structures were then mixed in 12-fold excess with pre-grown Y nanotube structures (FIG. 34, vial 2). The A1-B1 doubly terminated nanotubes were protected from joining to the Y nanotube structures. To deprotect the A1-B1 terminated structures, the A1 termini were inactivated which allowed joining of the nanotubes to the Y structures (FIG. 34, right side). The procedure outlined above may be used to step-wise assemble branching DNA nanotube architectures.

The Y-shaped DNA origami termini used here are identical to those presented previously (T. D. Jorgenson, et. al., ACS Nano, 2017, 11, 1927-1936, incorporated herein by reference in its entirety) except for the activation strands, which present 4-base sticky ends. The staples, labelling strands, and labelling attachment strands all match those used previously. The activation used herein are provided in FIG. 34B.

### REFERENCES

(1) Fletcher. D. A.; Mullins, R. D. Cell Mechanics and the Cytoskeleton. Nature 2010, 463 (7280), 485-492. https://doi.org/10.1038/nature08908.
(2) Petry. S.; Vale. R. D. Microtubule Nucleation at the Centrosome and Beyond. Nat. Cell Biol. 2015, 17, 1089-1093. https://doi.org/10.1038/ncb3220.
(3) Blanchoin. L.; Boujemaa-Paterski. R.; Sykes. C.; Plastino, J. Actin Dynamics. Architecture. and Mechanics in Cell Motility. Physiol. Rev. 2014, 94 (1), 235-263. https://doi.org/10.1152/physrev.00018.2013.
(4) Basnet. N.; Nedozralova. H.; Crevenna, A. H.; Bodakuntla, S.; Schlichthaerle, T.; Taschner, M.; Cardone, G.; Janke. C.; Jungmann. R.; Magiera. M. M.; Biertümpfel, C.; Mizuno. N. Direct induction of Microtubule Branching by Microtubule Nucleation Factor SSNA1. Nat. Cell Biol. 2018, 20 (10), 1172-1180. https://doi.org/10.1038/s41556-018-0199-8.
(5) Kapitein. L. C.; Hoogenraad, C. C. Building the Neuronal Microtubule Cytoskeleton. Neuron 2015, 87 (3), 492-506. https://doi.org/10.1016/j.neuron.2015.05.046.
(6) Bailey, C. H.; Kandel. E. R. Chapter 10 Synaptic Remodeling, Synaptic Growth and the Storage of Long-Term Memory in Aplysia. In Progress in Brain Research; Sossin, W. S., Lacaille. J.-C., Castellucci, V. F., Belleville, S., Eds.; Elsevier, 2008; Vol. 169, pp 179-198. https://doi.org/10.1016/S0079-6123(07)00010-6.
(7) Maune. H. T.; Han. S.; Barish, R. D.; Bockrath, M.; Goddard. I. Iiw. A.; RothemundPaul. W. K.; Winfree. E. Self-Assembly of Carbon Nanotubes into Two-Dimensional Geometries Using DNA Origami Templates. Nat. Nanotechnol. 2010, S (1), 61-66. https://doi.org/10.1038/nnano.2009.311.
(8) Sanchez-Valencia, J. R.; Dienel. T.; Gröning, O.; Shorubalko, I.; Mueller, A.; Jansen, M.; Amsharov. K.; Ruffieux, P.; Fasel, R. Controlled Synthesis of Single-Chirality Carbon Nanotubes. Nature 2014, 512, 61. https://doi.org/10.1038/nature13607.
(9) Serra. M.; Arenal, R.; Tenne, R. An Overview of the Recent Advances in Inorganic Nanotubes. Nanoscale 2019, 11 (17), 8073-8090. https://doi.org/10.1039/C9NR01880H.
(10) Shen. H.; Fallas, J. A.; Lynch, E.; Sheffler. W.; Parry, B.; Jannetty, N.; Decarreau. J.; Wagenbach, M.; Vicente, J. J.; Chen, J.; Wang. L.; Dowling, Q.; Oberdorfer, G.; Stewart, L.; Wordeman. L.; De Yoreo. J.; Jacobs-Wagner. C.; Kollman. J.; Baker, D. De Novo Design of Self-Assembling Helical Protein Filaments. Science 2018, 362 (6415), 705-709. https://doi.org/10.1126/science.aau3775.
(11) Nishinaka, T.; Takano. A.; Doi. Y.; Hashimoto. M.; Nakamura, A.; Matsushita, Y.; Kumaki. J.; Yashima, E. Conductive Metal Nanowires Templated by the Nucleoprotein Filaments, Complex of DNA and RecA Protein. J. Am. Chem. Soc. 2005, 127(22), 8120-8125. https://doi.org/10.1021/ja050487h.
(12) Brodin, J. D.; Smith, S. J.; Carr, J. R.; Tezcan, F. A. Designed. Helical Protein Nanotubes with Variable Diameters from a Single Building Block. J. Am. Chem. Soc. 2015, 137 (33), 10468-10471. https://doi.org/10.1021/jacs.5b05755.
(13) Rothemund, P. W. K.; Ekani-Nkodo, A.; Papadakis, N.; Kumar, A.; Fygenson. D. K.; Winfree, E. Design and Characterization of Programmable DNA Nanotubes. J. Am. Chem. Soc. 2004, 126 (50), 16344-16352. https://doi.org/10.1021/ja0443191.
(14) Mohammed, A. M.; Schulman, R. Directing Self-Assembly of DNA Nanotubes Using Programmable Seeds. Nano Lett. 2013, 13 (9), 4006-4013. https://doi.org/10.1021/nl400881w.
(15) Liu, D.; Park, S. H.; Reif. J. H.; LaBean, T. H. DNA Nanotubes Self-Assembled from Triple-Crossover Tiles as Templates for Conductive Nanowires. Proc. Nat. Acad. Sci. U.S.A 2004, 101 (3), 717-722. https://doi.org/10.1073/pnas.0305860101.
(16) Han, D.; Pal. S.; Nangreave, J.; Deng, Z.; Liu. Y.; Yan. H. DNA Origami with Complex Curvatures in Three-Dimensional Space. Science 2011, 332 (6027), 342-346. https://doi.org/10.1126/science.1202998.
(17) Rothemund, P. W. K. Folding DNA to Create Nanoscale Shapes and Patterns. Nature 2006, 440 (7082), 297-302. https://doi.org/10.1038/nature04586.
(18) Wei, B.; Dai, M.; Yin, P. Complex Shapes Self-Assembled from Single-Stranded DNA Tiles. Nature 2012, 485 (7400), 623-626. https://doi.org/10.1038/nature11075.
(19) Ke, Y.; Ong. L. L.; Shih. W. M.; Yin. P. Three-Dimensional Structures Self-Assembled from DNA Bricks. Science 2012, 338 (6111), 1177. https://doi.org/10.1126/science.1227268.
(20) Lo, P. K.; Karam. P.; Aldaye, F. A.; McLaughlin, C. K.; Hamblin. G. D.; Cosa, G.; Sleiman, H. F. Loading and Selective Release of Cargo in DNA Nanotubes with Longitudinal Variation. Nat. Chem. 2010, 2 (4), 319-328. https://doi.org/10.1038/nchem.575.
(21) Wilner, O. I.; Orbach, R.; Henning, A.; Teller. C.; Yehezkeli, O.; Mertig. M.; Harries. D.; Willner, I. Self-Assembly of DNA Nanotubes with Controllable Diameters. Nat. Commun. 2011, 2, 540. https://doi.org/10.1038/ncomms1535.
(22) Peng Yin; Hariadi, R. F.; Sahu, S.; Choi, H. M. T.; Sung Ha Park; Labean. T. H.; Reif. J. H. Programming DNA Tube Circumferences. Science 2008, 321 (5890), 824-826.https://doi.org/10.1126/science.1157312.
(23) Jorgenson, T. D.; Mohammed, A. M.; Agrawal. D. K.; Schulman. R. Self-Assembly of Hierarchical DNA Nanotube Architectures with Well-Defined Geometries. ACS Nano 2017, 11 (2), 1927-1936. https://doi.org/10.1021/acsnano.6b08008.
(24) Agrawal, D. K.; Jiang. R.; Reinhart, S.; Mohammed. A. M.; Jorgenson, T. D.; Schulman, R. Terminating DNA Tile Assembly with Nanostructured Caps. ACS Nano 2017, 11 (10), 9770-9779. https://doi.org/10.1021/acsnano.7b02256.
(25) Mohammed, A. M.; Velazquez, L.; Chisenhall, A.; Schiffels, D.; Fygenson, D. K.; Schulman, R. Self-Assembly of Precisely Defined DNA Nanotube Superstructures Using DNA Origami Seeds. Nanoscale 2017, 9 (2), 522-526. https://doi.org/10.1039/C6NR06983E.
(26) Mohammed, A. M.; § ulc, P.; Zenk. J.; Schulman, R. Self-Assembling DNA Nanotubes to Connect Molecular Landmarks. Nat. Nanotechnol. 2017, 12 (4), 312-316. https://doi.org/10.1038/nnano.2016.277.
(27) Zhang, D. Y.; Seelig, G. Dynamic DNA Nanotechnology Using Strand-Displacement Reactions. Nat. Chem. 2011, 3 (2), 103-113. https://doi.org/10.1038/nchem.957.
(28) Wang, M.; Dong, J.; Zhou. C.; Xie. H.; Ni, W.; Wang, S.; Jin, H.; Wang. Q. Reconfigurable Plasmonic Diastereomers Assembled by DNA Origami. ACS Nano 2019, 13 (12), 13702-13708. https://doi.org/10.1021/acsnano.9b06734.
(29) Petersen, P.; Tikhomirov, G.; Qian, L. Information-Based Autonomous Reconfiguration in Systems of Interacting DNA Nanostructures. Nat. Commun. 2018, 9 (1), 5362. https://doi.org/10.1038/s41467-018-07805-7.
(30) Pearson. A. C.; Liu, J.; Pound, E.; Uprety. B.; Woolley, A. T.; Davis, R. C.; Harb, J. N. DNA Origami Metallized Site Specifically to Form Electrically Conductive Nanowires. J. Phys. Chem. B 2012, 116 (35), 10551-10560. https://doi.org/10.1021/jp302316p.
(31) Kuzyk, A.; Schreiber. R.; Fan, Z.; Pardatscher, G.; Roller, E.-M.; Hogele, A.; Simmel, F. C.; Govorov, A. O.; Liedl, T. DNA-Based Self-Assembly of Chiral Plasmonic Nanostructures with Tailored Optical Response. Nature 2012, 483 (7389), 311-314. https://doi.org/10.1038/nature10889.
(32) Bui, H.; Onodera, C.; Kidwell, C.; Tan, Y.; Graugnard, E.; Kuang. W.; Lee, J.; Knowlton. W. B.; Yurke, B.; Hughes, W. L. Programmable Periodicity of Quantum Dot Arrays with DNA Origami Nanotubes. Nano Lett. 2010, 10 (9), 3367-3372. https://doi.org/10.1021/nI101079u.
(33) Chandrasekaran. A. R. Programmable DNA Scaffolds for Spatially-Ordered Protein Assembly. Nanoscale 2016, 8 (8), 4436-4446. https://doi.org/10.1039/C5NR08685J.
(34) Sharma, J.; Chhabra, R.; Cheng, A.; Brownell, J.; Liu, Y.; Yan. H. Control of Self-Assembly of DNA Tubules Through Integration of Gold Nanoparticles. Science 2009, 323 (5910), 112-116. https://doi.org/10.1126/science.1165831.
(35) Fu, T. J.; Seeman, N. C. DNA Double-Crossover Molecules. Biochemistry 1993, 32 (13), 3211-3220. https://doi.org/10.1021/bi00064a003.
(36) Zhang. D. Y.; Winfree, E. Control of DNA Strand Displacement Kinetics Using Toehold Exchange. J. Am. Chem. Soc. 2009, 131 (47), 17303-17314. https://doi.org/10.1021/ja906987s.
(37) Ekani-Nkodo. A.; Kumar. A.; Fygenson, D. K. Joining and Scission in the Self-Assembly of Nanotubes from DNA Tiles. Phys. Rev. Lett. 2004, 93 (26), 268301. https://doi.org/10.1103/PhysRevLett.93.268301.
(38) Zhang. D. Y.; Hariadi. R. F.; Choi. H. M. T.; Winfree, E. Integrating DNA Strand-Displacement Circuitry with DNA Tile Self-Assembly. Nat. Commun. 2013, 4, 1965. https://doi.org/10.1038/ncomms2965.
(39) Ranallo. S.; Sorrentino, D.; Ricci. F. Orthogonal Regulation of DNA Nanostructure Self-Assembly and Disassembly Using Antibodies. Nat. Commun. 2019, 10 (1), 5509. https://doi.org/10.1038/s41467-019-13104-6.
(40) Minev, D.; Wintersinger, C. M.; Ershova. A.; Shih. W. M. Robust Nucleation Control via Crisscross Polymerization of DNA Slats. bioRxiv 2019. https://doi.org/10.1101/2019.12.11.873349.
(41) Gates, E. P.; Jensen, J. K.; Harb. J. N.; Woolley. A. T. Optimizing Gold Nanoparticle Seeding Density on DNA Origami. RSC Adv. 2015, 5 (11), 8134-8141. https://doi.org/10.1039/C4RA15451G.
(42) Green. L. N.; Subramanian, H. K. K.; Mardanlou, V.; Kim, J.; Hariadi, R. F.; Franco, E. Autonomous Dynamic Control of DNA Nanostructure Self-Assembly. Nat. Chem. 2019, 11 (6), 510-520. https://doi.org/10.1038/s41557-019-0251-8.
(43) Fern, J.; Scalise. D.; Cangialosi. A.; Howie, D.; Potters. L.; Schulman. R. DNA Strand-Displacement Timer Circuits. ACS Synth. Biol. 2017, 6 (2), 190-193. https://doi.org/10.1021/acssynbio.6b00170.
(44) Scalise, D.; Rubanov, M.; Miller. K.; Potters. L.; Noble. M.; Schulman. R. Programming the Sequential Release of DNA. ACS Synth. Biol. 2020, 9 (4), 749-755. https://doi.org/10.1021/acssynbio.9b00398.
(45) Cherry, K. M.; Qian. L. Scaling up Molecular Pattern Recognition with DNA-Based Winner-Take-All Neural Networks. Nature 2018, 559 (7714), 370-376. https://doi.org/10.1038/s41586-018-0289-6.
(46) Yu, T.; Wei. Q. Plasmonic Molecular Assays: Recent Advances and Applications for Mobile Health. Nano Res. 2018, 11 (10), 5439-5473. https://doi.org/10.1007/s12274-018-2094-9.
(47) Tan. S. J.; Campolongo, M. J.; Dan. L.; Wenlong, C. Building Plasmonic Nanostructures with DNA. Nat. Nanotechnol. 2011, 6 (5), 268-276. https://doi.org/10.1038/nnano.2011.49.
(48) Taton, T. A.; Mirkin. C. A.; Letsingcr, R. L. Scanometric DNA Array Detection with Nanoparticle Probes. Science 2000, 289 (5485), 1757. https://doi.org/10.1126/science.289.5485.1757.
(49) Fern, J.; Schulman. R. Modular DNA Strand-Displacement Controllers for Directing Material Expansion. Nat. Commun. 2018, 9 (1), 3766. https://doi.org/10.1038/s41467-018-06218-w.
(50) Seelig. G.; Soloveichik, D.; Zhang, D. Y.; Winfree, E. Enzyme-Free Nucleic Acid Logic Circuits. Science 2006, 314 (5805), 1585-1588. https://doi.org/10.1126/science.1132493.
(51) Sellner, S.; Kocabey. S.; Nekolla, K.; Krombach, F.; Liedl. T.; Rehberg. M. DNA Nanotubes as Intracellular Delivery Vehicles in Vivo. Biomaterials 2015, 53, 453-463. https://doi.org/10.1016/j.biomaterials.2015.02.099.
(52) J. N. Zadeh. C. D. Steenberg. J. S. Bois, B. R. Wolfe. M. B. Pierce, A. R. Khan. R. M. Dirks and N. A. Pierce, J. Comput. Chem., 2011, 32, 170-173.
(53) J. Canny. IEEE Transactions on Pattern Analysis and Machine Intelligence, 1986. PAMI-8, 679-698.
(54) Göpfrich, K. et al. ACS nano. 2016.
(55) Langecker. M. et al. Science. 2012.
(56) Bruns, J. R. Nano Lett. 2013.
(57) Langecker. M. et al. Acc. Chem. Res. 2014.
(58) Horger. K. et al. J. Am. Chem. Soc. 2009.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A system comprising:
a nucleic acid nanotube, nucleic acid nanotube seed, a plurality nucleic acid nanotube monomers, or a combination thereof;
a nucleic acid nanotube terminus; and
an activation strand comprising a single-stranded nucleic acid comprising a first sequence complementary to a portion of the nucleic acid nanotube, nucleic acid nanotube seed, nucleic acid nanotube monomers and a second sequence complementary to a portion of the nucleic acid nanotube terminus;
wherein the nucleic acid nanotube terminus lacks nucleic acid strands configured to interact with the nucleic acid nanotube, the nucleic acid nanotube seed, or the plurality of nucleic acid nanotube monomers.

2. The system of claim 1, wherein the nanotube terminus comprises activation strand binding sites comprising nucleic acid sequences complementary to the second sequence of the activation strand.

3. The system of claim 1 or claim 2, wherein the nucleic acid nanotube terminus comprises a capped end.

4. The system of any of claims 1-3, wherein the single-stranded nucleic acid of the activation strand comprises 14-50 bases.

5. The system of any of claims 1-4, wherein the activation strand lacks predicted secondary structure.

6. The system of any of claims 1-5, wherein the system comprises less than 1 nM of the activation strand.

7. The system of any of claims 1-6, wherein the nucleic acid nanotube or nanotube seed and nucleic acid nanotube terminus are linked as one continuous structure by hybridization with the activation strand.

8. The system of claim 7, wherein the activation strand further comprises a toehold sequence not complementary to a portion of the nucleic acid nanotube, the nucleic acid nanotube seed, the plurality of nucleic acid nanotube monomers or the nucleic acid nanotube terminus.

9. The system of any of claims 1-8, wherein the toehold sequence comprises at least a 7-base single-stranded sequence at the 5′ end of the activation strand.

10. The system of any of claims 1-9, wherein the system further comprises an inactivation strand comprising a single-stranded nucleic acid having a sequence complementary to a portion of the activation strand.

11. The system of claim 9 or claim 10, wherein the inactivation strand is complementary to the toehold sequence.

12. The system of any of claims 1-11, wherein the single-stranded nucleic acid of the inactivation strand is at least about 5 bases longer than the activation strand.

13. The system of any of claims 1-12, wherein the nanotube or nanotube seed comprises cholesterol moieties on an outer surface.

14. The system of claim 1-13, wherein the nanotube or nanotube seed is embedded in and spans a lipid membrane.

15. The system of claim 14, wherein the lipid membrane is the lipid membrane of a vesicle.

16. The system of claim 14, wherein the vesicle is a small unilamellar vesicle (SUV) or a giant unilamellar vesicle (GUV).

17. The system of claim 15 or 16, wherein the vesicle encapsulates an analyte.

18. The system of claim 14, wherein the lipid membrane is a cell membrane.

19. The system of any of claims 1-18, wherein the activation and/or inactivation strands are provided as a double-stranded nucleic acid complex comprising the activation or inactivation strand hybridized to one or more partially complementary single-stranded oligonucleotides.

20. The system of claim 19, further comprising biological sample.

21. The system of claim 20, wherein the biological sample comprises a biomarker configured to interact with the double-stranded nucleic acid complex facilitating release of the activation or inactivation strand.

22. The system of claim 21, wherein the biomarker comprises a nucleic acid strand comprising a sequence complementary to the one or more partially complementary single-stranded oligonucleotides.

23. The system of claim 21 or 22, wherein the biomarker comprises a nucleic acid from a pathogenic agent.

24. A method for detecting a biomarker of interest comprising:
providing a composition comprising a nucleic acid nanotube, nucleic acid nanotube seed, nucleic acid nanotube monomers, or a combination there of, and a nucleic acid nanotube terminus, wherein the nucleic acid nanotube terminus lacks nucleic acid strands configured to interact with a nanotubes, a nanotube seed, or nanotube monomers;
incubating the composition with a biological sample comprising a biomarker of interest; and
measuring nanotube growth.

25. The method of claim 24, wherein the composition further comprises a double-stranded nucleic acid complex comprising an activation strand hybridized to one or more partially complementary single-stranded oligonucleotides,
wherein the activation strand is a single-stranded nucleic acid comprising a first sequence complementary to a portion of the nucleic acid nanotube, nucleic acid nanotube seed, nucleic acid nanotube monomers, and a second sequence complementary to a portion of the nucleic acid nanotube terminus.

26. The method of claim 25, wherein the biomarker of interest interacts with the double-stranded nucleic acid complex to release the activation strand.

27. The method of any of claims 24-26, wherein the biomarker of interest is a nucleic acid biomarker.

28. The method of any of claims 24-27, wherein the biomarker of interest is a nucleic acid from a pathogenic agent.

29. The method of claim 27 or 28, wherein, the nucleic acid biomarker comprises 10-50 bases.

30. The method of any of claims 27-29, wherein the biological sample comprises less than InM of the nucleic acid biomarker.

31. The method of any of claims 24-30, wherein the composition comprises 1-100 pM nucleic acid nanotube terminus.

32. A method detecting a biomarker of interest comprising:
providing a composition comprising:
a nanotube or nanotube seed comprising a capped nanotube terminus and an activation strand comprising a toehold sequence; and
a vesicle comprising an encapsulated analyte,
wherein the nanotube or nanotube seed is embedded in the vesicle lipid membrane;
incubating the composition with a biological sample comprising the biomarker of interest; and
measuring release of the analyte from the vesicle.

33. The method of claim 32, the nanotube or nanotube seed further comprises cholesterol moieties on an outer surface.

34. The method of claim 32 or 33, wherein the toehold sequence comprises at least a 7-base single-stranded sequence at the 5′ end of the activation strand.

35. The method of any of claims 32-34, wherein the composition further comprises a double-stranded nucleic acid complex comprising an inactivation strand hybridized to one or more partially complementary single-stranded oligonucleotides,
wherein the inactivation strand is complementary to the toehold sequence.

36. The method of claim 35, wherein the biomarker of interest interacts with the double-stranded nucleic acid complex to release the inactivation strand.

37. The method of any of claims 32-36, wherein the biomarker of interest is a nucleic acid from a pathogenic agent.

38. The method of any of claims 32-37, wherein the biomarker of interest is a single-stranded nucleic acid.

39. The method of any of claim 38, wherein the sample comprises less than 100 nM of the single-stranded nucleic acid biomarker.

40. The method of claims 38 or 39, wherein the sample comprises less than 1 nM of the single-stranded nucleic acid biomarker.

41. The method of any of claims 38-40, wherein the single-stranded nucleic acid biomarker comprises 10-50 bases.

42. The method of any of claims 38-41, wherein the single-stranded nucleic acid biomarker is complementary to the toehold sequence.

43. The method of any of claims 32-42, wherein the vesicle comprises a small unilamellar vesicle (SUV) or a giant unilamellar vesicle (GUV).

44. The method of any of claims 32-43, wherein the analyte comprises a dye, a small molecule detectable agent, a detectable moiety, or a combination thereof.

45. The method of claim 44, wherein the dye comprises a fluorescent dye, a UV dye, or a visible dye.

46. The method of claim 44, wherein the analyte comprises glucose.

47. The method of any of claim 32-46, wherein the composition further comprises a moiety in a solution surrounding the vesicles configured to interact with the analyte and produce a measurable signal.

48. A device comprising the system of any of claims 1-23.
